(19) United States
(12) Patent Application Publication
(10) Pub. No.: US 2022/0088222 A1
(43) Pub. Date: Mar. 24, 2022

(54) COMPOSITIONS AND METHODS FOR THE TREATMENT OF DEGENERATIVE OCULAR DISEASES

(71) Applicants: President and Fellows of Harvard College, Cambridge, MA (US); Massachusetts Ear and Eye Infirmary, Boston, MA (US)

(72) Inventors: Constance L. Cepko, Newton, MA (US); David Mingdar Wu, Cambridge, MA (US)

(21) Appl. No.: 17/414,425

(22) PCT Filed: Dec. 18, 2019
(86) PCT No.: PCT/US2019/067131

§ 371 Date: Jun. 16, 2021

Related U.S. Application Data
(60) Provisional application No. 62/782,584, filed on Dec. 20, 2018.

(51) Int. Cl. A61K 48/00; C12N 15/86; A61P 27/12

(52) U.S. Cl. CPC A61K 48/00; C12N 15/86; C12N 2750/14133; C12N 2750/14143; C12N 2750/14122; A61P 27/12

(57) ABSTRACT

The present invention provides compositions, e.g., pharmaceutical compositions, which include a recombinant adeno-associated viral (AAV) expression construct, AAV vectors, AAV particles, and methods of treating a subject having a degenerative ocular disorder, e.g., retinitis pigmentosa.

### RELATED APPLICATIONS

This application claims the benefit of priority to U.S. Provisional Patent Application No. 62/782,584, filed on Dec. 20, 2018, the entire contents of which are incorporated herein by reference.

### GOVERNMENT FUNDING

This invention was made with Government support under EY023993 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 26, 2019, is named 117823-19020_SL.txt and is 97,861 bytes in size.

### BACKGROUND OF THE INVENTION

Adeno-associated viruses (AAVs) are small single-stranded DNA viruses in the Parvoviridae family that have several advantages as somatic gene therapy vectors and, thus, have emerged as the vector of choice for degenerative ocular disease, such as inherited retinal diseases and disorders. There are many recessive disease genes associated with inherited retinal diseases and disorders, and complementation by a vector-encoded gene can lead to an improvement in vision (Acland et al., 2001). The target cells for retinal gene therapy are most often the photoreceptors (rods and cones) and retinal pigment epithelial (RPE) cells, as most genetic retinal diseases initiate with dysfunction, often followed by death, of these cell types. There are two types of photoreceptors: rods, necessary for dim light vision, and cones, required for bright light and color vision. Vision initiates with the detection of light in an elaborate and specialized photoreceptor structure, the outer segment (OS), whose morphology can serve as an indicator of photoreceptor health. Photoreceptor cells are supported by the RPE, an epithelial layer with processes in close contact with the photoreceptor OS's. Injections into the subretinal space, the virtual space between the RPE and photoreceptors, is, thus, the injection site for most ocular human gene therapy. In addition to these target cell types being accessible for gene therapy, the eye offers several other advantages for somatic gene therapy. It is relatively immune privileged, anatomically compartmentalized, and can be targeted by established clinical interventions. Its target cells do not replicate and, thus, do not need integrating viruses. One further attribute that is particularly valuable, given the expense of generating pure viral vectors, is that only a small amount of virus is needed for local administration. These advantages stand in contrast to the systemic administration required for large organs, such as liver or muscle, and led to the approval of AAV encoding the RPE65 gene (Luxturna) for Leber's Congenital Amaurosis 2 (LCA2), a rare retinal disease (Bainbridge et al., 2008; Hauswirth et al., 2008; Maguire et al., 2008). AAV has proven to be safe in the LCA2 clinical trials, as well as in several clinical trials for other ocular diseases such as choroideremia and retinitis pigmentosa (Ghazi et al., 2016; MacLaren et al., 2014).

One type of degenerative ocular disease is retinitis pigmentosa (RP) which is a family of retinal degenerations (RD) associated with reduced viability of cone cells that is currently incurable and frequently leads to blindness. Affecting roughly 1 in 3,000 individuals, it is the most prevalent form of RD caused by a single disease allele (RetNet, .uth.edu). The phenotype is characterized by an initial loss of night vision due to the malfunction and death of rod photoreceptors, followed by a progressive loss of cones (Madreperla, S. A., et al. (1990) Arch Ophthalmol 108, 358-61). Additionally, retinitis pigmentosa is further characterized by, e.g., night blindness, progressive loss of peripheral vision, eventually leading to total blindness, ophthalmoscopic changes consisting of dark mosaic-like retinal pigmentation, attenuation of the retinal vessels, waxy pallor of the optic disc, and in the advanced forms, macular degeneration. Since cones are responsible for color and high acuity vision, it is their loss that leads to a reduction in the quality of life. In many cases, the disease-causing allele is expressed exclusively in rods; nonetheless, cone cell death follows rod cell death. Indeed, to date there is no known form of RD in humans or mice where rods die, and cones survive. In contrast, mutations in cone-specific genes result only in cone death.

Accordingly, there is a need in the art for therapies to treat and prevent vision loss that results from degenerative ocular diseases, such as retinitis pigmentosa.

### SUMMARY OF THE INVENTION

The present invention is based, at least in part, on the discovery that intraocular, e.g., subretinal, delivery of some AAVs commonly used as somatic gene therapy vectors for treatment of retinal disorders consistently induced cone OS shortening, reduction of the outer nuclear layer (ONL) where rods and cones reside, and dysmorphic RPE, in mice and that this retinal toxicity is correlated with AAV vector/construct structure.

The present invention is also based, at least in part, of the identification of the critical elements for inclusion in AAV constructs that reduce this toxicity while maintaining pharmacological activity and/or that impart benefits of pharmacological activity to the constructs that outweigh any toxicity associated with the constructs, and the production of such AAV constructs that are therapeutically effective for treating degenerative ocular diseases, such as retinitis pigmentosa.

Accordingly, the present invention provides compositions, e.g., pharmaceutical compositions, which include a recombinant adeno-associated virus (AAV) vector, and methods of treating a subject having a degenerative ocular disorder, e.g., retinitis pigmentosa.

In one aspect, the present invention provides a composition, comprising an adeno-associated virus (AAV) expression cassette, the expression cassette comprising a human bestrophin 1 (hBest1) promoter, a chimeric intron, and a nucleic acid molecule encoding nuclear factor erythroid 2-like 2 (Nrf2).

In one embodiment, the hBest1 promoter comprises nucleotides −585 to +38 of the hBest1gene; nucleotides −154 to +38 of the hBest1 gene; or nucleotides −104 to +38 bp of the hBest1 gene, or or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides −585 to +38 of the hBest1gene; nucleotides −154 to +38 of the hBest1 gene; or nucleotides −104 to +38 bp of the hBest1 gene.

In one embodiment, the hBest1 promoter comprises nucleotides 4885-5507 of SEQ ID NO:9, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4885-5507 of SEQ ID NO:9.

In one embodiment, the nucleic acid molecule encoding Nrf2 comprises the nucleotide sequence selected from the group consisting of the Nrf2 transcript variant 1 (SEQ ID NO:1), the Nrf2 transcript variant 2 (SEQ ID NO:2), the Nrf2 transcript variant 3 (SEQ ID NO:3), the Nrf2 transcript variant 4 (SEQ ID NO:4), the Nrf2 transcript variant 5 (SEQ ID NO:5), the Nrf2 transcript variant 6 (SEQ ID NO:6), the Nrf2 transcript variant 7 (SEQ ID NO:7), and the Nrf2 transcript variant 8 (SEQ ID NO:8), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of any one of the Nrf2 transcript variant 1 (SEQ ID NO:1), the Nrf2 transcript variant 2 (SEQ ID NO:2), the Nrf2 transcript variant 3 (SEQ ID NO:3), the Nrf2 transcript variant 4 (SEQ ID NO:4), the Nrf2 transcript variant 5 (SEQ ID NO:5), the Nrf2 transcript variant 6 (SEQ ID NO:6), the Nrf2 transcript variant 7 (SEQ ID NO:7), or the Nrf2 transcript variant 8 (SEQ ID NO:8).

In one embodiment, the nucleic acid molecule encoding Nrf2 comprises the nucleotide sequence of the Nrf2 transcript variant 1 (SEQ ID NO:1), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:1.

In one embodiment, the nucleic acid molecule encoding Nrf2 comprises the nucleotide sequence selected from the group consisting of the Nrf2 transcript variant 1 (SEQ ID NO:10), the Nrf2 transcript variant 2 (SEQ ID NO:00), the Nrf2 transcript variant 3 (SEQ ID NO:12), the Nrf2 transcript variant 4 (SEQ ID NO:13), the Nrf2 transcript variant 5 (SEQ ID NO:14), the Nrf2 transcript variant 6 (SEQ ID NO:15), the Nrf2 transcript variant 7 (SEQ ID NO:16), and the Nrf2 transcript variant 8 (SEQ ID NO:17), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of any one of the Nrf2 transcript variant 1 (SEQ ID NO:10), the Nrf2 transcript variant 2 (SEQ ID NO:11), the Nrf2 transcript variant 3 (SEQ ID NO:12), the Nrf2 transcript variant 4 (SEQ ID NO:13), the Nrf2 transcript variant 5 (SEQ ID NO:14), the Nrf2 transcript variant 6 (SEQ ID NO:15), the Nrf2 transcript variant 7 (SEQ ID NO:16), or the Nrf2 transcript variant 8 (SEQ ID NO:17).

In one embodiment, the nucleic acid molecule encoding Nrf2 comprises the nucleotide sequence of the Nrf2 transcript variant 1 (SEQ ID NO:10), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:10.

In one embodiment, the chimeric intron comprises a 5′-donor site from the first intron of the human β-globin gene and the branch and 3′-acceptor site from the intron that is between the leader and the body of an immunoglobulin gene heavy chain variable region.

In one embodiment, the chimeric intron comprises nucleotides 1120-1252 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 1120-1252 of the nucleotide sequence in FIG. 19.

In one embodiment, the expression cassette of the invention further comprises a post-transcriptional regulatory region.

In one embodiment, the expression cassette of the invention further comprises a Woodchuck hepatitis virus posttranscriptional regulatory element (WPRE).

In one embodiment, the expression cassette of the invention further comprises a post-transcriptional regulatory region comprising nucleotides 3110-3651 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3110-3651 of the nucleotide sequence in FIG. 19.

In one embodiment, the expression cassette of the invention further comprises a post-transcriptional regulatory region comprising the nucleotide sequence of SEQ ID NO: 18, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 18.

In one embodiment, the expression cassette of the invention is present in a vector.

In one embodiment, the vector is an AAV vector selected from the group consisting of AAV2, AAV 8, AAV2/5, and AAV 2/8.

In one aspect, the present invention provides an AAV vector particle comprising any one of the compositions of the invention.

In one aspect, the present invention provides an isolated cell comprising the AAV vector particle of the invention.

In one aspect, the present invention provides a pharmaceutical composition comprising any one of the AAV compositions of the invention or the AAV vector particle of the invention.

In one embodiment, the pharmaceutical compostion further comprises a viscosity inducing agent.

In one embodiment, the pharmaceutical compostion is for intraocular administration.

In one embodiment, the intraocular administration of the pharmaceutical composition is selected from the group consisting of intravitreal or subretinal, subvitreal, subconjuctival, sub-tenon, periocular, retrobulbar, suprachoroidal, and/or intrascleral administration.

In one aspect, the present invention provides a method for prolonging the viability of a photoreceptor cell compromised by a degenerative ocular disorder, comprising contacting said cell with any one of the compositions of the invention, the AAV viral particle of the invention, or any one of the pharmaceutical compositions of the invention, thereby prolonging the viability of the photoreceptor cell compromised by the degenerative ocular disorder.

In one aspect, the present invention provides a method for treating or preventing a degenerative ocular disorder in a subject, comprising administering to said subject a therapeutically effective amount of any one of the compositions of the invention, the AAV viral particle of the invention, or any one of the pharmaceutical compositions of the invention, thereby treating or preventing said degenerative ocular disorder.

In one aspect, the present invention provides a method for delaying loss of functional vision in a subject having a degenerative ocular disorder, comprising administering to said subject a therapeutically effective amount of any one of the compositions of the invention, the AAV viral particle of the invention, or the any one of the pharmaceutical compositions of the invention, thereby treating or preventing said degenerative ocular disorder.

In one aspect, the present invention provides a method for treating or preventing retinitis pigmentosa in a subject, comprising administering to the subject a therapeutically effective amount of any one of the compositions of the invention, the AAV viral particle of the invention, or any one of the pharmaceutical compositions of the invention, thereby treating or preventing retinitis pigmentosa in said subject.

In one embodiment, the degenerative ocular disorder is associated with decreased viability of cone cells and/or decreased viability of rod cells.

In one embodiment, the degenerative ocular disorder is selected from the group consisting of retinitis pigmentosa, age related macular degeneration, cone rod dystrophy, and rod cone dystrophy.

In one embodiment, the degenerative ocular disorder is a genetic disorder.

In one embodiment, the degenerative ocular disorder is not associated with blood vessel leakage and/or growth.

In one embodiment, the degenerative ocular disorder is retinitis pigmentosa.

Other features and advantages of the invention will be apparent from the following detailed description and claims

### BRIEF DESCRIPTION OF THE DRAWINGS

FIGS. 1A-1C depict that retina toxicity is induced by broadly active promoters but not by retinal cell type-specific promoters. FIGS. 1A and 1B are immunohistochemical images of wild type retinas infected with the indicated viruses at either low (3E9 vp/eye) or high dose (8E12 vp/eye) (1A) or controls (1B) and harvested at P30 for histology. Retinal cross-sections were stained for short- and medium/long-wavelength opsins (dark gray) and for GFAP (light gray) (1A and 1B). Loss of opsin staining and upregulated expression of GFAP were observed in the retinas infected with AAV8-CMV-GFP and AAV8-CMV-null. Scale bar, 100 μm. FIG. 1C are graphs depicting quantification of ONL thickness at 1 mm from optic nerve head (ONH) at either low (3E9 vp/eye; left-hand graph) or high dose (8E12 vp/eye). Data presented as Mean±SD. n=3-17 per group. One-way ANOVA analysis with Tukey test, ** p<0.01; n.s. not significant between the designated group and the uninjected group.

FIGS. 2A and 2B depict that RPE toxicity is induced by broadly active promoters. FIG. 1A are representative microscopic images of the grading criteria of RPE toxicity, with grade 0 (upper left) representing completely healthy RPE and grade 5 (lower right) representing the most severe RPE damage. The typical phenotypes of each grade are described below each image. Scale bar, 50 μm. FIG. 2B is a scatter dot plot of RPE toxicity grades. Data presented as Mean±SD. n=2-8 per group. One-way ANOVA analysis with Tukey test, ** p<0.01; n.s. not significant between the designated group and the uninjected group.

FIGS. 3A-3C depict RPE toxicity is induced by the hBest1 promoter. Representative image of RPE toxicity after AAV8-hBest1-GFP injection (3A) and AAV8-hBest1-mApple (3B). Scale bar, 50 μm. FIG. 3C is a scatter dot plot of RPE toxicity grades. Uninjected and AAV8-hBest1-GFP-injected eyes were harvested on P30, and AAV8-hBest1-mApple-injected eyes were harvested at P72. Data presented as Mean±SD. n=4-5 per group. One-way ANOVA analysis with Tukey test, ** p<0.01.

FIGS. 4A-4H are photographic images depicting that the RPE and retina damage from toxic AAV virus is dose-dependent. Representative images of RPE and retina toxicity resulting from low dose (5E8 vp/eye), medium dose (1E9 vp/eye) and high dose (2E9 vp/eye) of AAV8-CMV-GFP are shown. FIGS. 4A-4C are images of the RPE from retinas infected with the indicated viruses and labeled with phalloidin staining, and FIGS. 4D-4F are images of photoreceptors from retinas infected with the indicated viruses and labeled with peanut agglutinin (PNA) from the same areas as in FIGS. 4A-4C. FIGS. 4G and 4H are images of a retina partially infected with the AAV8-CMV-GFP vector showing photoreceptor toxicity only in the infected area of infection. Magnified views of boxed areas (images between FIGS. 4G and 4H) show loss of outer segments in the infected area.

FIGS. 5A-5G depict that toxic AAV causes OCT, ERG, and optomotor manifestations in C57BL/6J mouse eyes. FIG. 5A are representative micron funduscopic images of AAVs administered at low (abbreviation: L, 8E8 vp/eye) or high (abbreviation: H, 3E9 vp/eye) doses. From left to right are: AAV8-CAR-GFP, AAV8-RedO-GFP (AAV8-hRedO-GFP plus AAV8-Best1-GFP, AAV8-RedO-GFP was injected with AAV8-Best1-GFP at 5:1 ratio in order to match the CMV-driven expression profile by including RPE expression), AAV8-CMV-GFP, and AAV5-CMV-GFP infected mouse eyes (˜P30). White arrow line labels are the plane where OCT was taken. FIG. 5B are representative OCT images of eyes injected with low and high doses of AAV8-CAR-GFP, AAV8-RedO-GFP (plus AAV8-Best1-GFP), AAV8-CMV-GFP, and AAV5-CMV-GFP, respectively. White arrow-head: intrusions in subretinal space. IPL: inner plexiform layer, OPL: outer plexiform layer, ONL: outer nuclear layer, OLM: outer limiting membrane, IS/OS: junction of inner and outer segments of photoreceptors, RPE: retinal pigmented epithelium. FIG. 5C are representative trace of scotopic ERG (flash intensity: 0.1 cd s/m2, wavelength: 530 nm) of eyes injected with AAV8-RedO-GFP (plus AAV8-Best1-GFP) (low dose: black trace, and high dose: gray dashed trace), and AAV8-CMV-GFP (low dose: gray trace, and high dose: light gray dashed trace). FIG. 5D are statistics of scotopic ERG parameters (a-wave and b-wave amplitude and implicit time) of eyes injected with AAV8-RedO-GFP (plus AAV8-Best1-GFP) (control low dose: black circles, n=8; and high dose: gray upper triangles, n=7), and AAV8-CMV-GFP (low dose: gray square, n=10; and high dose: kight gray lower triangles, n=8). FIG. 5E are representative traces of photopic ERG of eyes injected with AAV8-RedO-GFP (plus AAV8-Best1-GFP) (low dose: black trace, and high dose: gray dashed trace), and AAV8-CMV-GFP (low dose: gray trace, and high dose: light gray dashed trace), elicited by 1 (peak), 10 (peak), 100 (Xenon), and 1000 (Xenon) cd s/mz white light flashes with a white light background (bkg) of 30 cd/m2. FIG. 5F are ensemble-averaged photopic ERG b-wave amplitude from eyes injected with AAV8-RedO-GFP (plus AAV8-Best1-GFP) (low dose: black circles, n=8; and high dose: gray upper triangles, n=7), and AAV8-CMV-GFP (low dose: gray square, n=10; and high dose: light gray lower triangles, n=8). Inset is the normalized photopic ERG b-wave intensity (r/rmax)−response curves. FIG. 5G are photopic optomotor responses from eyes injected with AAV8-RedO-GFP (plus AAV8-Best1-GFP) (low dose: (black circles, n=7) and high dose: gray upper triangles, n=7), and AAV8-CMV-GFP (low dose: gray square, n=13; and high dose: light gray lower triangles, n=7). Acuity: tested at 100% contrast; Contrast sensitivity: tested at 0.128 cyc/deg and 1.5 Hz temporal frequency. For all panels in FIG. 5, error bar: SEM, *: p<0.05, ***: p<0.001, ****: p<0.0001***#: p<1×108, NS: not significant.

FIGS. 6A and 6B depict retinal toxicity following injection of AAV5-CMV-GFP at high dose or low dose. FIG. 6A are representative cross-sections of wild-type retinas infected with AAV5-CMV-GFP at either low does (8E8 vg/eye) or high dose (3E9 vg/eye) (˜P30). Control images were selected from uninfected regions with healthy retinal layers of little GFP expression on the same section. Retinal cross-sections were stained for short- and medium/long-wavelength opsins (medium grey). Loss of opsin staining and GFP+ cells were evident in both low and high dose AAV5-CMV-GFP-injected retinas. FIG. 6B are images of Iba-1 staining (medium grey) of retinal sections showing increased number and migration toward ONL of Iba1+microglia in both low and high dose AAV5-CMV-GFP-injected retinas.

FIGS. 7A-7E depict that activation of microglia and innate immune response by toxic AAVs. FIG. 7A is an image of Iba-1 staining of retinal sections infected with the indicated AAV viruses at the dose (3E9 vp/eye). Scale bar: 50 μm. FIG. 7B are images of retinas infected with low dose (8E8 vp/eye) AAV8-CMV-GFP showing displacement of Iba-1 positive cells (arrows) into the ONL. FIG. 7C is a graph depicting quantification of displaced Iba-1 positive cells by cell layer. Values are shown as Mean±SD. n=4 per group. FIG. 7D is a graph depicting quantification of microglia in retinas of P20 Cx3cr1-GFP mice by flow cytometry injected with PBS or 3E8 vp/eye AAV8-CMV-TdTomato (n=3-4 for all groups). FIG. 7E is a graph depicting relative mRNA levels of TNFa, IL-1b, IL-6, and IFNg by qPCR in the retinas infected with the indicated AAV viruses at low (8E8 vp/eye) and high dose (3E9 vp/eye). Expression level was normalized to gapdh. Values are shown as Mean±SEM. n=4-8 per group. One-way ANOVA analysis with Tukey test, ** p<0.01.

FIG. 8 is a Table 1 summarizing retina and RPE toxicity of all viruses tested in this study. Y=toxic, N=not toxic, L=low retina toxicity defined as Iba1+ cell infiltrating to the ONL and GFAP upregulation without ONL thinning. * AAV2 7m8-hBest1-GFP was tested at a low concentration (1E8 vp/eye) due to the low titer of the stock. Y=toxicity, L=low toxicity, N=non-toxic. Toxicity to the retina was assessed by the immunohistological assays shown in FIGS. 1A-C, 4D-F, and 7A and 7C. Toxicity was defined by ONL thinning, loss of S+M/L opsin, GFAP upregulation, and Iba1+ cell infiltration into the ONL, while low toxicity in the retina was defined as Iba1+ cells in the ONL and GFAP upregulation without ONL thinning. Toxicity to the RPE was assessed by the 0-5 grading method on RPE flatmounts shown in FIG. 2A-B, and toxicity was defined as a score >1. Non-toxic means that retina and RPE were indistinguishable from the uninjected controls. All viruses were tested at a dose of 8E8 vg/eye.

FIG. 9 depicts purity of AAV preparations by SDS gel analysis. Lane 1 and 2: reference virus preps. Lane 4 and 5: toxic preps with little or no protein contaminants. Lane 3, 6 and 7: non-toxic preps. Lane 7: non-toxic prep with protein contaminants (arrow).

FIG. 10 are representative images of RPE toxicity resulting from infection with the indicated viruses. Scale bar: 100 μm.

FIG. 11 are depictions of vector maps of the viruses used in the examples.

FIG. 12 are images depicting RPE damage increases with incubation time. Representative images of RPE (labeled with phalloidin) from CD1 mice injected with AAV8-CMV-GFP at 8E8 vp/eye, harvested at the indicated. Toxicity grades for each sample are listed in the table on the right (see FIG. 2 and text).

FIG. 13A are images depicting wild type retinas were injected with AAV8-CMV-GFP (8E8 vp/eye) via either a subretinal or intravitreal route. Subretinally delivered AAV8-CMV-GFP induced retinal toxicity, shown by anti-S+M/L opsin (upper panel) and anti-GFAP (lower panel) staining, while intravitreal delivery of the same virus preparation did not induce any obvious toxicity. SR, subretinal; IV, intravitreal.

FIG. 13B is a graph depicting relative mRNA levels of TNFα, IL-1β, IL-6, and IFNγ by qPCR in retinas infected with AAV8-CMV-GFP (8E8 vp/eye) via either a subretinal or intravitreal route. Expression level was normalized to gapdh. Values shown as Mean±SEM. n=3-4 per group. One-way ANOVA analysis with Tukey test, ** p<0.01.

FIG. 14 schematically depicts exemplary elements of an exemplary expression cassette of the invention.

FIG. 15 depicts an exemplary vector map of an exemplary AAV vector of the invention.

FIG. 16 schematically depicts the construction of an exemplary vector comprising Cap and Rep genes for use in producing AAV viral particles of the invention.

FIG. 17 schematically depicts a helper vector for use in producing AAV vector particles of the invention.

FIG. 18A is a graph depicting the effect of the indicated amounts of AAV viral genome comprising a composition of the invention on visual behavior as determined by optomotor response.

FIG. 18B is a graph depicting the effect of the indicated amounts of AAV viral genome comprising a composition of the invention on cone function as determined by photopic ERG b wave.

FIG. 18C is a graph depicting the effect of the indicated amounts of AAV viral genome comprising a composition of the invention on cone marker as determined by opsin count.

FIGS. 19A-19J depict the nucleotide sequence of the exemplary vector map of an exemplary AAV vector of the invention depicted in FIG. 15. FIGS. 19A-19J disclose SEQ ID NO: 21 as the full-length sequence, protein sequences as SEQ ID NOS 22-24, and primers as SEQ ID NOS 25 and 26, respectively, in order of appearance.

### DETAILED DESCRIPTION OF THE INVENTION

The present invention is based, at least in part, on the discovery that intraocular, e.g., subretinal, delivery of some AAVs commonly used as somatic gene therapy vectors for treatment of retinal disorders consistently induced cone OS shortening, reduction of the outer nuclear layer (ONL) where rods and cones reside, and dysmorphic RPE, in mice and that this toxicity is correlated with AAV vector/construct structure.

The present invention is also based, at least in part, of the identification of the elements for inclusion in AAV constructs that reduce this toxicity while maintaining pharmacological activity and/or that impart benefits of pharmacological activity to the constructs that outweigh any toxicity associated with the constructs and the production of such AAV constructs that are therapeutically effective for treating degenerative ocular diseases, such as retinitis pigmentosa.

Accordingly, the present invention provides compositions, e.g., pharmaceutical compositions, comprising a recombinant adeno-associated virial vector and methods of treating a subject having a degenerative ocular disease or disorder, e.g., retinitis pigmentosa.

Various aspects of the invention are described in further detail in the following subsections:

### I. Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. A nucleic acid molecule used in the methods of the present invention can be isolated using standard molecular biology techniques. Using all or portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

An “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived.

A nucleic acid molecule for use in the methods of the invention can also be isolated by the polymerase chain reaction (PCR) using synthetic oligonucleotide primers designed based upon the sequence of a nucleic acid molecule of interest. A nucleic acid molecule used in the methods of the invention can be amplified using cDNA, mRNA or, alternatively, genomic DNA as a template and appropriate oligonucleotide primers according to standard PCR amplification techniques. Furthermore, oligonucleotides corresponding to nucleotide sequences of interest can be prepared by standard synthetic techniques, e.g., using an automated DNA synthesizer.

The nucleic acids for use in the methods of the invention can also be prepared, e.g., by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes or nucleic acid molecules to which they are operatively linked and are referred to as “expression vectors” or “recombinant expression vectors.”. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

Expression vectors are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses, adeno-associated viruses, lentiviruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells, those which are constitutively active, those which are inducible, and those which direct expression of the nucleotide sequence only in certain host cells (e.g., tissue-specific regulatory sequences). The expression vectors of the invention can be introduced into host cells to thereby produce proteins or portions thereof, including fusion proteins or portions thereof, encoded by nucleic acids as described herein.

The terms “transformation,” “transfection,” and “transduction” refer to introduction of a nucleic acid, e.g., a viral vector, into a recipient cell.

As used herein, the term “subject” includes warm-blooded animals, preferably mammals, including humans. In a preferred embodiment, the subject is a primate. In an even more preferred embodiment, the primate is a human.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (i.e., contacting a cell with an agent) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

As used herein, the term “administering” to a subject includes dispensing, delivering or applying a composition of the invention to a subject by any suitable route for delivery of the composition to the desired location in the subject, including delivery by intraocular administration or intravenous administration. Alternatively or in combination, delivery is by the topical, parenteral or oral route, intracerebral injection, intramuscular injection, subcutaneous/intradermal injection, intravenous injection, buccal administration, transdermal delivery and administration by the rectal, colonic, vaginal, intranasal or respiratory tract route.

As used herein, the term “degenerative ocular disorder” refers generally to a disorder of the retina. In one embodiment, the degenerative ocular disorder is associated with death, of cone cells, and/or rod cells. Moreover, in a particular embodiment, a degenerative ocular disorder is not associated with blood vessel leakage and/or growth, for example, as is the case with diabetic retinopathy, but, instead is characterized primarily by reduced viability of cone cells and/or rod cells. In certain embodiments, the degenerative ocular disorder is a genetic or inherited disorder. In a particular embodiment, the degenerative ocular disorder is retinitis pigmentosa. In another embodiment, the degenerative ocular disorder is age-related macular degeneration. In another embodiment, the degenerative ocular disorder is cone-rod dystrophy. In another embodiment, the degenerative ocular disorder is rod-cone dystrophy. In other embodiments, the degenerative ocular disorder is not associated with blood vessel leakage and/or growth. In certain embodiments, the degenerative ocular disorder is not associated with diabetes and/or diabetic retinopathy. In further embodiments, the degenerative ocular disorder is not NARP (neuropathy, ataxia, and retinitis pigmentosa). In yet further embodiments, the degenerative ocular disorder is not a neurological disorder. In certain embodiments, the degenerative ocular disorder is not a disorder that is associated with a compromised optic nerve and/or disorders of the brain. In the foregoing embodiments, the degenerative ocular disorder is associated with a compromised photoreceptor cell, and is not a neurological disorder.

As used herein, the term “retinitis pigmentosa” or “RP” is known in the art and encompasses a disparate group of genetic disorders of rods and cones. Retinitis pigmentosa generally refers to retinal degeneration often characterized by the following manifestations: night blindness, progressive loss of peripheral vision, eventually leading to total blindness; ophthalmoscopic changes consist in dark mosaic-like retinal pigmentation, attenuation of the retinal vessels, waxy pallor of the optic disc, and in the advanced forms, macular degeneration. In some cases there can be a lack of pigmentation. Retinitis pigmentosa can be associated to degenerative opacity of the vitreous body, and cataract. Family history is prominent in retinitis pigmentosa; the pattern of inheritance may be autosomal recessive, autosomal dominant, or X-linked; the autosomal recessive form is the most common and can occur sporadically.

As used herein, the terms “Cone-Rod Dystrophy” or “CRD” and “Rod-Cone Dystrophy” or “RCD” refer to art recognized inherited progressive diseases that cause deterioration of the cone and rod photoreceptor cells and often result in blindness. CRD is characterized by reduced viability or death of cone cells followed by reduced viability or death of rod cells. By contrast, RCD is characterized by reduced viability or death of rod cells followed by reduced viability or death of cone cells.

As used herein, the term “age-related macular degeneration” also referred to as “macular degeneration” or “AMD”, refers to the art recognized pathological condition which causes blindness amongst elderly individuals. Age related macular degeneration includes both wet and dry forms of AMD. The dry form of AMD, which accounts for about 90 percent of all cases, is also known as atrophic, nonexudative, or drusenoid (age-related) macular degeneration. With the dry form of AMD, drusen typically accumulate in the retinal pigment epithelium (RPE) tissue beneath/within the Bruch's membrane. Vision loss can then occur when drusen interfere with the function of photoreceptors in the macula. The dry form of AMD results in the gradual loss of vision over many years. The dry form of AMD can lead to the wet form of AMD. The wet form of AMD, also known as exudative or neovascular (age-related) macular degeneration, can progress rapidly and cause severe damage to central vision. The macular dystrophies include Stargardt Disease, also known as Stargardt Macular Dystrophy or Fundus Flavimaculatus, which is the most frequently encountered juvenile onset form of macular dystrophy.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

As used herein, the terms “treating” or “treatment” refer to a beneficial or desired result including, but not limited to, alleviation or amelioration of one or more symptoms, diminishing the extent of infection, stabilized (i.e., not worsening) state of infection, amelioration or palliation of the infectious state, whether detectable or undetectable. “Treatment” can also mean prolonging survival as compared to expected survival in the absence of treatment.

Various additional aspects of the methods of the invention are described in further detail in the following subsections.

### II. Compositions of the Invention

The present invention provides adeno-associated viral (AAV) expression cassettes, AAV expression cassettes present in AAV vectors, and AAV vectors comprising a recombinant viral genome which include an expression cassette. Accordingly, in one aspect the present invention provides compositions comprising an adeno-associated virus (AAV) expression cassette, the expression cassette comprising a human bestrophin 1 (hBest1) promoter, a chimeric intron, and a nucleic acid molecule encoding nuclear factor erythroid 2-like 2 (Nrf2).

In some embodiments of the invention, the expression cassettes of the invention further comprise expression control sequences including, but not limited to, appropriate transcription sequences (i.e. initiation, termination, and enhancer), efficient RNA processing signals (e.g. splicing and polyadenylation (polyA) signals), sequences that stabilize cytoplasmic mRNA, sequences that enhance translation efficiency (i.e. Kozak consensus sequence), sequences that enhance protein stability, and when desired, sequences that enhance secretion of the encoded product.

The terms “adeno-associated virus”, “AAV virus”, “AAV virion”, “AAV viral particle”, and “AAV particle”, as used interchangeably herein, refer to a viral particle composed of at least one AAV capsid protein (preferably by all of the capsid proteins of a particular AAV serotype) and an encapsidated polynucleotide AAV genome. If the particle comprises a heterologous polynucleotide (i.e. a polynucleotide other than a wild-type AAV genome such as a transgene to be delivered to a mammalian cell) flanked by the AAV inverted terminal repeats (ITRs), it is typically referred to as an “AAV vector particle.”

AAV viruses belonging to the genus Dependovirus of the Parvoviridae family and, as used herein, include any serotype of the over 100 serotypes of AAV viruses known. In general, serotypes of AAV viruses have genomic sequences with a significant homology at the level of amino acids and nucleic acids, provide an identical series of genetic functions, produce virions that are essentially equivalent in physical and functional terms, and replicate and assemble through practically identical mechanisms.

The AAV genome is approximately 4.7 kilobases long and is composed of single-stranded deoxyribonucleic acid (ssDNA) which may be either positive- or negative-sensed. The genome comprises inverted terminal repeats (ITRs) at both ends of the DNA strand, and two open reading frames (ORFs): rep and cap. The rep frame is made of four overlapping genes encoding Rep proteins required for the AAV life cycle. The cap frame contains overlapping nucleotide sequences of capsid proteins: VP1, VP2 and VP3, which interact together to form a capsid of an icosahedral symmetry. See Carter B, Adeno-associated virus and adeno-associated virus vectors for gene delivery, Lassie D, et ah, Eds., “Gene Therapy: Therapeutic Mechanisms and Strategies” (Marcel Dekker, Inc., New York, N.Y., US, 2000) and Gao G, et al, J. Virol. 2004; 78(12):6381-6388.

The term “AAV vector” or “AAV construct” refers to a vector derived from an adeno-associated virus serotype, including without limitation, AAV-1, AAV-2, AAV-3, AAV-4, AAV-5, AAV6, AAV7, AAV8, and AAV9. “AAV vector” refers to a vector that includes AAV nucleotide sequences as well as heterologous nucleotide sequences. AAV vectors require only the 145 base terminal repeats in cis to generate virus. All other viral sequences are dispensable and may be supplied in trans (Muzyczka (1992) Curr. Topics Microbiol. Immunol. 158:97-129). Typically, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging.

In particular embodiments, the AAV vector is an AAV2/5 or AAV2/8 vector. Suitable AAV vectors are described in, for example, U.S. Pat. No. 7,056,502 and Yan et al. (2002) J. Virology 76(5):2043-2053, the entire contents of which are incorporated herein by reference.

Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

The term “cap gene” or “AAV cap gene”, as used herein, refers to a gene that encodes a Cap protein. The term “Cap protein”, as used herein, refers to a polypeptide having at least one functional activity of a native AAV Cap protein (e.g. VP1, VP2, VP3). Examples of functional activities of Cap proteins (e.g. VP1, VP2, VP3) include the ability to induce formation of a capsid, facilitate accumulation of single-stranded DNA, facilitate AAV DNA packaging into capsids (i.e. encapsidation), bind to cellular receptors, and facilitate entry of the virion into host.

The term “capsid”, as used herein, refers to the structure in which the viral genome is packaged. A capsid consists of several oligomeric structural subunits made of proteins. For instance, AAV have an icosahedral capsid formed by the interaction of three capsid proteins: VP1, VP2 and VP3.

The term “genes providing helper functions”, as used herein, refers to genes encoding polypeptides which perform functions upon which AAV is dependent for replication (i.e. “helper functions”). The helper functions include those functions required for AAV replication including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, UL8, UL52, and UL29, and herpesvirus polymerase. In one embodiment, a helper function does not include adenovirus E1.

The term “rep gene” or “AAV rep gene”, as used herein, refers to a gene that encodes a Rep protein. The term “Rep protein”, as used herein, refers to a polypeptide having at least one functional activity of a native AAV Rep protein (e.g. Rep 40, 52, 68, 78). A “functional activity” of a Rep protein (e.g. Rep 40, 52, 68, 78) is any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome.

The term “adeno-associated virus ITRs” or “AAV ITRs”, as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to its self-priming which allows the primase-independent synthesis of the second DNA strand. The ITRs have also shown to be required for efficient encapsidation of the AAV DNA combined with generation of fully assembled, deoxyribonuclease-resistant AAV particles.

The term “expression cassette”, as used herein, refers to a nucleic acid construct, generated recombinantly or synthetically, with a series of specified nucleic acid elements, which permit transcription of a particular nucleic acid in a target cell.

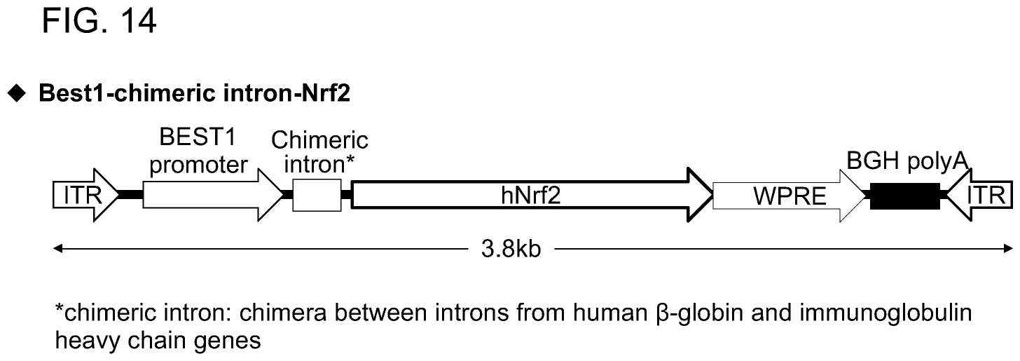

The expression cassettes of the invention include a human bestrophin-1 promoter (hBest1), a chimeric intron, and a nucleic acid molecule encoding nuclear factor erythroid 2-like 2 (Nrf2). An exemplary expression cassette of the invention is depicted in FIG. 14.

The term “promoter” as used herein refers to a recognition site of a DNA strand to which the RNA polymerase binds. The promoter forms an initiation complex with RNA polymerase to initiate and drive transcriptional activity. The complex can be modified by activating sequences termed “enhancers” or inhibitory sequences termed “silencers”.

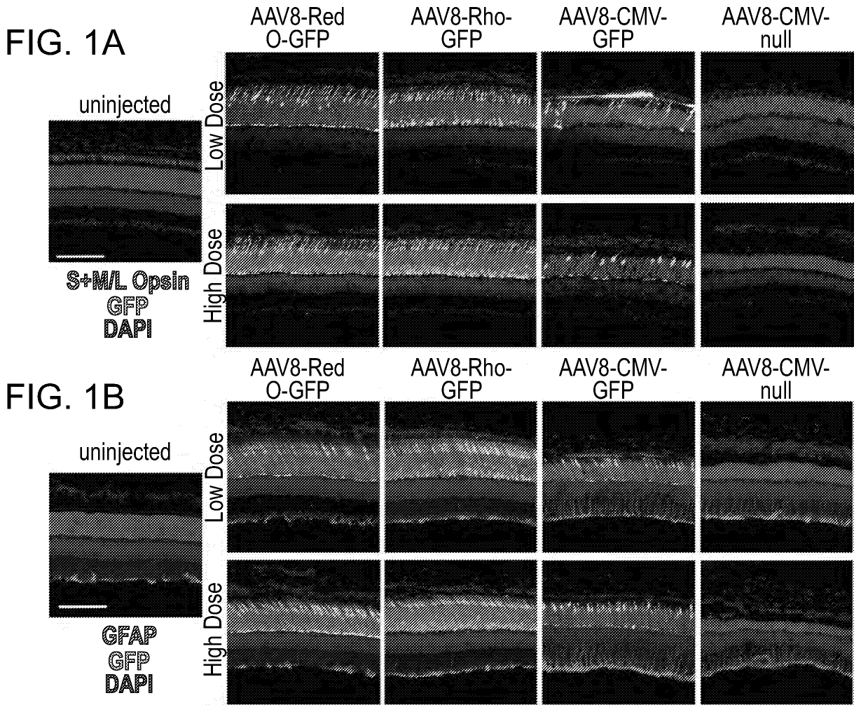

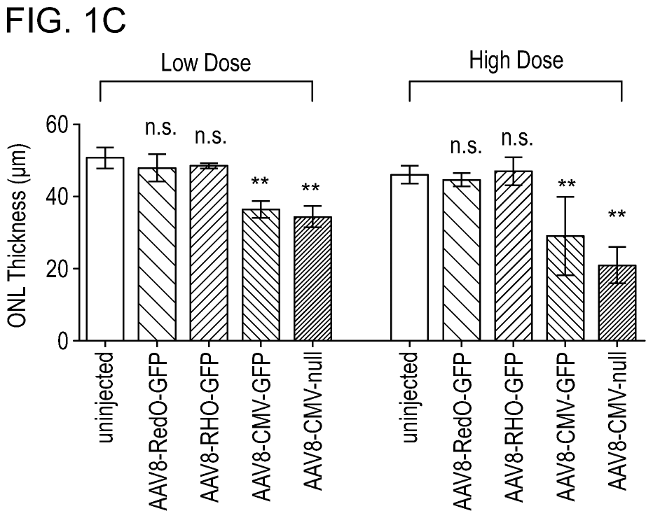

As used interchangeably herein, the terms “bestrophin 1,” “hBest1,” and “hBEST1” refer to bestrophin-1, also known as Bestrophin 1; Vitelliform Macular Dystrophy Protein 2; Best Disease; TU15B; VMD2; Vitelliform Macular Dystrophy 2; Best1V1Delta2; Bestrophin-1; BEST; RP50; ARB; and BMD refers to the gene that is highly and preferentially expressed in the RPE. There are four transcript variants of hBest, the nucleotide and amino acid sequences of which are known and may be found in, for example, GenBank Reference Sequences NM_001139443.1; NM_001300786.1; NM_001300787.1; and NM_004183.3. The nucleotide sequence of the genomic region containing the hBest1 gene (including the region upstream of the coding region of hBest1 which includes the hBest1 promoter region) is also known and may be found in, for example, GenBank Reference Sequence NG_009033.1 (SEQ ID NO: 9, the entire contents of which is incorporated herein by reference; also see, Esumi et al 2004, FIG. 1b at page 19066).

Suitable hBest1 promoters for use in the present invention include nucleic acid molecules which include nucleotides −585 to +38 of the hBest1 gene, (i.e., nucleotides 4885-5507 of SEQ ID NO:9); nucleotides −154 to +38 of the hBest1 gene (i.e., nucleotides 5316-5507 of SEQ ID NO:9); or nucleotides −104 to +38 bp of the hBest1 gene (i.e., nucleotides 5366-5507 of SEQ ID NO:9), or or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4885-5507 of SEQ ID NO:9, nucleotides 5316-5507 of SEQ ID NO:9, or nucleotides 5366-5507 of SEQ ID NO:9. In one embodiment, an hBest1 promoter comprises nucleotides −585 to +38 of the hBest1gene, (i.e., nucleotides 4885-5507 of SEQ ID NO:9), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4885-5507 of SEQ ID NO:9.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions total # of positions (e.g., overlapping positions)×100).

The determination of percent identity between two sequences may be accomplished using a mathematical algorithm. A non-limiting example of a mathematical algorithm utilized for the comparison of two sequences is the algorithm of Karlin and Altschul (1990) Proc. Natl. Acad. Sol. USA 87:2264-2268, modified as in Karlin and Altschul (1993) Proc. Nati. Accid Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score—50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res 25:3389-3402, which is able to perform gapped local alignments for the programs BLASTN, BLASTP and BLASTX.

As used herein, the term “Nrf2” or “NRF2” refers to nuclear factor (erythroid-derived 2)-like 2 (nrf2), a transcription factor which is a member of a small family of basic leucine zipper (bZIP) proteins. Nrf2 is also known as Nuclear Factor, Erythroid 2 Like 2; NF-E2-Related Factor 2; HEBP1, Nuclear Factor Erythroid 2-Related Factor 2; Nuclear Factor, Erythroid Derived 2, Like 2; Nuclear Factor (Erythroid-Derived 2)-Like 2; Nuclear Factor Erythroid-Derived 2-Like 2; Nuclear Factor, Erythroid 2-Like 2; NFE2-Related Factor 2; and IMDDHH. The encoded transcription factor regulates genes which contain antioxidant response elements (ARE) in their promoters. There are eight transcript variants of Nrf2, the nucleotide and amino acid sequences of which are known and may be found in, for example, GenBank Reference Sequences NM_006164.4; NM_001145412.3; NM_001145413.3; NM_001313900.1; NM_001313901.1; NM_001313902.1; NM_001313903.1; and NM_001313904.1 (SEQ ID NOs:1-8, respectively). In one embodiment, a nucleic acid molecule encoding Nrf2 comprises the nucleotide sequence selected from the group consisting of the Nrf2 transcript variant 1 (SEQ ID NO:1), the Nrf2 transcript variant 2 (SEQ ID NO:2), the Nrf2 transcript variant 3 (SEQ ID NO:3), the Nrf2 transcript variant 4 (SEQ ID NO:4), the Nrf2 transcript variant 5 (SEQ ID NO:5), the Nrf2 transcript variant 6 (SEQ ID NO:6), the Nrf2 transcript variant 7 (SEQ ID NO:7), and the Nrf2 transcript variant 8 (SEQ ID NO:8), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of any one of the Nrf2 transcript variant 1 (SEQ ID NO:1), the Nrf2 transcript variant 2 (SEQ ID NO:2), the Nrf2 transcript variant 3 (SEQ ID NO:3), the Nrf2 transcript variant 4 (SEQ ID NO:4), the Nrf2 transcript variant 5 (SEQ ID NO:5), the Nrf2 transcript variant 6 (SEQ ID NO:6), the Nrf2 transcript variant 7 (SEQ ID NO:7), or the Nrf2 transcript variant 8 (SEQ ID NO:8). In one embodiment, a nucleic acid molecule encoding Nrf2 comprises the nucleotide sequence of the Nrf2 transcript variant 1 (SEQ ID NO:1), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:1. In one embodiment, a nucleic acid molecule encoding Nrf2 comprises the nucleotide sequence selected from the group consisting of the Nrf2 transcript variant 1 (SEQ ID NO:10), the Nrf2 transcript variant 2 (SEQ ID NO:11), the Nrf2 transcript variant 3 (SEQ ID NO:12), the Nrf2 transcript variant 4 (SEQ ID NO:13), the Nrf2 transcript variant 5 (SEQ ID NO:14), the Nrf2 transcript variant 6 (SEQ ID NO:15), the Nrf2 transcript variant 7 (SEQ ID NO:16), and the Nrf2 transcript variant 8 (SEQ ID NO:17), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of any one of the Nrf2 transcript variant 1 (SEQ ID NO:10), the Nrf2 transcript variant 2 (SEQ ID NO:11), the Nrf2 transcript variant 3 (SEQ ID NO:12), the Nrf2 transcript variant 4 (SEQ ID NO:13), the Nrf2 transcript variant 5 (SEQ ID NO:14), the Nrf2 transcript variant 6 (SEQ ID NO:15), the Nrf2 transcript variant 7 (SEQ ID NO:16), or the Nrf2 transcript variant 8 (SEQ ID NO:17). In one embodiment, a nucleic acid molecule encoding Nrf2 comprises the nucleotide sequence of the Nrf2 transcript variant 1 (SEQ ID NO:10), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:10.

The invention further encompasses nucleic acid molecules that differ, due to degeneracy of the genetic code, from the nucleotide sequence of nucleic acids encoding a Nrf2 polypeptide, and thus encode the same protein.

As used herein, “an intron” refers to a non-coding nucleic acid molecule which is removed by RNA splicing during maturation of a final RNA product. A “chimeric intron” is an artificial (or non-naturally occurring intron that enhances mRNA processing and increases expression levels of a downstream open reading frame.

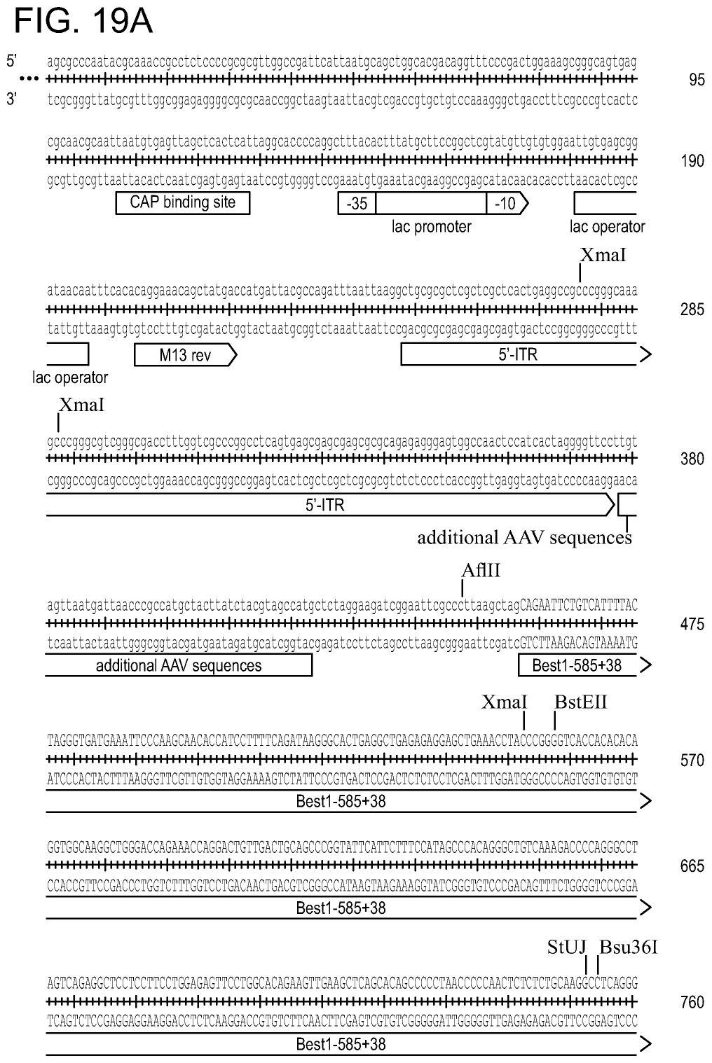

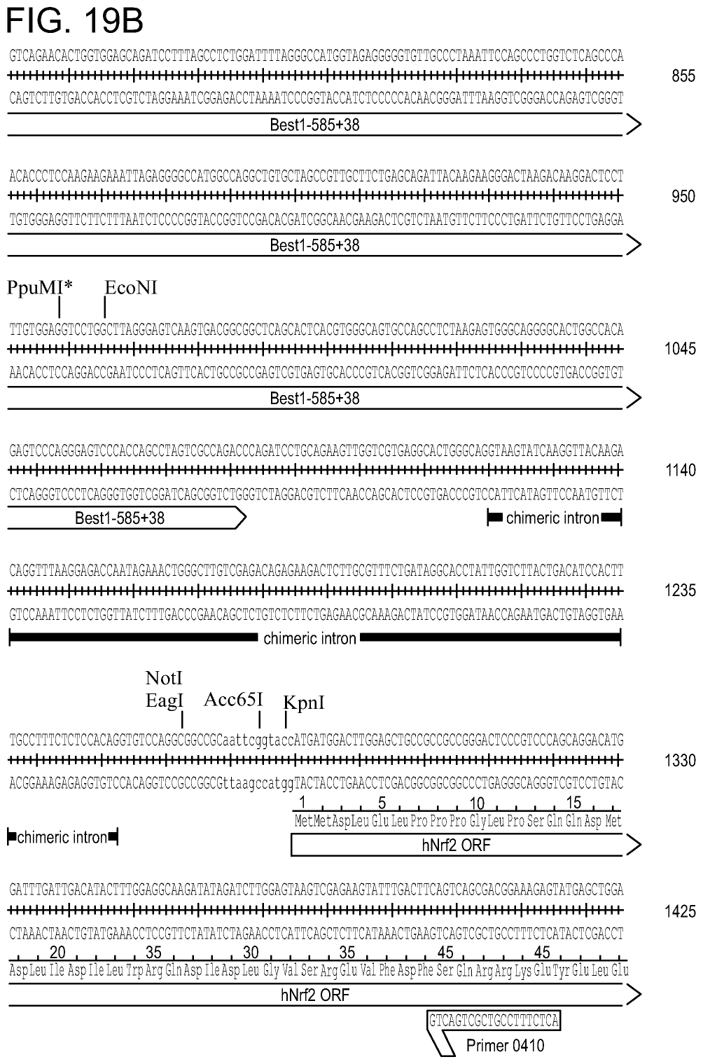

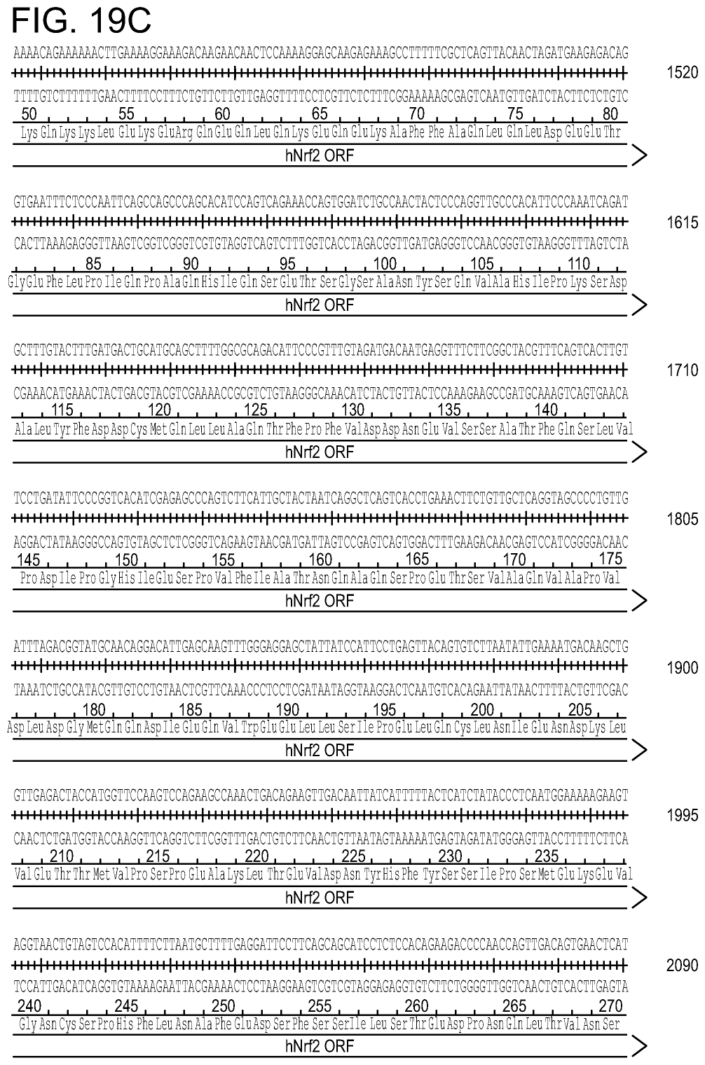

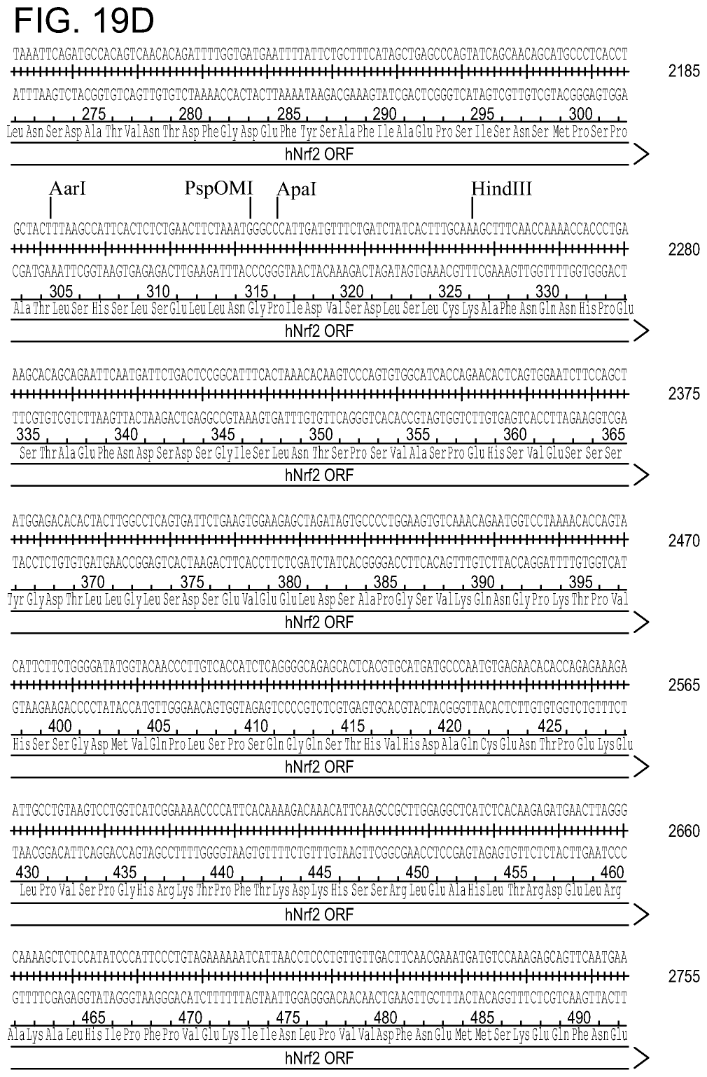

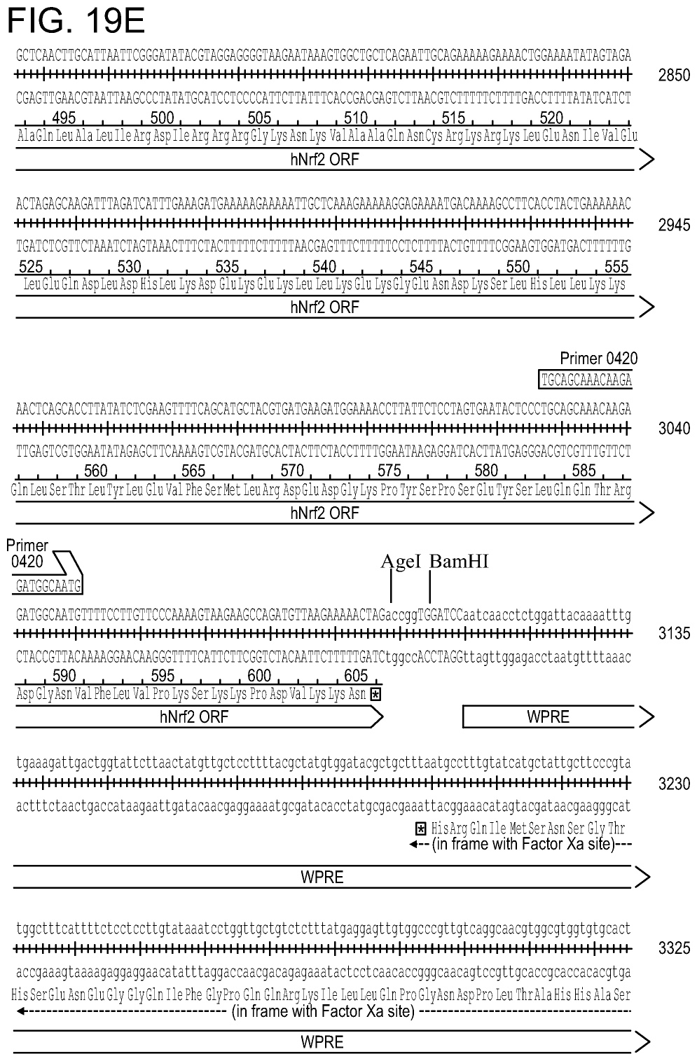

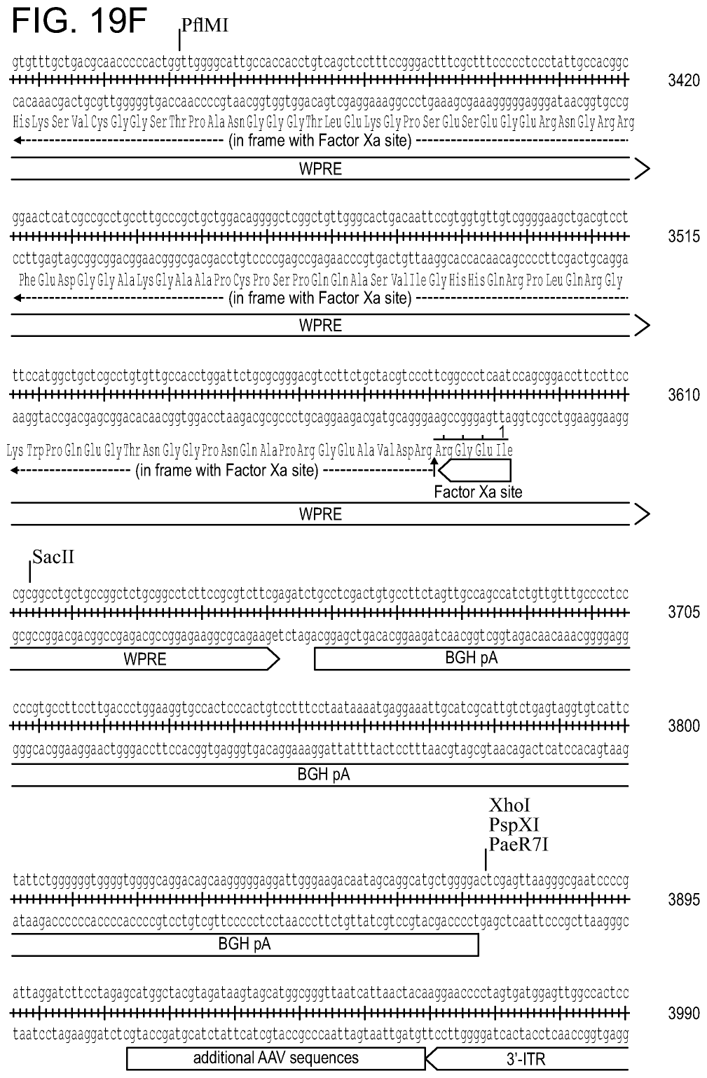

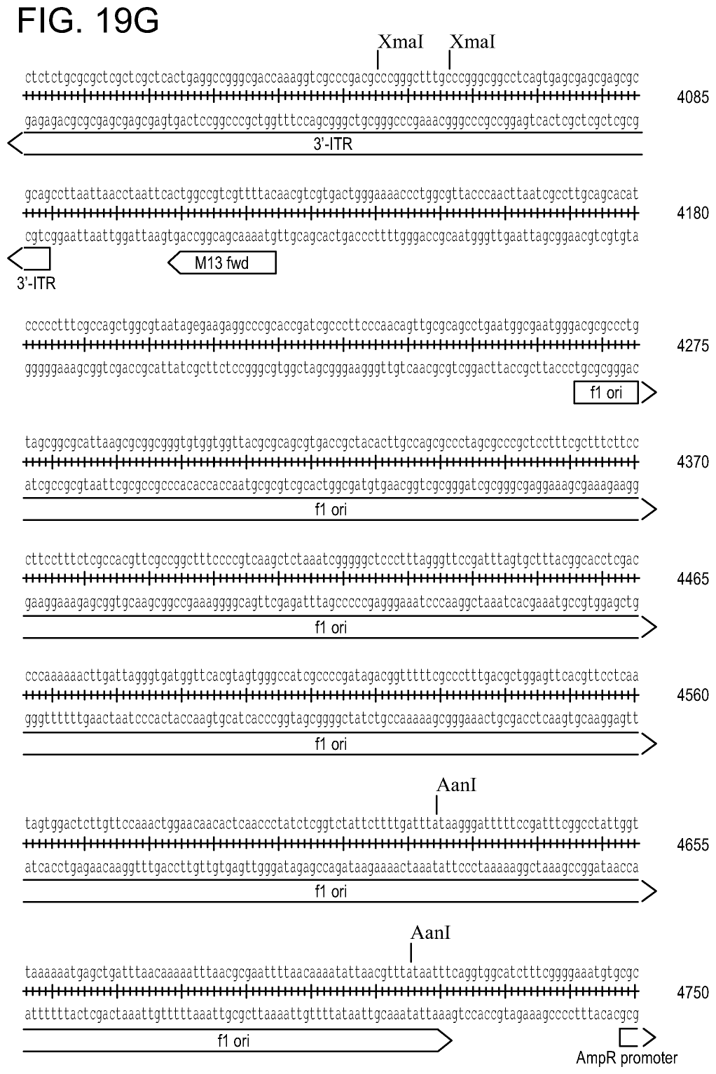

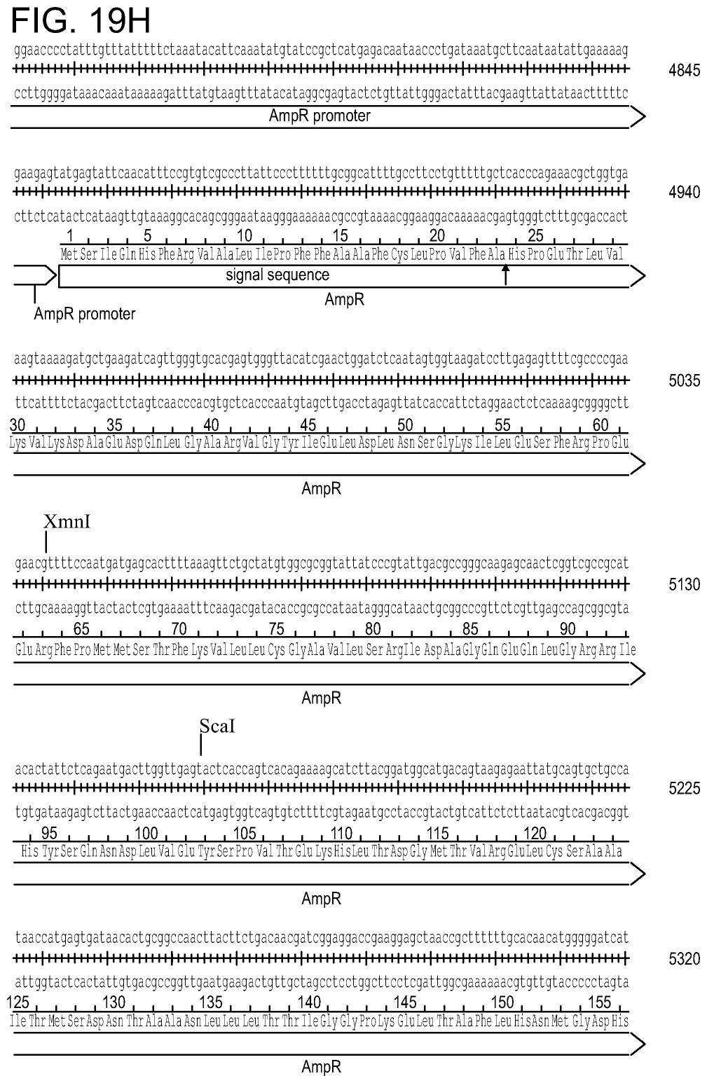

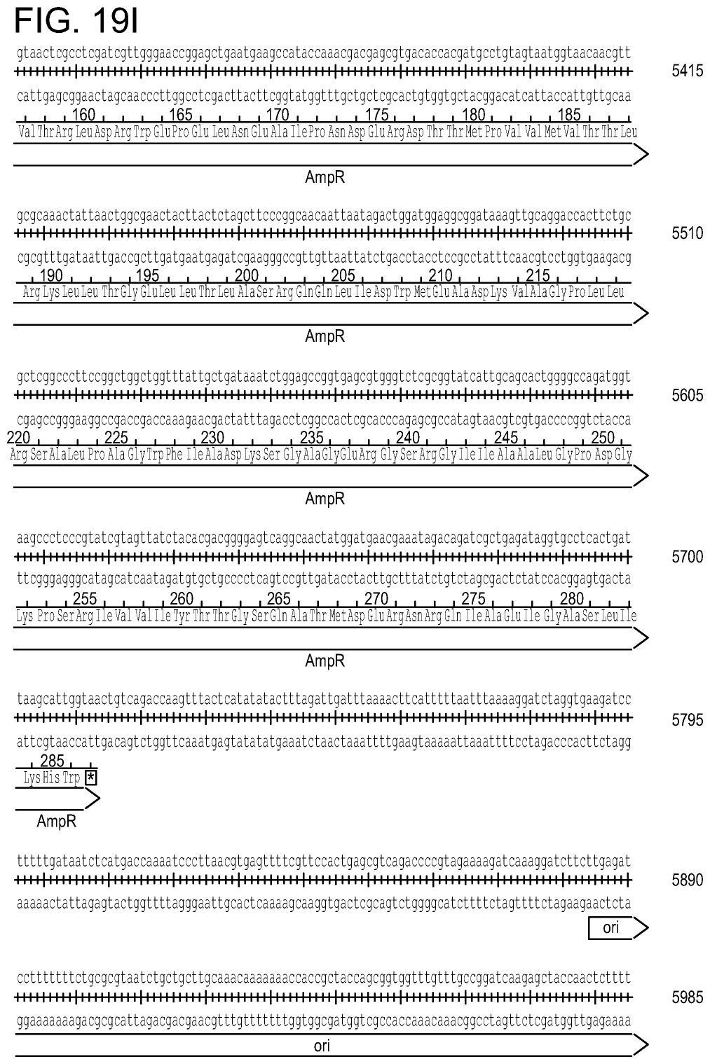

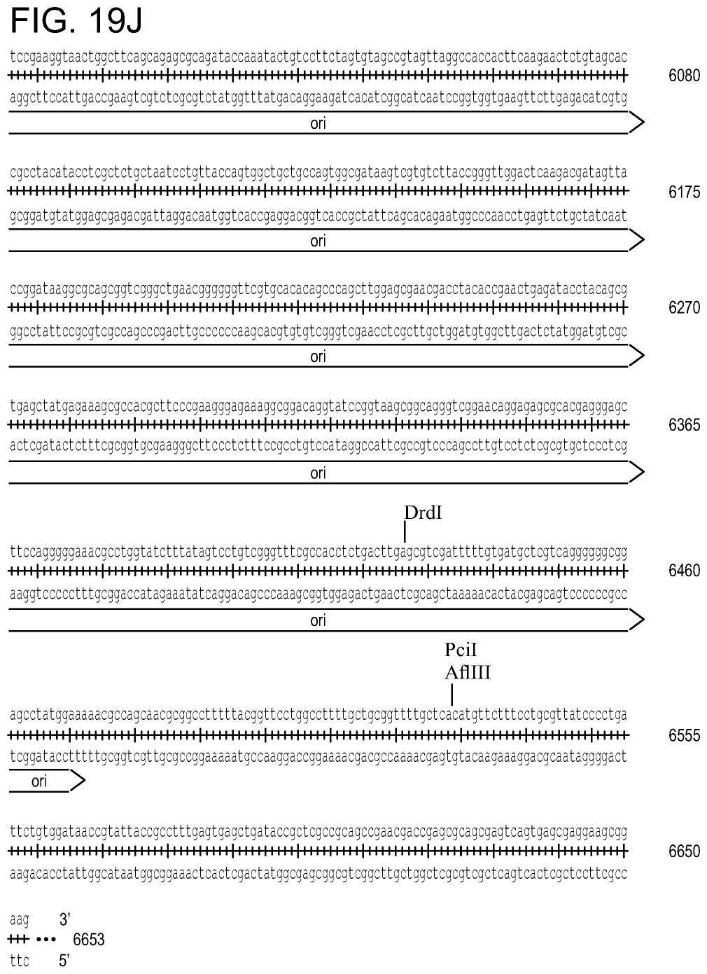

In the expression constructs of the invention, a chimeric intron is flanked by the hBest1 promoter and the nucleic acid molecule encoding Nrf2 and includes the 5′-donor site from the first intron of the human β-globin gene and the branch and 3′-acceptor site from the intron that is between the leader and the body of an immunoglobulin gene heavy chain variable region. In one embodiment, a chimeric intron includes nucleotides 1120-1252 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 1120-1252 of the nucleotide sequence in FIG. 19.

In some embodiments, the expression cassettes of the invention further comprise a post-transcriptional regulatory region.

The term “post-transcriptional regulatory region”, as used herein, refers to any polynucleotide that facilitates the expression, stabilization, or localization of the sequences contained in the cassette or the resulting gene product.

In one embodiment, a post-transcriptional regulatory region suitable for use in the expression cassettes of the invention includes a Woodchuck hepatitis virus post-transcriptional regulatory element.

As used herein, the term “Woodchuck hepatitis virus posttranscriptional regulatory element” or “WPRE,” refers to a DNA sequence that, when transcribed, creates a tertiary structure capable of enhancing the expression of a gene. See Lee Y, et al, Exp. Physiol. 2005; 90(1):33-37 and Donello J, et al, J. Virol. 1998; 72(6):5085-5092.

In one embodiment, a WPRE includes nucleotides 3110-3651 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3110-3651 of the nucleotide sequence in FIG. 19.

In one embodiment, a WPRE includes the nucleotide sequence of SEQ ID NO: 18 (See, e.g., J Virol. 1998 June; 72(6): 5085-5092), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 18.

In some embodiments, the expression cassettes of the invention further comprises a polyadenylation signal.

As used herein, a “polyadenylation signal” or “polyA signal,” as used herein refers to a nucleotide sequence that terminates transcription. Suitable polyadenylation signals for use in the AAV vectors of the invention are known in the art and include, for example, a bovine growth hormone polyA signal (BGH pA). In one embodiment, a BGH pA comprises nucleotides 3658-3872 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3658-3872 of the nucleotide sequence in FIG. 19.

In some embodiments, the expression cassettes of the invention further comprise an enhancer.

The term “enhancer”, as used herein, refers to a DNA sequence element to which transcription factors bind to increase gene transcription.

The AAV vectors of the invention may also include cis-acting 5′ and 3′ inverted terminal repeat (ITR) sequences. In some embodiments, the ITR sequences are about 145 bp in length. In some embodiments, substantially the entire sequences encoding the ITRs are used in the molecule. In other embodiments, the ITRs include modifications. Procedures for modifying these ITR sequences are known in the art. See Brown T, “Gene Cloning” (Chapman & Hall, London, G B, 1995), Watson R, et al, “Recombinant DNA”, 2nd Ed. (Scientific American Books, New York, N.Y., US, 1992), Alberts B, et al, “Molecular Biology of the Cell” (Garland Publishing Inc., New York, N.Y., US, 2008), Innis M, et al, Eds., “PCR Protocols. A Guide to Methods and Applications” (Academic Press Inc., San Diego, Calif., US, 1990), Erlich H, Ed., “PCR Technology. Principles and Applications for DNA Amplification” (Stockton Press, New York, N.Y., US, 1989), Sambrook J, et al, “Molecular Cloning. A Laboratory Manual” (Cold Spring Harbor Laboratory Press, Cold Spring Harbor, N.Y., US, 1989), Bishop T, et al, “Nucleic Acid and Protein Sequence. A Practical Approach” (IRL Press, Oxford, G B, 1987), Reznikoff W, Ed., “Maximizing Gene Expression” (Butterworths Publishers, Stoneham, Mass., US, 1987), Davis L, et al, “Basic Methods in Molecular Biology” (Elsevier Science Publishing Co., New York, N.Y., US, 1986), and Schleef M, Ed., “Plasmid for Therapy and Vaccination” (Wiley-VCH Verlag GmbH, Weinheim, D E, 2001).

The AAV vectors of the invention may include ITR nucleotide sequences derived from any one of the AAV serotypes. In a preferred embodiment, the AAV vector comprises 5′ and 3′ AAV ITRs. In one embodiment, the 5′ and 3′ AAV ITRs derive from AAV2. AAV ITRs for use in the AAV vectors of the invention need not have a wild-type nucleotide sequence (See Kotin, Hum. Gene Ther., 1994, 5:793-801). As long as ITR sequences function as intended for the rescue, replication and packaging of the AAV virion, the ITRs may be altered by the insertion, deletion or substitution of nucleotides or the ITRs may be derived from any of several AAV serotypes or its mutations.

In one embodiment, a 5′ ITR includes nucleotides 248-377 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 248-377 of the nucleotide sequence in FIG. 19.

In one embodiment, a 3′ ITR includes nucleotides 3969-4089 of the nucleotide sequence in FIG. 19, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3969-4089 of the nucleotide sequence in FIG. 19.

In one embodiment, a 5′ ITR includes nucleotides shown by SEQ ID NO: 19 and a 3′ ITR includes nucleotides shown by SEQ ID NO: 20.

In addition, an AAV vector can contain one or more selectable or screenable marker genes for initially isolating, identifying, or tracking host cells that contain DNA encoding the ithe AAV vector (and/or rep, cap and/helper genes) as described herein.

As indicated above, the AAV vectors of the invention may be packaged into AAV viral particles for use in the methods, e.g., gene therapy methods, of the invention (discussed below) to produce AAVvector particles using methods known in the art.

Such methods generally include packaging the AAV vectors of the invention into infectious AAV viral particles in a host cell that has been transfected with a vector encoding and expressing rep and cap gene products (i.e. AAV Rep and Cap proteins), and with a vector which encodes and expresses a protein from the adenovirus open reading frame E4orf6.

Suitable AAV Caps may be derived from any serotype. In one embodiment, the capsid is derived from the AAV of the group consisting on AAV1, AAV2, AAV3, AAV4, AAV5, AAV6, AAV7, AAV8 and AAV9. In another embodiment, the AAV of the invention comprises a capsid derived from the AAV5 or AAV8 serotypes.

In some embodiments, an AAV Cap for use in the method of the invention can be generated by mutagenesis (i.e. by insertions, deletions, or substitutions) of one of the aforementioned AAV Caps or its encoding nucleic acid. In some embodiments, the AAV Cap is at least 70%, 75%, 80%, 85%, 90%, 95%, 98%, or 99% or more similar to one or more of the aforementioned AAV Caps.

In some embodiments, the AAV Cap is chimeric, comprising domains from two, three, four, or more of the aforementioned AAV Caps. In some embodiments, the AAV Cap is a mosaic of VP1, VP2, and VP3 monomers originating from two or three different AAV or a recombinant AAV. In some embodiments, a rAAV composition comprises more than one of the aforementioned Caps.

Suitable rep may be derived from any AAV serotype. In one embodiment, the rep is derived from any of the serotypes selected from the group consisting of AAV1, AAV2, AAV3, AAV4, AAV5, AAV6, AAV7, AAV8, or AAV9. In another embodiment, the AAV rep is derived from the serotype AAV2.

Suitable helper genes may be derived from any AAV serotype and include adenovirus E4, E2a and VA.

The AAV rep, AAV cap and genes providing helper functions can be introduced into the cell by incorporating the genes into a vector such as, for example, a plasmid, and introducing the vector into a cell. The genes can be incorporated into the same plasmid or into different plasmids. In one, the AAV rep and cap genes are incorporated into one plasmid and the genes providing helper functions are incorporated into another plasmid.

The AAV vectors of the invention and the polynucleotides comprising AAV rep and cap genes and genes providing helper functions may be introduced into a host cell using any suitable method well known in the art. See Ausubel F, et al, Eds., “Short Protocols in Molecular Biology”, 4th Ed. (John Wiley and Sons, Inc., New York, N.Y., US, 1997), Brown (1995), Watson (1992), Alberts (2008), Innis (1990), Erlich (1989), Sambrook (1989), Bishop (1987), Reznikoff (1987), Davis (1986), and Schleef (2001), supra. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. When the cell lacks the expression of any of the AAV rep and cap genes and genes providing adenoviral helper functions, said genes can be introduced into the cell simultaneously with the AAV vector. Alternatively, the genes can be introduced in the cell before or after the introduction of the AAV vector of the invention.

Methods of culturing packaging cells and exemplary conditions which promote the release of AAV vector particles, such as the producing of a cell lysate, are known in the art. Producer cells are grown for a suitable period of time in order to promote release of viral vectors into the media. Generally, cells may be grown for about 24 hours, about 36 hours, about 48 hours, about 72 hours, about 4 days, about 5 days, about 6 days, about 7 days, about 8 days, about 9 days, up to about 10 days. After about 10 days (or sooner, depending on the culture conditions and the particular producer cell used), the level of production generally decreases significantly. Generally, time of culture is measured from the point of viral production. For example, in the case of AAV, viral production generally begins upon supplying helper virus function in an appropriate producer cell as described herein. Generally, cells are harvested about 48 to about 100, preferably about 48 to about 96, preferably about 72 to about 96, preferably about 68 to about 72 hours after helper virus infection (or after viral production begins).

The AAV vector particles of the invention can be obtained from both: i) the cells transfected with the foregoing and ii) the culture medium of the cells after a period of time post-transfection, preferably 72 hours. Any method for the purification of the AAV vector particles from the cells or the culture medium can be used for obtaining the AAV vector particles of the invention. In a particular embodiment, the AAV vector particles of the invention are purified following an optimized method based on a polyethylene glycol or ammonium sulfate precipitation step and cesium chloride (CsCl) or iodixanol density gradient ultracentrifugation. See Ayuso et al., 2014, Zolotukhin S, et al., Gene Ther. 1999; 6; 973-985. Purified AAV vector particles of the invention can be dialyzed against an appropriate formulation buffer such as PBS, filtered and stored at −80° C. Titers of viral genomes can be determined by quantitative PCR following the protocol described for the AAV2 reference standard material using an appropriate linearized plasmid DNA standard curve. See Aurnhammer C, et al., Hum Gene Ther Methods, 2012, 23, 18-28, D'Costa S, et al., Mol Ther Methods Clin Dev. 2016, 5, 16019.

In some embodiments, the methods further comprise purification steps, such as treatment of the cell lysate with benzonase, purification of the cell lysate with the use of affinity chromatography and/or ion-exchange chromotography. See Halbert C, et al, Methods Mol. Biol. 2004; 246:201-212, Nass S, et al., Mol Ther Methods Clin Dev. 2018 Jun. 15; 9: 33-46.

AAV Rep and Cap proteins and their sequences, as well as methods for isolating or generating, propagating, and purifying such AAV, and in particular, their capsids, suitable for use in producing AAV are known in the art. See Gao, 2004, supra, Russell D, et al, U.S. Pat. No. 6,156,303, Hildinger M, et al, U.S. Pat. No. 7,056,502, Gao G, et al, U.S. Pat. No. 7,198,951, Zolotukhin S, U.S. Pat. No. 7,220,577, Gao G, et al, U.S. Pat. No. 7,235,393, Gao G, et al, U.S. Pat. No. 7,282,199, Wilson J, et al, U.S. Pat. No. 7,319,002, Gao G, et al, U.S. Pat. No. 7,790,449, Gao G, et al, US 20030138772, Gao G, et al, US 20080075740, Hildinger M, et al, WO 2001/083692, Wilson J, et al, WO 2003/014367, Gao G, et al, WO 2003/042397, Gao G, et al, WO 2003/052052, Wilson J, et al, WO 2005/033321, Vandenberghe L, et al, WO 2006/110689, Vandenberghe L, et al, WO 2007/127264, and Vandenberghe L, et al, WO 2008/027084.

### III. Pharmaceutical Compositions of the Invention

In one aspect of the invention, an AAV viral particle of the invention will be in the form of a pharmaceutical composition containing a pharmaceutically acceptable carrier. As used herein “pharmaceutically acceptable carrier” refers to any substantially non-toxic carrier conventionally useable for administration of pharmaceuticals in which the isolated polypeptide of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Suitable pharmaceutically acceptable carriers include any and all solvents, dispersion media, coatings, antibacterial and antifungal agents, isotonic and absorption delaying agents, and the like that are physiologically compatible. Pharmaceutically acceptable carriers also include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the gene therapy vector, use thereof in the pharmaceutical compositions of the invention is contemplated. Supplementary active compounds can also be incorporated into the compositions.

Pharmaceutical compositions of the invention may be formulated for delivery to animals for veterinary purposes (e.g. livestock (cattle, pigs, dogs, mice, rats), and other non-human mammalian subjects, as well as to human subjects.

In a particular embodiment, the pharmaceutical compositions of the present invention are in the form of injectable compositions. The compositions can be prepared as an injectable, either as liquid solutions or suspensions. The preparation may also be emulsified. Suitable excipients are, for example, water, saline, dextrose, glycerol, ethanol, phosphate buffered saline or the like and combinations thereof. In addition, if desired, the preparation may contain minor amounts of auxiliary substances such as wetting or emulsifying agents, pH-buffering agents, adjuvants, surfactant or immunopotentiators.

In a particular embodiment, the AAV particles of the invention are incorporated in a composition suitable for intraocular administration. For example, the compositions may be designed for intravitreal, subretinal, subconjuctival, sub-tenon, periocular, retrobulbar, suprachoroidal, and/or intrascleral administration, for example, by injection, to effectively treat the retinal disorder. Additionally, a sutured or refillable dome can be placed over the administration site to prevent or to reduce “wash out”, leaching and/or diffusion of the active agent in a non-preferred direction.

Relatively high viscosity compositions, as described herein, may be used to provide effective, and preferably substantially long-lasting delivery of the nucleic acid molecules and/or vectors, for example, by injection to the posterior segment of the eye. A viscosity inducing agent can serve to maintain the nucleic acid molecules and/or vectors in a desirable suspension form, thereby preventing deposition of the composition in the bottom surface of the eye. Such compositions can be prepared as described in U.S. Pat. No. 5,292,724, the entire contents of which are hereby incorporated herein by reference.

Sterile injectable solutions can be prepared by incorporating the compositions of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation include vacuum drying and freeze-drying which yields a powder of the active ingredient plus any additional desired ingredient from a previously sterile-filtered solution thereof.

Toxicity and therapeutic efficacy of nucleic acid molecules described herein can be determined by standard pharmaceutical procedures in cell cultures or experimental animals, e.g., for determining the ED₅₀ (the dose therapeutically effective in 50% of the population). Data obtained from cell culture assays and/or animal studies can be used in formulating a range of dosage for use in humans. The dosage typically will lie within a range of concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays.

### IV. Methods of the Invention

The present invention also provides methods of use of the compositions of the invention, which generally include contacting an ocular cell with an AAV viral particle or pharmaceutical composition comprising an AAV particle of the invention.

Accordingly, in one aspect, the present invention provides methods for prolonging the viability of a photoreceptor cell, e.g., a photoreceptor cell, compromised by degenerative ocular disorder, e.g., retinitis pigmentosa, age related macular degeneration, cone rod dystrophy, and rod cone dystrophy. The methods generally include contacting the cell with an AAV viral particle or pharmaceutical composition comprising an AAV particle of the invention.

The present invention further provides methods for treating a degenerative ocular disorder in a subject having a degenerative ocular disorder, e.g., retinitis pigmentosa, age related macular degeneration, cone rod dystrophy, and rod cone dystrophy. The methods include administering to the subject a therapeutically effective amount of an AAV viral particle or pharmaceutical composition comprising an AAV particle of the invention.

The present invention also provides methods for preventing a degenerative ocular disorder in a subject having a degenerative ocular disorder, e.g., retinitis pigmentosa, age related macular degeneration, cone rod dystrophy, and rod cone dystrophy. The methods include administering to the subject a prohylatically effective amount of an AAV viral particle or pharmaceutical composition comprising an AAV particle of the invention.

In another aspect, the present invention provides methods of treating a subject having retinitis pigmentosa. The methods include administering to the subject a therapeutically effective amount of an AAV viral particle or pharmaceutical composition comprising an AAV particle of the invention.

In another aspect, the present invention provides methods of treating a subject having age-related macular degeneration. The methods include administering to the subject a therapeutically effective amount of an AAV viral particle or pharmaceutical composition comprising an AAV particle of the invention.

Generally, methods are known in the art for viral infection of the cells of interest. The virus can be placed in contact with the cell of interest or alternatively, can be injected into a subject suffering from a disorder associated with photoreceptor cell oxidative stress.

Guidance in the introduction of the compositions of the invention into subjects for therapeutic purposes are known in the art and may be obtained in the above-referenced publications, as well as in U.S. Pat. Nos. 5,631,236, 5,688,773, 5,691,177, 5,670,488, 5,529,774, 5,601,818, and PCT Publication No. WO 95/06486, the entire contents of which are incorporated herein by reference.

The compositions of the invention may be delivered to a subject by, for example, intravenous injection, local administration (see, e.g., U.S. Pat. No. 5,328,470), stereotactic injection (see, e.g., Chen et al. (1994) Proc. Natl. Acad. Sci. U.S.A. 91:3054-3057), or by in vivo electroporation (see, e.g., Matsuda and Cepko (2007) Proc. Natl. Acad. Sci. U.S.A. 104:1027-1032). Preferably, the compositions of the invention are administered to the subject locally. Local administration of the compositions described herein can be by any suitable method in the art including, for example, injection (e.g., intravitreal or subretinal, subvitreal, subconjuctival, sub-tenon, periocular, retrobulbar, suprachoroidal, and/or intrascleral injection), gene gun, by topical application of the composition in a gel, oil, or cream, by electroporation, using lipid-based transfection reagents, transcleral delivery, by implantation of scleral plugs or a drug delivery device, or by any other suitable transfection method.

Application of the methods of the invention for the treatment and/or prevention of a disorder can result in curing the disorder, decreasing at least one symptom associated with the disorder, either in the long term or short term or simply a transient beneficial effect to the subject.

Accordingly, as used herein, the terms “treat,” “treatment” and “treating” include the application or administration of compositions, as described herein, to a subject who is suffering from a degenerative ocular disease or disorder, or who is susceptible to such conditions with the purpose of curing, healing, alleviating, relieving, altering, remedying, ameliorating, improving or affecting such conditions or at least one symptom of such conditions. As used herein, the condition is also “treated” if recurrence of the condition is reduced, slowed, delayed or prevented.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Therapeutically effective amount,” as used herein, is intended to include the amount of a composition of the invention that, when administered to a patient for treating a degenerative ocular disease, is sufficient to effect treatment of the disease (e.g., by diminishing, ameliorating or maintaining the existing disease or one or more symptoms of disease). The “therapeutically effective amount” may vary depending on the composition, how the composition is administered, the disease and its severity and the history, age, weight, family history, genetic makeup, stage of pathological processes mediated by the disease expression, the types of preceding or concomitant treatments, if any, and other individual characteristics of the patient to be treated.

“Prophylactically effective amount,” as used herein, is intended to include the amount of a composition that, when administered to a subject who does not yet experience or display symptoms of e.g., a degenerative ocular disorder, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the composition, how the composition is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of a composition that produces some desired local or systemic effect at a reasonable benefit/risk ratio applicable to any treatment. A composition employed in the methods of the present invention may be administered in a sufficient amount to produce a reasonable benefit/risk ratio applicable to such treatment.

Subjects suitable for treatment using the regimens of the present invention should have or are susceptible to developing a degenerative ocular disease or disorder. For example, subjects may be genetically predisposed to development of the disorders. Alternatively, abnormal progression of the following factors including, but not limited to visual acuity, the rate of death of cone and/or rod cells, night vision, peripheral vision, attenuation of the retinal vessels, and other ophthalmoscopic factors associated with degenerative ocular disorders such as retinitis pigmentosa may indicate the existence of or a predisposition to a retinal disorder.

In one embodiment, the disorder includes, but not limited to, retinitis pigmentosa, age related macular degeneration, cone rod dystrophy, and rod cone dystrophy. In other embodiments, the disorder is not associated with blood vessel leakage and/or growth. In certain embodiments, the disorder is not associated with diabetes. In another embodiment, the disorder is not diabetic retinopathy. In further embodiments, the disorder is not NARP (neuropathy, ataxia and retinitis pigmentosa). In one embodiment, the disorder is a disorder associated with decreased viability of cone and/or rod cells. In yet another embodiment, the disorder is a genetic disorder.

The compositions, as described herein, may be administered as necessary to achieve the desired effect and depend on a variety of factors including, but not limited to, the severity of the condition, age and history of the subject and the nature of the composition, for example, the identity of the genes or the affected biochemical pathway.

The pharmaceutical compositions of the invention may be administered in a single dose or, in particular embodiments of the invention, multiples doses (e.g. two, three, four, or more administrations) may be employed to achieve a therapeutic effect.

The therapeutic or preventative regimens may cover a period of at least about 2, 3, 4, 5, 6, 7, 8, 9, 10, 11, 12, 13, 14, 15, 16, 17, 18, 19, 20, 21, 22, 23 or 24 weeks, or be chronically administered to the subject.

In one embodiment, the viability or survival of photoreceptor cells, such as cones cells, is, e.g., about 1 week, about 2 weeks, about 3 weeks, about 4 weeks, about 5 weeks, about 6 weeks, about 7 weeks, about 8 weeks, about 3 years, about 4 years, about 5 years, about 10 years, about 15, years, about 20 years, about 25 years, about 30 years, about 40 years, about 50 years, about 60 years, about 70 years, and about 80 years.

In general, the nucleic acid molecules and/or the vectors of the invention are provided in a therapeutically effective amount to elicit the desired effect, e.g., increase Nrf2 expression. The quantity of the viral particle to be administered, both according to number of treatments and amount, will also depend on factors such as the clinical status, age, previous treatments, the general health and/or age of the subject, other diseases present, and the severity of the disorder. Precise amounts of active ingredient required to be administered depend on the judgment of the gene therapist and will be particular to each individual patient. Moreover, treatment of a subject with a therapeutically effective amount of the nucleic acid molecules and/or the vectors of the invention can include a single treatment or, preferably, can include a series of treatments. It will also be appreciated that the effective dosage used for treatment may increase or decrease over the course of a particular treatment. Changes in dosage may result from the results of diagnostic assays as described herein. The pharmaceutical compositions can be included in a container, pack, or dispenser together with instructions for administration.

In some embodiments, a therapeutically effective amount or a prophylactically effective amount of a viral particle of the invention (or pharmaceutical composition of the invention) is in titers ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹² viral particles (vp).

In some embodiments, a therapeutically effective amount or a prophylactically effective amount of a viral particle of the invention (or pharmaceutical composition of the invention) is in genome copies (“GC”), also referred to as “viral genomes” (“vg”) ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹² vg.

Any method known in the art can be used to determine the genome copy (GC) number of the viral compositions of the invention. One method for performing AAV GC number titration is as follows: purified AAV viral particle samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In various embodiments, the methods of the present invention further comprise monitoring the effectiveness of treatment. For example, visual acuity, the rate of death of cone and/or rod cells, night vision, peripheral vision, attenuation of the retinal vessels, and other ophthalmoscopic changes associated with retinal disorders such as retinitis pigmentosa may be monitored to assess the effectiveness of treatment. Additionally, the rate of death of cells associated with the particular disorder that is the subject of treatment and/or prevention, may be monitored. Alternatively, the viability of such cells may be monitored, for example, as measured by phospholipid production. The assays described in the Examples section below may also be used to monitor the effectiveness of treatment (e.g., electroretinography—ERG).

In certain embodiments of the invention, a composition of the invention is administered in combination with an additional therapeutic agent or treatment. The compositions and an additional therapeutic agent can be administered in combination in the same composition or the additional therapeutic agent can be administered as part of a separate composition or by another method described herein.

Examples of additional therapeutic agents suitable for use in the methods of the invention include those agents known to treat retinal disorders, such as retinitis pigmentosa and age-related macular degeneration and include, for example, fat soluble vitamins (e.g., vitamin A, vitamin E, and ascorbic acid), calcium channel blockers (e.g., diltiazem) carbonic anhydrase inhibitors (e.g., acetazolamide and methazolamide), anti-angiogenics (e.g., antiVEGF antibodies), growth factors (e.g., rod-derived cone viability factor (RdCVF), BDNF, CNTF, bFGF, and PEDF), antioxidants, other gene therapy agents (e.g., optogenetic gene threrapy, e.g., channelrhodopsin, melanopsin, and halorhodopsin), and compounds that drive photoreceptor regeneration by, e.g., reprogramming Müller cells into photoreceptor progenitors (e.g., alpha-aminoadipate). Exemplary treatments for use in combination with the treatment methods of the present invention include, for example, retinal and/or retinal pigmented epithelium transplantation, stem cell therapies, retinal prostheses, laser photocoagulation, photodynamic therapy, low vision aid implantation, submacular surgery, and retinal translocation.

This invention is further illustrated by the following examples which should not be construed as limiting. The contents of all references, patents and published patent applications cited throughout this application, as well as the Figures, are hereby incorporated by reference.

### EXAMPLES

### Example 1: AAV Genome Structure is Correlated with Ocular Toxicity

Adeno-associated viruses (AAVs) are small single-stranded DNA viruses in the Parvoviridae family that have several advantages as somatic gene therapy vectors. Recombinant AAV genomes typically lack viral genes and do not efficiently integrate into the host genome, eliminating the risk of insertional mutagenesis. They establish as stable episomes and express transgenes indefinitely in post-mitotic cells. Naturally existing AAV variants, together with an array of engineered variants, can infect a large variety of tissues and cell types in both animals and humans (Dalkara et al., 2013; Gao et al., 2004; Zinn et al., 2015). These capsid variants can enable more targeted infection of a selected set of cell types, with transgene expression further specified through the use of transcription regulatory sequences. Finally, AAV is non-pathogenic, even in its wild type form, which has predicted its safety as a gene therapy vector. Multiple clinical trials have indeed born this out (Bainbridge et al., 2008; Dixon et al., 2011; Hauswirth et al., 2008; Maguire et al., 2008).

AAV has emerged as the vector of choice for retinal diseases. There are many recessive disease genes, and complementation by a vector-encoded gene can lead to an improvement in vision (Acland et al., 2001). The target cells for retinal gene therapy are most often the photoreceptors and retinal pigment epithelial (RPE) cells, as most genetic retinal diseases initiate with dysfunction, often followed by death, of these cell types. There are two types of photoreceptors: rods, necessary for dim light vision, and cones, required for bright light and color vision. Vision initiates with the detection of light in an elaborate and specialized photoreceptor structure, the outer segment (OS), whose morphology can serve as an indicator of photoreceptor health. Photoreceptor cells are supported by the RPE, an epithelial layer with processes in close contact with the photoreceptor OS's. Injections into the subretinal space, the virtual space between the RPE and photoreceptors, is thus the injection site for most ocular human gene therapy. In addition to these target cell types being accessible for gene therapy, the eye offers several other advantages for somatic gene therapy. It is relatively immune privileged, anatomically compartmentalized, and can be targeted by established clinical interventions. Its target cells do not replicate and thus do not need integrating viruses. One further attribute that is particularly valuable, given the expense of generating pure viral vectors, is that only a small amount of virus is needed for local administration. These advantages stand in contrast to the systemic administration required for large organs, such as liver or muscle, and led to the approval of AAV encoding the RPE65 gene (Luxturna) for Leber's Congenital Amaurosis 2 (LCA2), a rare retinal disease (Bainbridge et al., 2008; Hauswirth et al., 2008; Maguire et al., 2008). AAV has proven to be safe in the LCA2 clinical trials, as well as in several clinical trials for other ocular diseases such as choroideremia and retinitis pigmentosa (Ghazi et al., 2016; MacLaren et al., 2014).

Despite the safety of the human trials to date, the expansion of AAV therapy to a larger number of patients may reveal some underlying problems. In addition, it likely will be desirable to increase the dose in future trials, which may similarly lead to problems. Current subretinal injections lead to infection only of cells near the injection site, which comprise a small percentage of target cells. A more complete infection requiring a larger viral load likely will offer greater improvement to vision. However, toxicity associated with higher doses has been seen in animals, including non-human primates (NHPs), in both non-ocular and ocular tissues. An early indication came from treatment of hemophilia B by AAV-mediated Factor IX expression. AAV infection in the liver triggered memory T cells reactive with the capsid, which cleared the infected cells and resulted in only transient expression of Factor IX (Mingozzi and High, 2013). More recently, systemic delivery of high doses of an AAV variant to NHP's and pigs led to neurotoxicity, due to an uncharacterized mechanism (Hinderer et al., 2018). Specifically in the eye, AAV toxicity was observed using AAV2-CNGA3 to treat color blindness (achromatopsia) in sheep, in which two animals had loss of photoreceptors and RPE, and one animal injected with high dose AAV showed both retinal atrophy and lymphocytic infiltration (Gootwine et al., 2017). In one of the LCA gene therapy trials, strong evidence of an inflammatory response emerged. Five out of 8 subjects injected with the higher dose of AAV2-RPE65 (1E12 vector genomes (vg)/eye) developed various degrees of intraocular inflammation (Bainbridge et al., 2015).

AAV gene therapy for retinal degeneration has been developed, hoping to create vectors that are able to prolong photoreceptor survival and function independent of the disease gene. We found that subretinal delivery of some, but not all, AAVs consistently induced cone OS shortening, reduction of the outer nuclear layer (ONL) where rods and cones reside, and dysmorphic RPE, in mice. We began tracking many aspects of the preparations and vector structures and did not find that the ocular toxicity tracked with preparation methods, virus core facilities that made the preparation, endotoxin level, or cellular protein contaminants. To search for the source of the toxicity, we tested virus stocks with different cis-regulatory sequences, transgenes, and capsids. We found a strong correlation between AAV structure and toxicity. AAVs incorporating all broadly active promoters tested, including cytomegalovirus immediate-early promoter (CMV), human ubiquitin C promoter (UbiC), and chicken beta actin promoter (CAG), as well an RPE-specific promoter, the human Best1 promoter, were found to be toxic. In contrast, vectors with photoreceptor-specific promoters tested, including human Red Opsin (Busskamp et al., 2010; Wang et al., 1992), human Rhodopsin (Allocca et al., 2007; Busskamp et al., 2010), and human Rhodopsin kinase (Khani et al., 2007), were not toxic. As might be expected, toxicity among the toxic group of vectors was associated with the dose, while administration of the highest dose possible of the photoreceptor-specific vectors did not lead to toxicity. The RPE was more sensitive to virus toxicity than photoreceptors. Microglia activation and inflammatory cytokine expression were triggered by the toxic viruses. These data highlight the need to develop sensitive assays of viral toxicity for the organ and cell types that are being targeted. Such assays enable the design of vectors that can be used to safely deliver higher doses of vectors, potentially leading to both greater safety and efficacy.

The following Materials and Methods were used in this Example.

### Mice

CD1 and C57BL/6 mice were purchased from Charles River Laboratories and were kept on a 12-hour light/12-hour dark cycle. All animal studies were approved by the Institutional Animal Care and Use Committee at Harvard University.

### Plasmids

pAAV-CMV-GFP, pAAV-CMV-null, pAAV-UbiC-GFP vector plasmids were obtained from Harvard DF/HCC DNA Resource Core (pAAV-GFP and pAAV-MCS8 deposited by Dr. Jeng-Shin Lee to Harvard DF/HCC DNA Resource Core). pAAV-CAG-GFP (from E. Boyden Lab) was obtained from Addgene (Addgene #37825).

pAAV-Red O-GFP and pAAV-Rho-GFP were kind gifts of B. Roska lab (Busskamp et al., 2010). pAAV-hRK-ZsGreen was a kind gift from T. Li lab at NEI (Khani et al., 2007). pAAV-hBest1-GFP was cloned by replacing the CMV promoter of the pAAV-CMV-SV40-GFP-BGHpA vector (Upenn virus core, ID: PV0101) with the −585/+38 bp region of human Best1 promoter region by Gibson ligation (Esumi et al., 2004).

pAAV-CMV-TdTomato and pAAV-hBest1-mApple were cloned by replacing GFP with TdTomato and mApple coding sequence by Gibson ligation. pAAV rep/Cap 2/2, 2/8 and Adenovirus helper plasmids were obtained from University of Pennsylvania Vector Core (Philadelphia, Pa., USA). pAAV2 7m8 and pAAV Anc80 plasmids were kind gifts from Dr. J. Flannery (Dalkara et al., 2013) and Dr. L. Vandenberghe (Zinn et al., 2015).

### AAV Vector Production and Titering

Recombinant AAV8 and AAV2 vectors were produced as previously described (Grieger et al., 2006). Briefly, AAV vector, Rep/Cap packaging plasmid, and adenoviral helper plasmid were mixed with polyethylenimine and added to HEK293T cells (catalog HCL4517; Thermo Scientific). 72 hours after transfection, supernatant was collected for AAV8 preparations, and cells were harvested for AAV2 preparations (Vandenberghe et al., 2010). AAV8 viruses in the supernatant were precipitated (mixed with 8.5% w/v PEG-6000 and 0.4 M NaCl for 2 hours at 4° C.), centrifuged at 7,000 g for 10 minutes, and resuspended in virus buffer (150 mM NaCl and 20 mM Tris, pH 8.0). For AAV2 viruses, the cell pellet was resuspended in virus buffer, followed by 3 cycles of freeze-thawing, and Dounce homogenized. Cell debris was pelleted at 5,000 g for 20 minutes, and the supernatant was run on an iodixanol gradient. Recovered AAV vectors were washed 3 times with PBS using Amicon 100K columns (EMD Millipore). RT-PCRs and protein gels were run to determine virus titers.

### AAV Injection

Subretinal injection into P0 neonate eyes was performed as previously described (Matsuda and Cepko, 2004; Wang et al., 2014). Approximately 0.3 μl AAV was introduced into the subretinal space using a pulled angled glass pipette controlled by a FemtoJet (Eppendorf). The fellow eyes were uninjected for within-animal controls.

### Optomotor Responses

The optomotor responses of mice were measured using the OptoMotry System (CerebralMechanics) with minor modifications, as previously described (Xiong et al., 2015; Xue et al., 2015a). Only the photopic vision was tested, at a background light of ˜70 cd/m² in this study. An examiner tested the mouse visual acuity (i.e. maximal spatial frequency) and the contrast sensitivity (i.e. minimal contrast) separately and blindly (i.e. without knowing which AAVs were injected in which eyes) with the aid of a computer program. In the acuity test, the contrast of the grates was set at 100%. In the contrast sensitivity test, the spatial frequency was set as 0.128 cycles/degree. In both of the tests, the temporal frequency was set at 1.5 Hz. During each test, a computer program determined the moving direction of the grates (i.e. clockwise or counter-clockwise) and the parameters at each testing episode. The examiner could see the moving direction of the grates through virtual radiances on the screen but could not see the parameters, in order to minimize human bias. In each testing episode (−5 seconds), the examiner reported “yes” (or “no”) to the system if observation of the mouse provided (or not) an optomotor response that matched the grating movement. After a series of test episodes, the same computer program determined the acuity or contrast sensitivity of the right eyes (i.e. counter-clockwise) and the left eye (i.e. clockwise). The acuity was recorded as it was, while the contrast sensitivity was recorded as its reverse for analysis.

### Optical Coherence Tomography (OCT)

OCT images of mouse eyes were taken by a commercially available OCT2 system in combination with the MicronlV fundus imaging system (Phoenix Research Labs). The animals were anesthetized with a ketamine/xylazine (100/10 mg/kg) cocktail. The eyes were treated with a drop of 5% phenylephrine and 0.5% tropicamide solution to dilate the pupils, and a drop of GONAK 2.5% hypromellose solution (Akorn) to keep the lens hydrated. Fundus images were taken with a filter set of Exciter (FF01-469/35-25, Semrock) and Barrier (FF03-525/50, Semrock) that were selected for spectra to visualize GFP. The OCT image of the retina was taken near the optic nerve head, and the imaging location was marked on the fundus image by a long green arrow.

### Electoretinography (ERG)

ERGs were performed in vivo with the Espion E3 System (Diagonsys LLC) as previously described (Xiong et al., 2015; Xue et al., 2015a). A new ERG protocol was created to characterize the rod and cone responses with a minimal number of flash steps based on our previous studies of wild type mice (Xue et al., 2015b, 2017). In brief, mice were dark-adapted overnight in a cabinet, anesthetized, and pupils were dilated as described above for the OCT imaging, and placed on a heating pad throughout the tests. Goldwire electrodes were then applied to the surface of the cornea with a drop of PBS as the immersion medium. The reference electrode and ground electrode were applied sub-cutaneously near the tail and under the scalp, respectively. The above steps were all done under a dim red headlight, and the mouse was stabilized in complete darkness for 3 minutes before the scotopic test. The scotopic response was elicited, recorded, and averaged at multiple 0.1 cd s/m² 530 nm flashes. Photopic vision was probed under 30 cd/m² background light after 12 minutes light adaptation with multiple flashes at 1 (peak), 10 (peak), 100 (Xenon), 1000 (Xenon) cd s/m². The amplitude and implicit time of a-wave and b-wave were measured after the recording accordingly.

### Flow Cytometry

Retinas were dissected from adult CX3CR1^{gfp/gfp} (Jackson Laboratory, Bar Harbor, Me.), in which microglia are labeled with GFP (Jung et al., 2000). Individual retinas were isolated and treated with activated papain (Worthington Biochemicals, Lakewood, N.J.) for five minutes at 37° C., followed by manual trituration to dissociate cells. Cells were subsequently washed twice with 2% fetal bovine serum and 2 mM EDTA in PBS, incubated with DAPI at a concentration of 0.5 μg/mL for five minutes at 4° C., and passed through a 40 p.m filter prior to analysis. Flow cytometry was conducted on a BD/Cytek FACSCalibur DxP11 running FACSDiva software (Becton Dickinson, San Jose, Calif.). Over 10,000 live events were recorded for each sample. Data analysis was performed using FlowJo 10 (Tree Star, Ashland, Oreg.) excluding doublet and dead cell (DAPI+) events. Microglia were quantified as the percentage of live singlet events that were GFP+.

### Immunohistochemistry of Whole Eye Mounts

After sacrifice with CO₂, eyes were rapidly enucleated, dissected from tendons and extraocular muscles, and fixed in 4% paraformaldehyde for 2 hours at room temperature. The anterior segment, lens, and vitreous were then removed. The posterior segment eye cups were blocked with 4% heat-inactivated goat serum and 1% triton in PBS for one hour at room temperature. Eye cups were then incubated in primary antibody (rabbit anti-cone arrestin, EMD Millipore AB15282, Burlington, Mass.) diluted 1:100 in the blocking buffer for two days, rinsed 3 times in PBS for 30 minutes each, and stained with secondary antibody solution containing donkey anti-rabbit Alexa Fluor 647 (Jackson ImmunoResearch, West Grove, Pa.) at 1:100 and phalloidin conjugated to Alexa Fluor 568 at 1:100 (Thermofisher, Waltham, Mass.) for 2 days. Eye cups were rinsed 3 times again in PBS for 30 minutes each. Radial cuts were made to enable flat-mounting of the eyes on coverslips. The whole eye mounts were then imaged on a Nikon Ti W1 Yokogawa Spinning Disk Microscope using a 20× objective.

### Retinal Section and Histology

Eyes were enucleated, and retinae were dissected and fixed in 4% formaldehyde for 30 minutes at room temperature. Fixed retinae were cryoprotected in 5%, 15%, and 30% sucrose in PBS for a few hours and embedded in OCT on dry ice. Sections (20 p.m thick) were cut on a cryostat (Leica). Retinal sections or whole retinal cups were blocked in 5% bovine serum albumin in PBST (PBS with 0.1% Triton X-100), stained with primary antibodies at 4° C. overnight, and washed 3 times with PBST. Primary antibodies used in this study included: rabbit anti—red/green opsin (1:300; AB5405; EMD Millipore); goat anti—blue opsin (1:100; sc-14365; Santa Cruz Bio-technology Inc.); rabbit anti-GFAP (1:500; Z0344; DAKO); rabbit anti Iba-1 (1:1000, PAS-21274, Thermofisher) and rhodamine-conjugated and FITC-conjugated PNA (1:1,000; Vector Laboratories). Sections were stained using secondary antibodies, including donkey anti—rabbit CY3, donkey anti—rabbit Alexa Fluor 647, and donkey anti—goat Alexa Fluor 647 (all used at 1:1,000; Jackson ImmunoResearch), and were co-stained with DAPI in the dark for 2 hours at room temperature and mounted in Fluoromount-G (SouthernBiotech). Images were taken using a 40× objective with Z-stacks on a Zeiss LSM780 confocal microscope. Images used for comparison between groups were taken side by side at the same confocal settings.

### Statistics

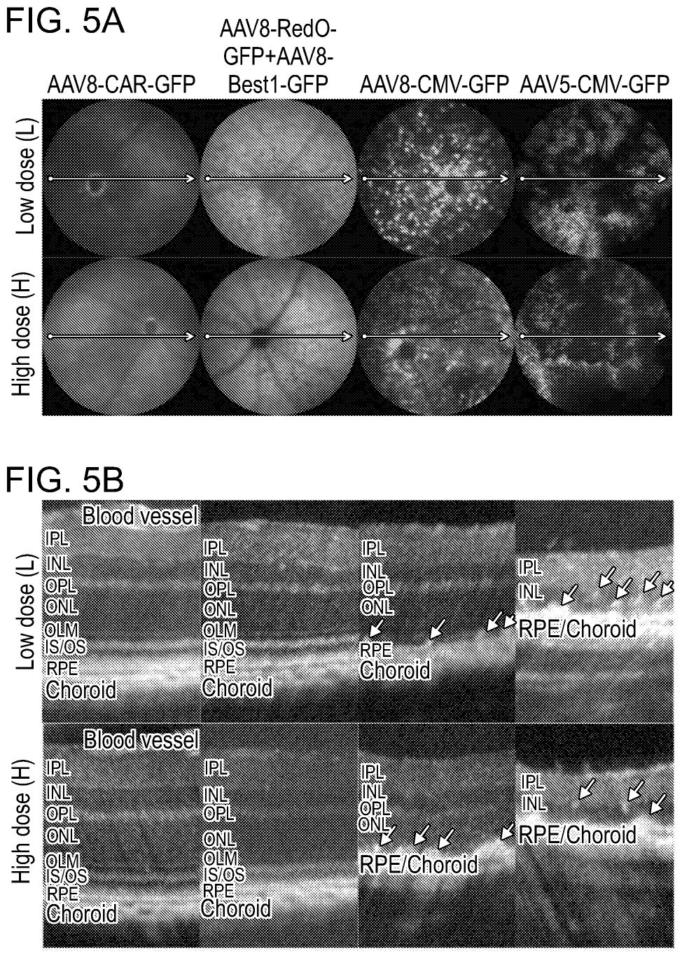

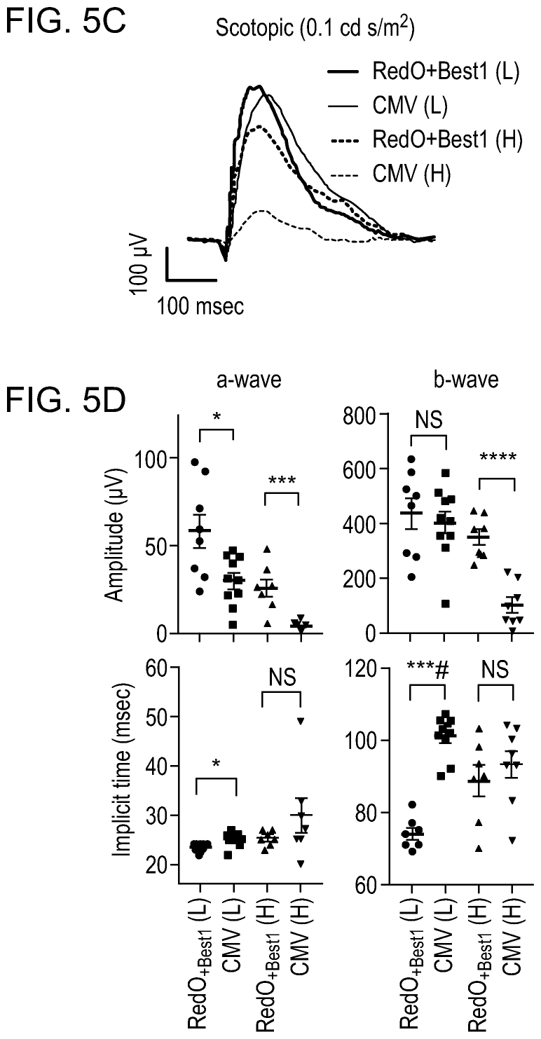

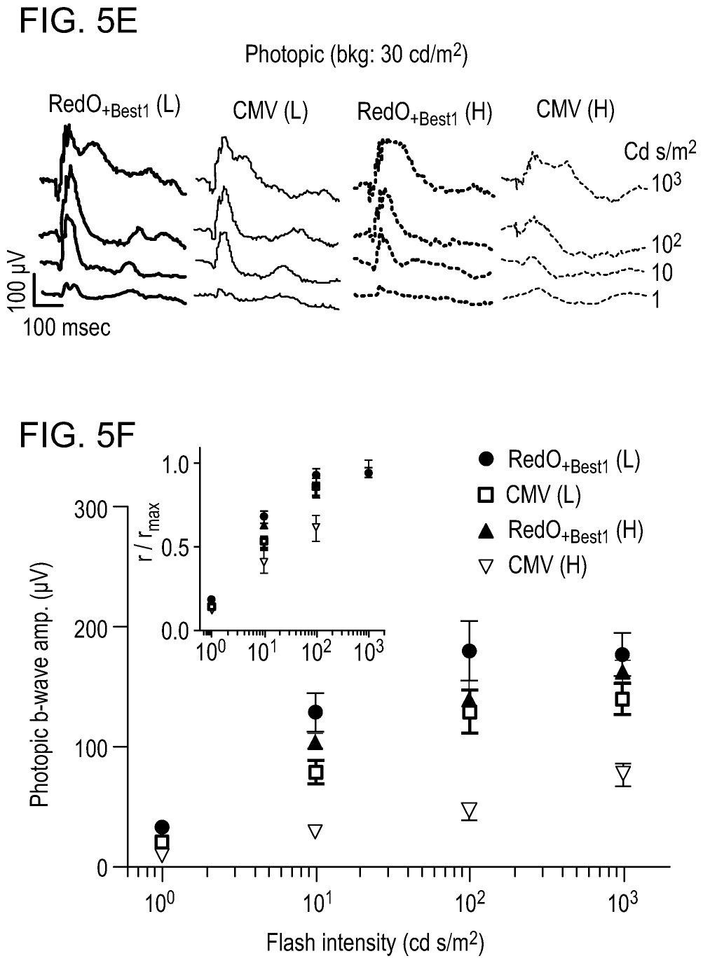

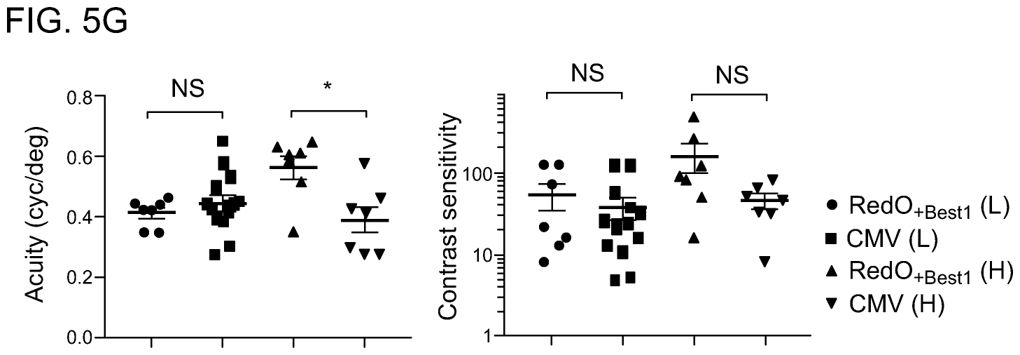

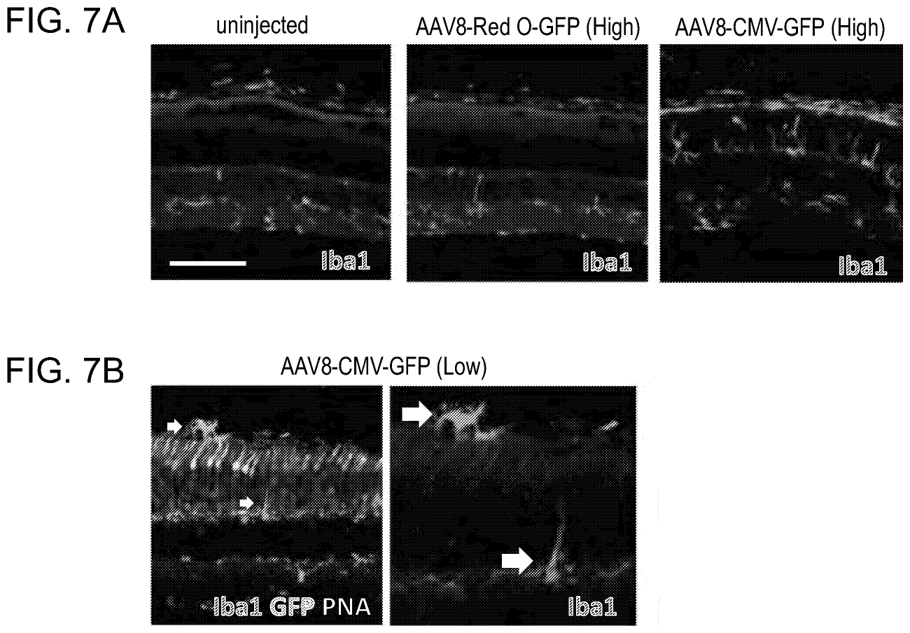

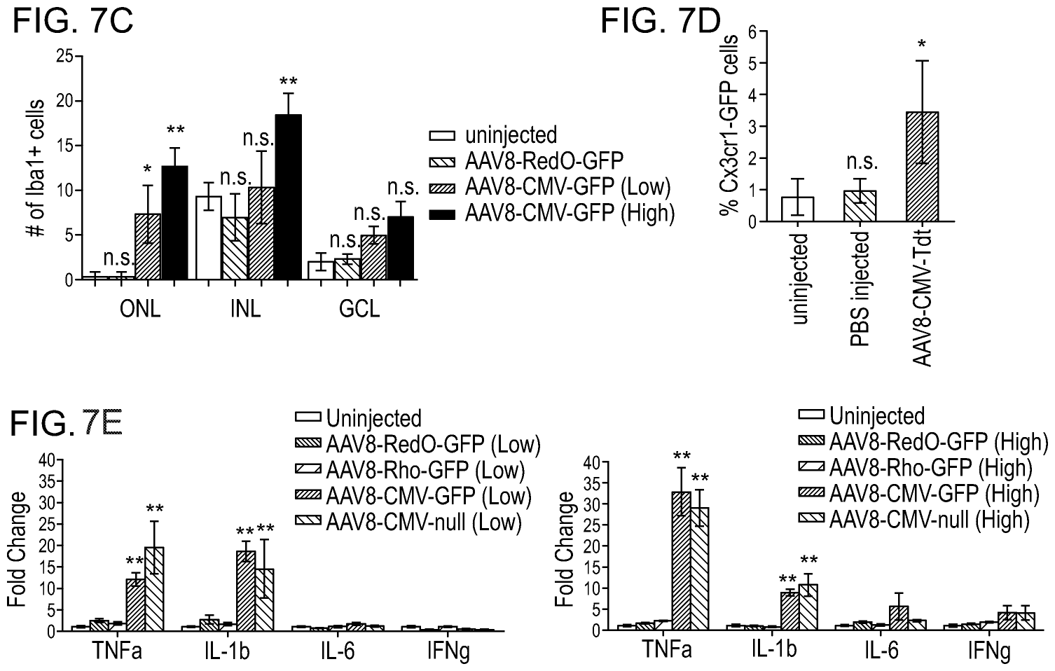

Data were represented as Mean±SD in FIGS. 1C, 2B and 7C-D and as Mean±SEM in FIGS. 5 and 7E. Sample sizes were indicated for each experiment. One-way ANOVA analysis with Tukey test was performed to compare multiple groups, and unpaired student's t-test was performed to compare two groups. GraphPad Prism was used to perform statistical analysis and make figures.

Photoreceptor Toxicity is Induced by AAV Vectors with a Broadly Active or an RPE Promoter, but not Photoreceptor-Specific Promoters.

Serotype 8 AAV (AAV8) viruses expressing either GFP or no transgene (“null”) under the control of different promoters were injected subretinally into neonatal wild type mice (CD-1). The retinas and RPE were harvested for histology at 30 days post infection (or as indicated). The cytomegalovirus (CMV) promoter/enhancer sequence plus a human beta-globin intron II drives robust transgene expression in cone photoreceptors, as well as other cell types (Xiong et al., 2015). AAV8-CMV-GFP induced retinal toxicity, indicated by cone OS shortening, ONL thinning, cone photoreceptor loss, and upregulation of GFAP in a resident glial cell type, the Muller glia (FIGS. 1A-C). In contrast, GFP expression driven by the photoreceptor-specific promoters, including a human red opsin (RedO) promoter (Busskamp et al., 2010; Wang et al., 1992), a human rhodopsin (RHO) promoter (Allocca et al., 2007; Busskamp et al., 2010), or a human rhodopsin kinase (hRK) promoter (Khani et al., 2007), induced no retinal toxicity (FIGS. 1A-C and data not shown). Preliminary studies using the mouse cone arrestin promoter similarly showed no toxicity (data not shown) (Busskamp et al., 2010; Zhu et al., 2002). In order to determine if toxicity was due to protein expression, a vector, AAV-CMV-null was created by deletion of the GFP gene from this vector. AAV8-CMV-null was just as toxic as AAV8-CMV-GFP.

RPE Toxicity is Induced by AAV Vectors with Broadly Active Promoters and a RPE-Specific Promoter.

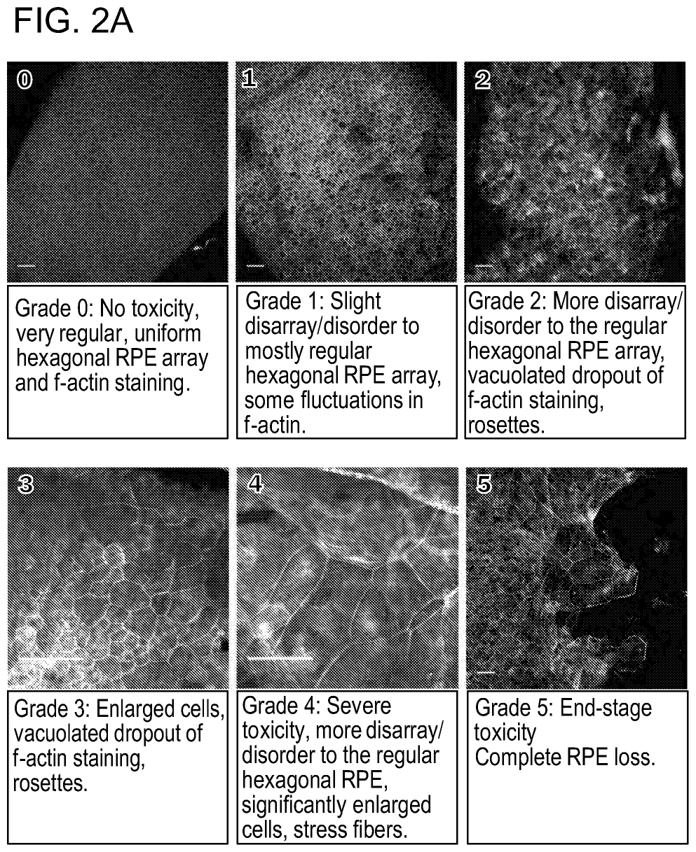

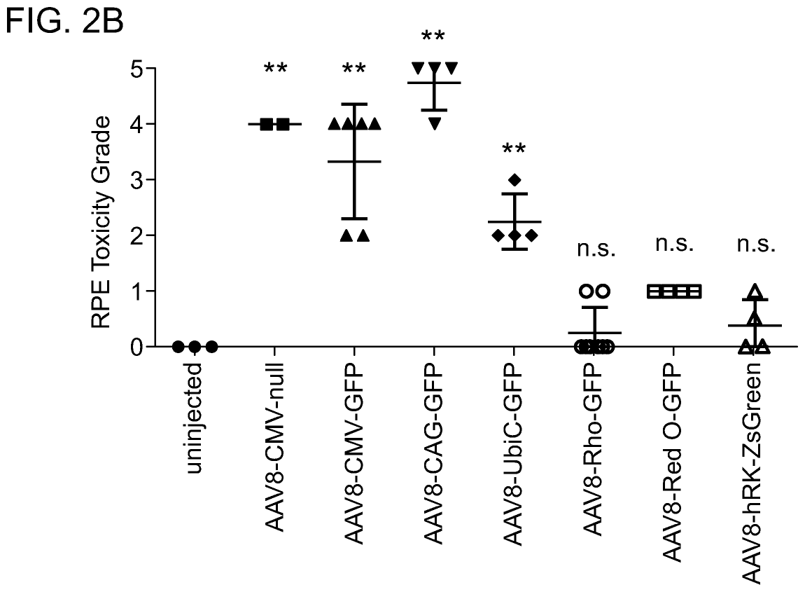

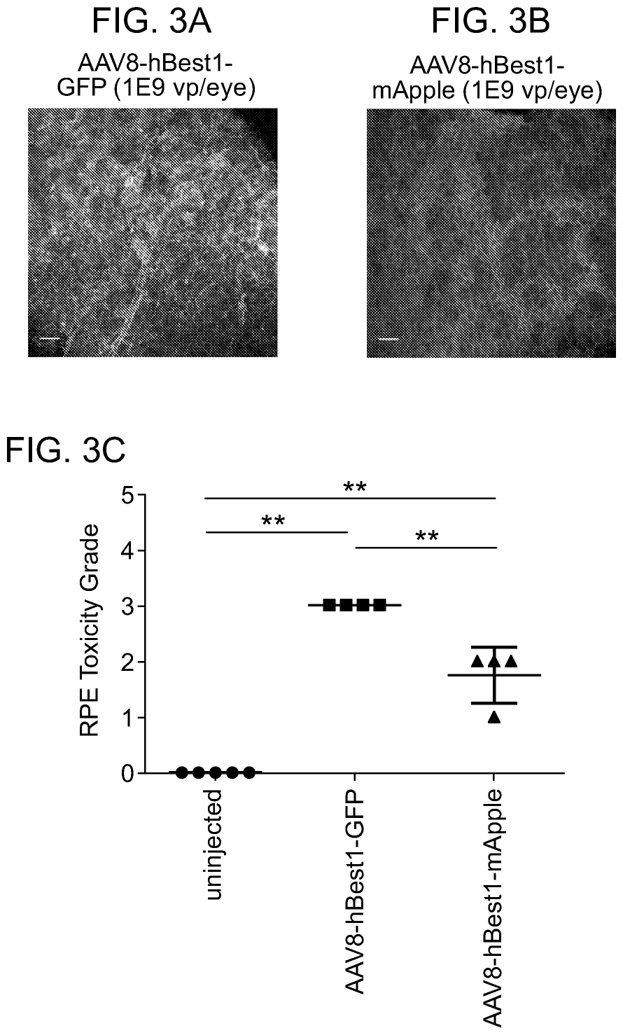

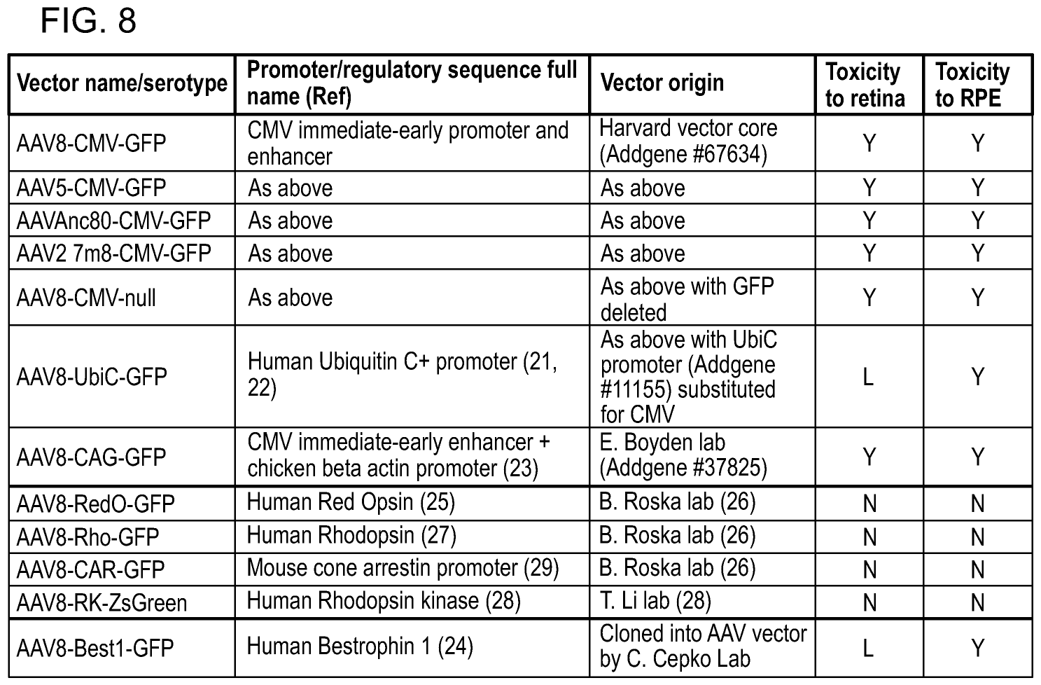

Promoter-specific AAV toxicity also was observed in the RPE, which is efficiently transduced by subretinally delivered AAV viruses. A semi-quantitative assay to measure the RPE toxicity level was developed in order to compare among vectors. Whole RPE flatmounts were stained with phalloidin, which outlines the hexagonal RPE array, imaged with a spinning disc microscope, and scored for the morphology. The RPE flatmounts were assigned six grades, with grade 0 being completely normal and grade 5 being complete RPE loss (FIG. 2A). Four representative areas in the mid-periphery of each flatmount were imaged and then evaluated by four independent scorers who did not know the vector type/dose, with the average score shown in Table 1. With this evaluation system, it was found that the broadly active promoters CMV, CAG, and UbiC induced strong RPE toxicity, while none of the photoreceptor-specific promoters induced RPE toxicity (FIG. 2B). We also included the human Best1 (hBest1) promoter, which drives strong transgene expression in the RPE at a level comparable to that of CMV or CAG (Esumi et al., 2004). Interestingly, AAV8 with hBest1 promoter also induced RPE toxicity in the RPE (FIGS. 3A-C). FIG. 8 shows a strong correlation between toxicity and the promoter used.

### Relationship of AAV Toxicity to Dose and Capsid Type.

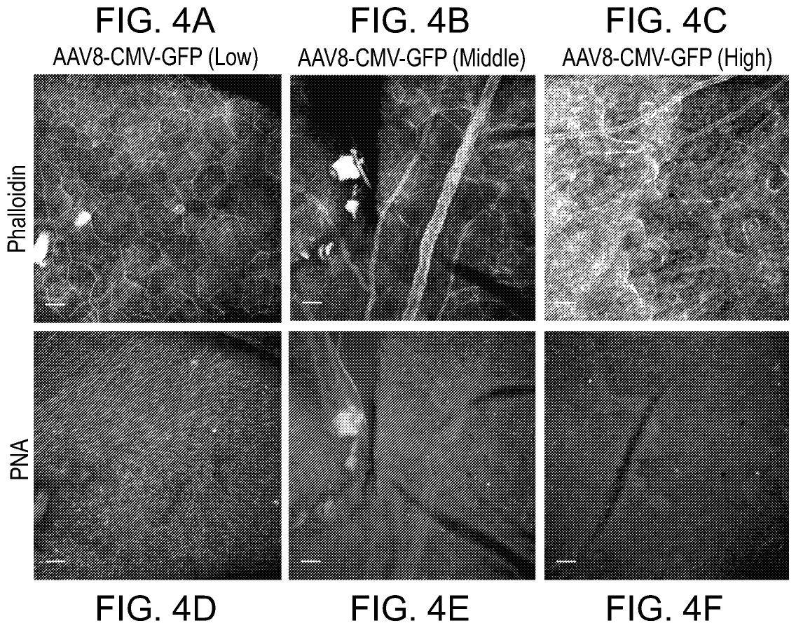

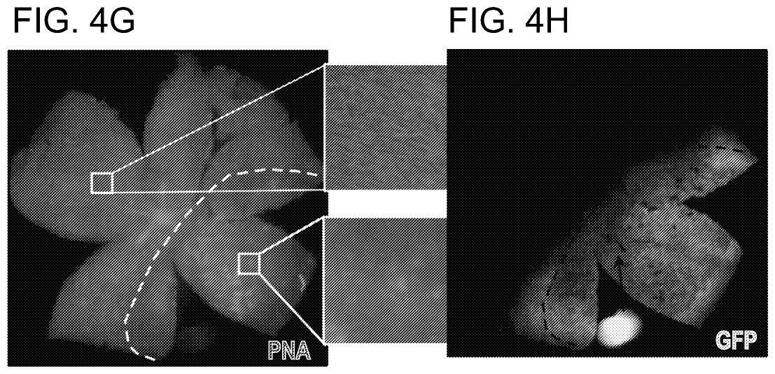

AAV vectors are injected subretinally at a wide range of doses in clinical trials (Bainbridge et al., 2015; Constable et al., 2017; Dimopoulos et al., 2018; Maguire et al., 2009). To investigate whether AAV-induced toxicity is dose-dependent, AAV8-CMV-GFP viruses were injected at 3 doses (5E8 vector particles (vp)/eye, 1E9 vp/eye, and 2E9 vp/eye) into neonatal CD1 mice. The RPE toxicity was evaluated at 30 days post infection. The infected RPE cells displayed a clear correlation between the severity of toxicity and virus dose. A lower dose of 5E8 vp/eye induced RPE cell enlargement with some loss of RPE cells (˜Grade 3), while a higher dose of 2E9 vp/eye caused nearly complete RPE loss (˜Grade 5) (FIG. 4).

Photoreceptor toxicity was examined in preparations where the RPE and retina were kept together, so that neighboring RPE and photoreceptor cells could be inspected for local effects. Cone OS were stained by peanut agglutinin (PNA), which was used as a proxy for overall photoreceptor health. Severe photoreceptor toxicity was seen at the doses of 1E9 and 2E9 vp/eye such that cone OS's largely disappeared (FIGS. 4E, 4F). However, photoreceptors were less sensitive to AAV toxicity than RPE, as AAV8-CMV-GFP-infected RPE demonstrated clear toxicity at the low dose (5E8 vp/eye), while neighboring cone OS's were largely normal (FIGS. 4A and 4D). RPE loss and cone OS loss were usually found in the same area, which could have resulted from higher local infection or an amplifying effect between compromised RPE and photoreceptors. Damage to the RPE and retina was always restricted to the infected area, if a partial infection was seen, as the toxicity did not spread beyond GFP-positive areas (FIGS. 4G-H).

An AAV2 capsid derivative, 7m8, was developed for use in intravitreal injections, i.e. injections into the cavity within the eyeball on the opposite side of the retina from subretinal injections (Dalkara et al., 2013). This injection site is used routinely and safely for the delivery of drugs for age-related macular degeneration (AMD). This capsid was tested using subretinal injections, rather than intravitreal, in order to keep the injection site constant among vectors tested. The hBest1-GFP genome was packaged in AAV2 7m8 and in AAV8 for comparison of two capsid types. The AAV2 7m8 encapsidated stocks did not grow to as high titer in our lab as stocks with other capsid types, so we were only able to inject a maximum dose of 1E8 vp/eye. At this dose, GFP was not seen in as large an area as other capsid types although GFP expression in the infected cells was similar as determined by brightness, and RPE and photoreceptor toxicity was not observed, as analyzed by morphological assays. However, injection of AAV8-hBest1-GFP at the same dose did result in mild toxicity in the RPE (grade 1-2 as well as significant areas where RPE cells were smaller). Cone arrestin overlaying the areas of infection showed a normal pattern. In addition, the area of infection using AAV8-hBest1-GFP was greater than that with AAV2 7m8. The parent capsid type, AAV2, was also tested with genomes that were toxic when encapsidated in AAV8. Injection of AAV2-CMV-null or AAV2-CMV-GFP did not result in RPE or retina toxicity up to 8E8 vp/eye. However, we again noted less infection of the RPE with these vectors relative to infection with these genomes encapsidated in AAV8. The lack of spread using AAV2 has been seen in other tissues, (e.g. Watakabe et al., 2015), perhaps due to AAV2 quickly and effectively binding to cells near the injection site. This may limit the number of cells which are infected with a high enough dose for toxic effects. (FIG. 8).

To further investigate whether toxicity is associated with capsid serotype, particularly type 8, an additional capsid type was tested. AAV-CMV-GFP was packaged in Anc80, a capsid engineered to be less effectively neutralized by prior exposure to extant AAV's (Zinn et al., 2015). AAV toxicity also was seen using Anc80. Previous work conducted with genomes other than those reported here also showed that AAVs capsids induced toxicity (Punzo and Cepko, data not shown).

### AAV Toxicity was Found to be Dependent Upon Vector Structure and not on the Preparation Methods.

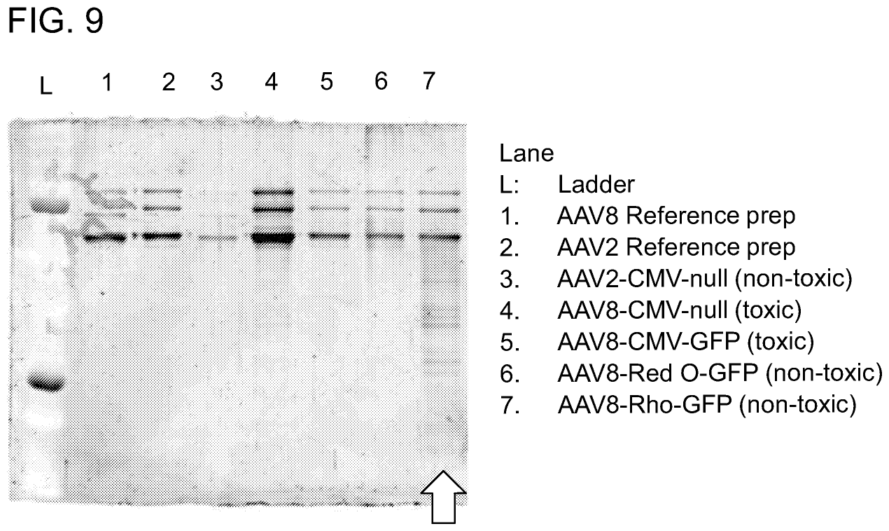

To determine if preparation methods contribute to AAV toxicity, different AAV8 purification protocols, tittering methods, and preparations made by the present lab and three different virus core facilities, were tested. In all cases, toxicity was observed (data not shown). Furthermore, toxic and non-toxic preparations were examined on protein gels to examine the level of contamination by cellular proteins. It was found that cellular protein contaminants did not correlate with toxicity (FIG. 9). The results suggest that the toxicity comes from transgene expression, the capsid proteins, the viral genome structure, and/or unknown contaminants that cannot be assayed. However, the fact that AAV8-CMV-null is toxic argues against protein expression being solely responsible for toxicity. In addition, vectors expressing several other proteins were made, and they were seen to induce toxicity if they used the CMV or CAG promoter (data not shown).

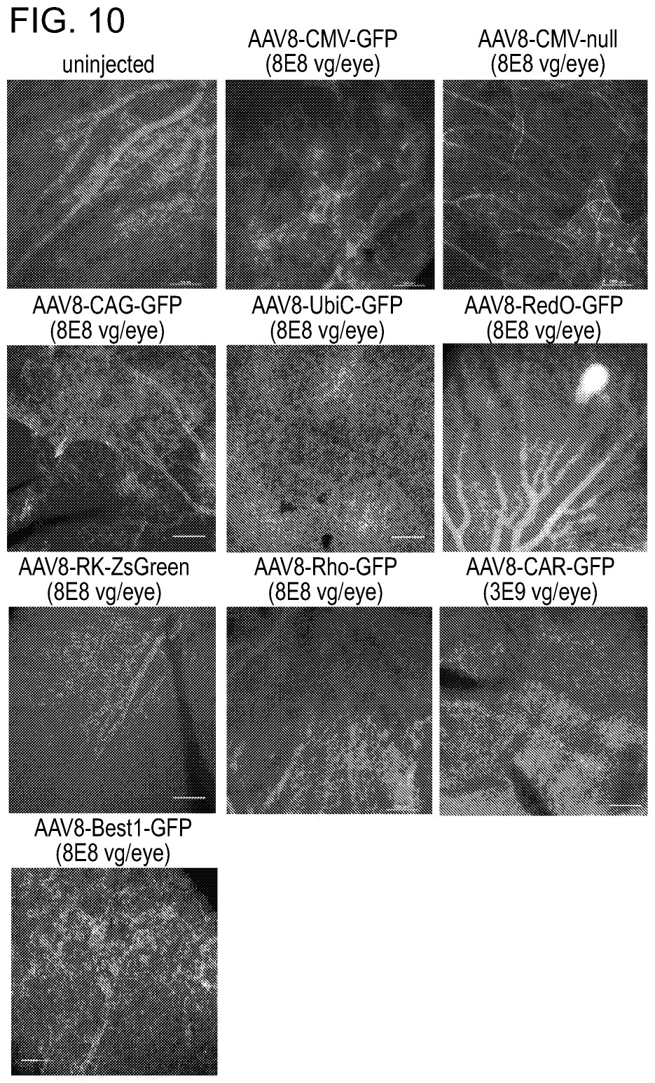

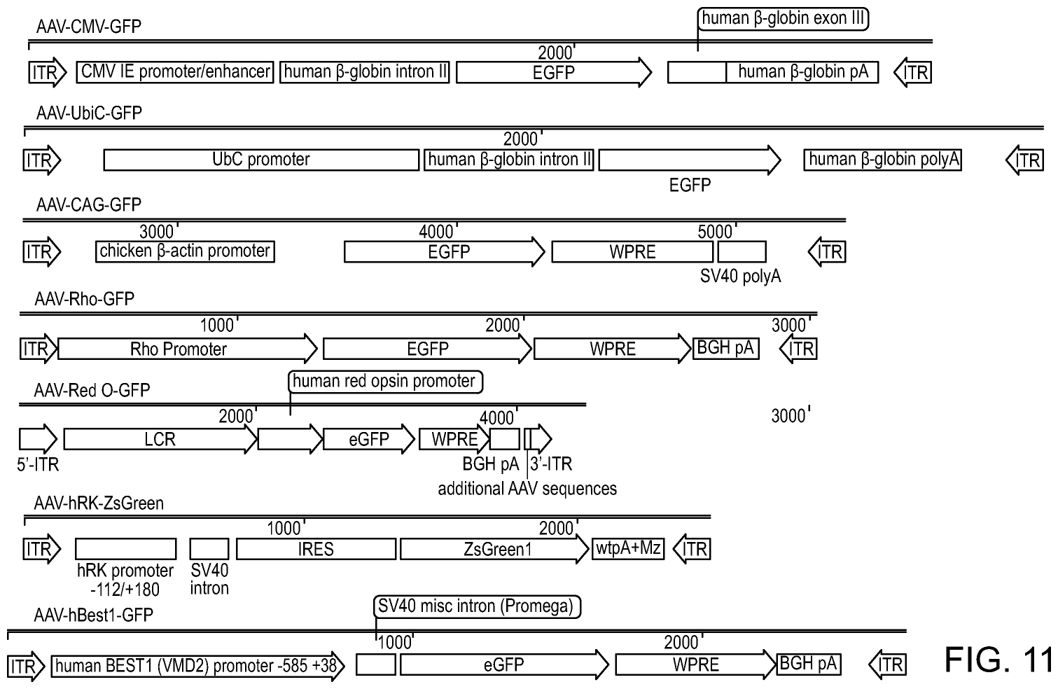

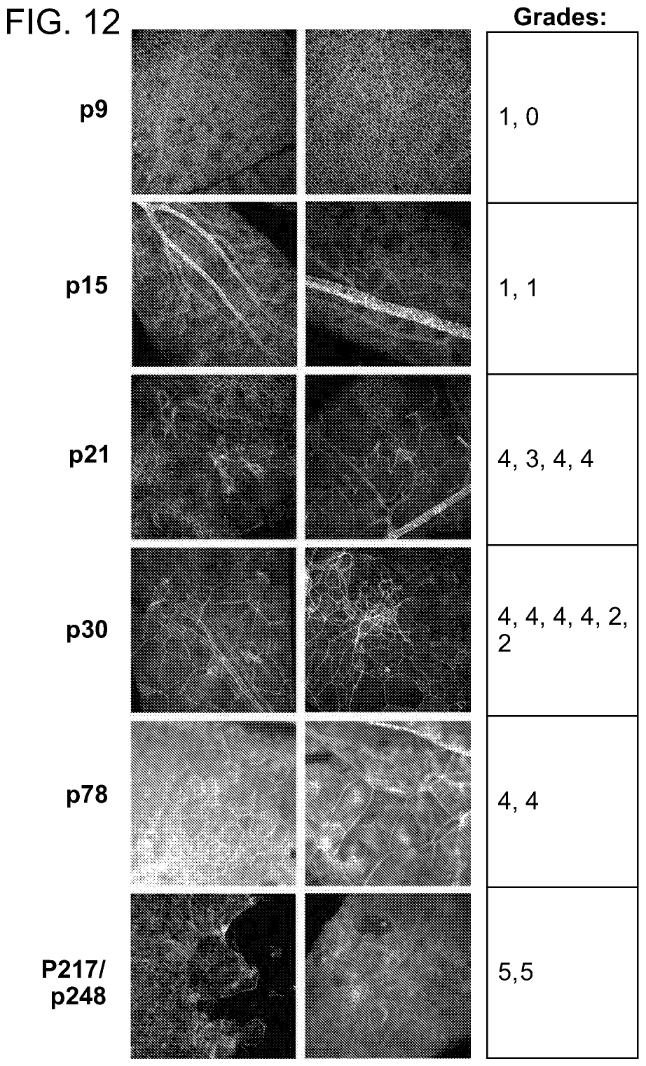

As shown in FIG. 8, there is a strong correlation between toxicity and promoter specificity/sequences. The broadly active promoters, CMV, CAG, and UbiC, as well as the hBest1 promoter were found to be toxic, while the photoreceptor-specific promoters (Red O, Rho, CAR, and hRK) induced no evident toxicity (FIG. 10).

### Assessment of AAV Toxicity Using Clinical Measures of Structure and Visual Activity.

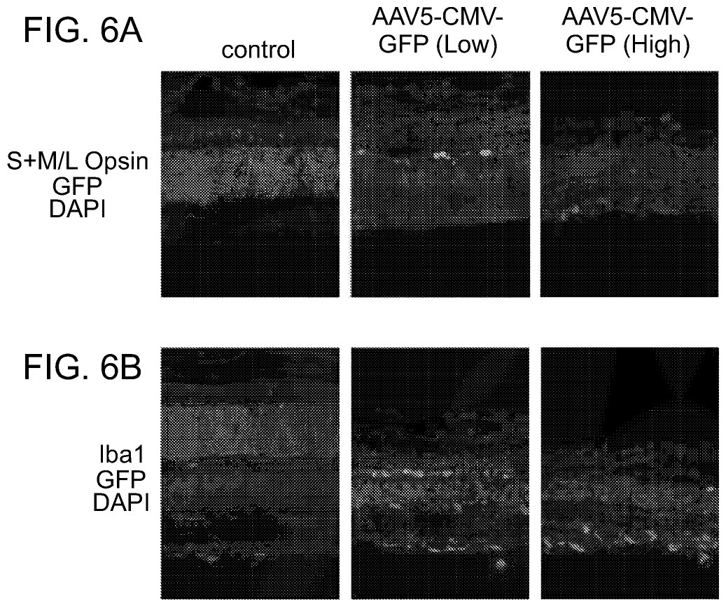

A commonly used assay for the health of human eyes is optical coherence tomography (OCT), an imaging method that can detect alterations in retinal and RPE structure. In addition, human vision can be assayed physiologically using electroretinograms (ERGs), under lighting conditions that assess rod versus cone function. Vision in animals can additionally be measured by a behavioral test, the optomotor assay. These assays were performed on C57BL/6J mice injected with toxic and non-toxic virus preparations at a low dose (8E8 vp/eye) and a high dose (3e9 vp/eye). The C57BL/6J strain was chosen for these assays as it is free of rd8 and cpfl3 mutations which may lead to morphological and functional deficits in the retina (Chang et al., 2006; Mattapallil et al., 2012). The AAV8-CMV-GFP vector was used as a representative of the toxic category. This vector expresses in the RPE, cones, and to a lesser extent, in rods. To compare the results to those from a non-toxic vector preparation with expression in the same cell types, and to test whether toxicity is associated with a serotype other than type 8, a variety of vectors was used: AAV8-CAR-GFP; AAV8-hBest1-GFP for RPE expression, which is non-toxic at low dose, and AAV8-RedO-GFP, at a 1:5 ratio; AAV8-CMV-GFP; and AAV5-CMV-GFP. As an initial measure of infection and to assess potential injection trauma, a fundus camera with a fluorescent light source was used to image GFP and eye morphology. The eyes infected with the non-toxic viruses (AAV8-CAR-GFP, and AAV8-RedO-GFP combined with AAV8-hBesti-GFP) showed uniform expression of GFP in the RPE (FIG. 5A left panels, GFP in photoreceptors was masked by GFP in the RPE). Eyes receiving a low dose of the toxic AAV8-CMV-GFP and AAV5-CMV-GFP virus showed a variable degree of GFP expression in the RPE cells, similar to that observed in cross sections (FIGS. 1 and 6A) and flat-mounts. Fewer GFP+ RPE cells were observed following infection of AAV8-CMV-GFP and AAV5-CMV-GFP at high dose, presumably due to death of RPE cells (FIGS. 5A and 6A). Using OCT to image infected eyes, similar observations were made. Largely normal retinal layers were seen following infection with both low and high doses of the non-toxic preparation (AAV8-CAR-GFP, and AAV8-RedO-GFP combined with AAV8-hBesti-GFP) (FIG. 5B, left panels). In contrast, infection with the low dose of the toxic virus AAV8-CMV-GFP and AAV5-CMV-GFP resulted in diminished outer limiting membrane (OLM) and inner segment/outer segment (IS/OS) bands, created disturbances in the RPE bands, and led to intrusions in the subretinal-RPE space (arrows). Such intrusions may represent infiltrating immune cells (FIG. 5B upper panels). At high dose, infection with the toxic virus AAV8-CMV-GFP and AAV5-CMV-GFP resulted in more dramatic OCT manifestations with decreased ONL and larger subretinal intrusions (FIG. 5B lower panels). Overall, these in vivo imaging results correlate well with our observations of toxic effects on photoreceptors and the RPE seen on ex vivo histological preparations (FIGS. 1, 2, and 6).

All mice that were judged to have successful and non-traumatic injections, as assessed by fluorescent fundus microscopy at ˜P21, were examined by the ERG and optomotor assays at P30. For assessment of rod function and the downstream retinal pathway from rods, scotopic conditions (low light levels without background light) for the ERG were used, while for cone and cone pathway function, photopic conditions (with background light to saturate the rods) were used. The a-wave provides a measure of photoreceptor function, while the b-wave provides a measurement of ON-bipolar cell activity that also indicates the synaptic signaling between photoreceptors and bipolar cells. Injection of the high dose of the toxic virus, AAV8-CMV-GFP, showed a significant drop in the a-wave (−84%, p<0.001) and b-wave amplitudes (−71%, p<0.0001) in scoptopic conditions (FIGS. 5 C and D), suggesting a severe functional deficit in rods and the rod-pathway. Under photopic conditions, the toxic groups showed a 50% to 70% decrease in b-wave amplitudes at all light intensities (FIGS. 5 E and F), compared to the non-toxic group, and a ˜5-fold increase in 1_{1/2} increase (FIG. 5F inset), a measure of the flash intensity giving 50% maximal response (Vinberg et al., 2017). This indicated that the cones or cone-pathway in the toxic group were much less sensitive to light as they required 5-fold more photons to reach the 50% maximal response.

To measure vision, injected mice were tested in the optomotor assay, which measures visual acuity by assessing the motor response of mice under photopic conditions to a virtual rotation of stripes of different widths (Prusky et al., 2004). In keeping with the results of the photopic ERG, the optomotor assay showed a deterioration in photopic vision, with visual acuity decreased by 30% (p<0.05) in the high dose toxic group (FIG. 5G), consistent with a decrease in cone function.

Mice injected with the low dose of the toxic virus had milder perturbations detected by these assays. Several parameters were more comparable to those seen in mice injected with non-toxic viruses including the scotopic ERG b-wave amplitude (FIG. 5D), photopic ERG b-wave amplitudes (FIG. 5F), and photopic optomotor acuity (FIG. 5G). However, subtle visual deficits were still detectable in the low dose toxic virus group: the scotopic ERG a-wave amplitude was 49% lower (p<0.05) than the non-toxic control, and the scotopic b-wave implicit time was significantly delayed, (27 msec slower than control, p<1E-8) (FIG. 5D). In addition, the photopic ERG showed that the 11/2 was—2-fold higher (i.e. 2-fold less sensitive to light) in the low dose toxic group than in the non-toxic virus group (FIG. 5F inset). These results demonstrate that the retinal damage induced by toxic AAV can result in visual deficits, in correlation with the dose of administered virus.

Microglia Activation is Associated with AAV-Induced Toxicity.

Microglia are the main innate immune cell type in the retina. It was determined whether they were activated in retinas infected with toxic AAV at 30 days post virus infection. Iba-1 is a marker of microglia and increases in intensity with activation. Iba-1 staining was examined and it was found that there were significantly more Iba-1 positive microglia in the retina after infection with toxic AAV preps (FIGS. 7A-C). In contrast, microglia number, localization, and morphology did not change significantly in retinas infected with non-toxic AAV preps. Microglia responses were very sensitive indicators of toxicity. In low dose AAV8-CMV-GFP (8E8 vp/eye) infected retinas, in which no clear photoreceptor degeneration was observed, microglia migrated to the ONL and subretinal space, where they adopted an amoeboid or activated morphology (FIGS. 7B-C). To confirm this result, microglia numbers were examined using a transgenic mouse strain, Cx3cr1-GFP, in which microglia are marked by GFP. This strain was injected with AAV8-CMV-TdTomato (3E8 vp/eye), which utilizes the same CMV promoter and human β-globin intron as the toxic AAV8-CMV-GFP. The percentages of all live retinal cells that were GFP+ microglia cells were analyzed using flow cytometry. A three-fold increase of GFP+ microglia was observed in the AAV8-CMV-Tdtomato-infected retinas compared to uninjected or PBS-injected retinas (FIG. 7D).

Activated microglia may increase their expression of pro-inflammatory cytokines, such as tumor necrosis factor alpha (TNFα), interleukin 1 beta (IL-1β), interleukin 6 (IL-6), and/or interferon gamma (IFNγ). This possibility was tested by examining the levels of RNA for TNFα, IL-1β, IL-6, and IFNγ by qPCR in the dissected retinas at 30 days post virus infection. TNFα and IL-1β were highly upregulated in retinas infected with the toxic viruses, while the levels of IL-6 and IFNγ were not significantly changed (FIG. 7E). The increase of TNFα and IL-1β expression correlated with the dose of the injected toxic viruses (FIG. 7E).

### Discussion

Ocular delivery of AAV vectors has been considered to be relatively safe, as shown by the results of several clinical trials. Subretinal injection of AAV is the route used to administer virus for treatment of LCA, retinitis pigmentosa, choroideremia, and neovascular AMD (Constable et al., 2017; Dimopoulos et al., 2018; Ghazi et al., 2016; Maguire et al., 2009). However, the sensitive assays that we were able to conduct in mice have shown that there can be several manifestations of toxicity from subretinal injections of AAV. Toxicity was seen with more than one type of capsid, and did not correlate with preparation methods, endotoxin level, non-viral protein contamination, or mouse strain. The lack of correlation with preparation method is in agreement with the results from previous studies (Hordeaux et al., 2018; Lock et al., 2012). The two variables showing the strongest association to toxicity are the promoters and the viral dose administered. It is likely that other variables can contribute in studies conducted by other groups, e.g. stocks with a high degree of endotoxin are likely to be problematic, but high levels were not seen in our stocks. Our results show that if one develops sensitive assays for different manifestations of toxicity, safer vectors can be developed, reducing the likelihood of problems occurring as a greater number of patients are treated.

In human clinical trials, the dose of AAV used in subretinal injections ranges from 1E10 genome copy (gc)/eye to 1E12 gc/eye (Bainbridge et al., 2015; Constable et al., 2017; Dimopoulos et al., 2018; Maguire et al., 2009). So far, most ocular gene therapy trials have used AAV2, and thus less is known about the safety and efficacy of AAV8 or other capsid types. However, AAV8, and likely other capsid types under development, may offer advantages in terms of number of cells, and/or the cell types, infected. For example, AAV2 did not give as much infection of the RPE as AAV8 in our experiments conducted in mice. AAV8 encapsided toxic genomes were toxic at doses of 5E8 vp/eye to 2E9 vp/eye. Toxic doses for different AAV serotypes are unlikely to be the same, as the cellular tropisms of different AAV serotypes vary greatly (Watanabe et al., 2013). It is difficult to extrapolate across studies, as different subjects/animals, injection routes, and virus tittering methods (genome copies vs viral particles) are used. The vexing issue of differences in titers was well illustrated in a trial where multiple groups measured the titer of the same stock, with differences of up to 100 fold reported (Ayuso et al., 2014). Nonetheless, our results emphasize the importance of testing the dose of specific AAV serotypes with sensitive assays of several phenotypic aspects of relevant cell types, in agreement with the results and recommendations by Hinderer et al. (Hinderer et al., 2018). Most assays conducted to date examine only a few parameters, such as neutralizing antisera or gross inflammation and tissue damage.

In light of clinical trials conducted in the eye, it is interesting that two promoters shown to be toxic in our studies have been used safely in humans. The hBest1 (VMD2) promoter has been used for retinitis pigmentosa, where the RPE gene, MERTK, is defective (Ghazi et al., 2016; Hauswirth et al., 2008). The AAV2-VMD2-hMERTK vector, when administered at 4E8 or 4E9 gc/eye in Sprague-Dawley rats, did not cause any obvious retinal damage compared to the saline injected eyes (Hauswirth et al., 2008). In the follow-up clinical trial, six patients who received either 5.9E10 vg or 1.8E11 vg of AAV2-VMD2-hMERTK vector did not develop severe complications (Ghazi et al., 2016). In our study, however, both AAV8-hBest1-GFP/mApple and AAV2 7m8-hBest1-GFP (8E8 vp/eye) were toxic to the RPE cells in mice Minimizing the level of AAV8-hBest1-GFP to 1E8 vp/eye, as in the non-toxic low dose group, reduced toxicity, while still providing a good level of GFP expression in the RPE (FIG. 5). One interesting observation is that AAV8-hBest1-mApple was less toxic than AAV8-hBest1-GFP (FIG. 3B). It is possible that GFP contributed to the RPE toxicity. In addition to the hBest1 promoter in use in human trials, the CAG promoter is used in the LCA2 vector approved by the FDA (Luxturna). Again, we would suggest that the dose has been chosen to minimize toxicity. However, since subretinal infections of human eyes results in local infection of approximately 10% of retinal or RPE cells, the clinical benefits are more limited than what would be ideal. If safer vectors could be developed, a greater number of cells could be transduced, likely creating greater benefit to the patients, and the concern for safety could be lowered for all injections.

As discussed above, a key observation of our study is that toxicity correlated with promoter type, with the broadly active promoters and an RPE-specific promoter leading to toxicity and photoreceptor-specific ones being benign. Toxicity also has been seen in other tissues with broadly active promoters, and not with cell type-specific promoters (REFs), but a systematic investigation of several of each type has not been reported. One mechanism that might explain toxicity is that the broadly active promoters tend to drive higher expression of transgenes than cell type-specific promoters. GFP protein has been shown to be toxic via reactive oxygen species (ROS), and apoptosis (Ansari et al., 2016; Liu et al., 1999). However, toxicity cannot be solely attributed to GFP or any other protein expression, as AAV8-CMV-null is as toxic as AAV8-CMV-GFP (FIGS. 1 and 2), and other non-GFP proteins were also seen to be toxic when encoded by CMV or CAG vectors (data not shown). Another hypothesis is that the CMV sequence, present in both the CMV and CAG vectors, stimulates an innate immune response, as CMV is a virus that activates the innate immune system naturally. Arguing against this, the UbiC and hBest1 promoters are human in origin, and are also toxic. Another possibility is that there is a common sequence motif among the toxic vectors. Toll-like receptor 9 (TLR9), which senses unmethylated CpG DNA, can detect the AAV genome (Zaiss et al., 2002; Zhu et al., 2009), and can be blocked by inclusion of specific “jamming” sequences within the viral genome (REF). We examined the set of viruses that we tested, and failed to find any correlated sequence motifs. A search for toxic sequences using deletions and chimeric viral genomes may show toxic and/or protective sequences. In addition to innate immune sensing of DNA sequences, capsids also can be sensed, as TLR2, which is on the cell surface, can sense AAV2 and AAV8 capsids in Kupffer cells and liver sinusoidal endothelial cells, but not hepatocytes (Hosel et al., 2012).

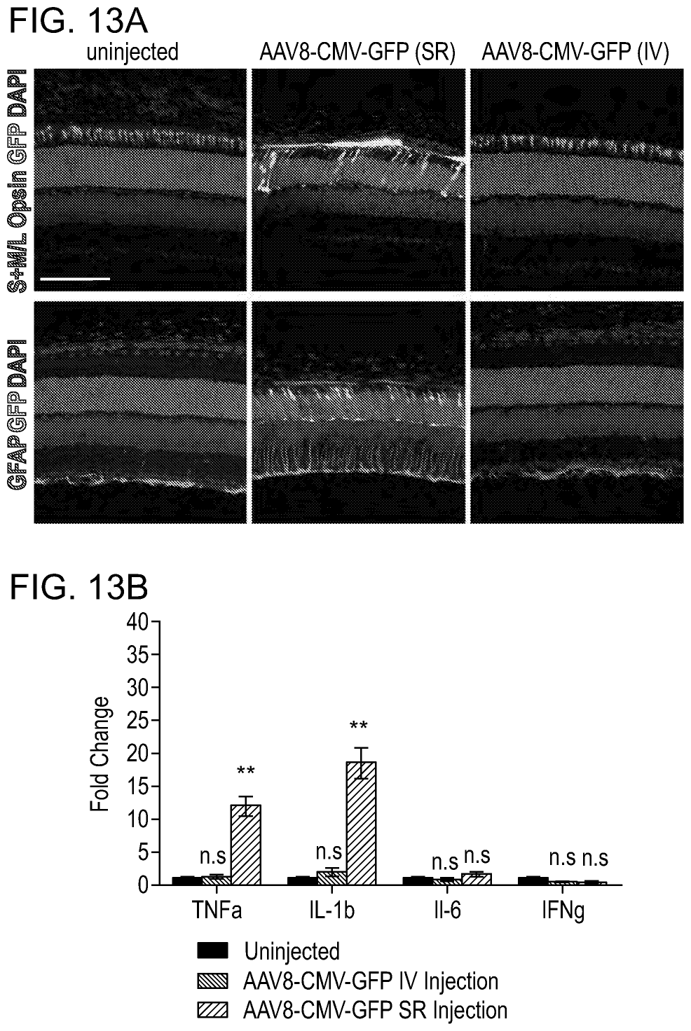

It is of interest to consider the cell types that might be sensing the virus. In addition to microglia, the RPE may sense virus. It is situated between the rich vascular bed of the choriocapillaris and the retina, constitutes a portion of the blood-retinal barrier, and expresses at least several genes of the innate immune system, including the TLRs. Two observations favor a model in which the RPE can be a primary sensor of toxic viruses. AAV vectors with the RPE-specific promoter, hBest1, cause toxicity. In addition, we found that intravitreal injection of a toxic virus, AAV8-CMV-GFP, did not cause any adverse effects (FIG. 13). AAV injected into the vitreous is unlikely to efficiently pass through the retina to the RPE, and infect the RPE at the same level as subretinally delivered virus. The RPE may react when delivered a threshold level of virus, as might also be indicated by the reduced toxicity seen with AAV2 7m8 and AAV2, which gave more limited infection of the RPE. Finally, one can consider the other two retinal glial cell types, Muller glia (MG) and astrocytes, as sensors of virus, as they also can respond to inflammatory stimuli (Jiang et al., 2009, 2012; Kumar and Shamsuddin, 2012). However, Muller glia are not yet born at P0, and astrocytes are just beginning to migrate into the retina at P0 (Kautzman et al., 2018). It is possible that the inflammation seen in other studies of AAV infection of the retina following vitreal injections is due to sensing by either or both of these cell types, or endothelial cells, as the previous studies used injections into mature animals where these cell types would be present and very accessible to vitreally delivered virus.

A novel hypothesis suggested by our data is that it is the act of transcription from a non-chromosomal genome that is being sensed in the RPE and microglia, as toxicity correlates with the promoters that would be active in in the RPE, and possibly microglia. Although we did not see GFP in microglia following infection with any virus, it has been reported that they are difficult to infect in vivo (Rosario et al., 2016). However, it could be that they are infected, but shut down viral gene expression. Detection of viral genomes using the newer and more sensitive DNA FISH methods may resolve this issue (Beliveau et al., 2012; Trotman et al., 2001). The other cell types at the injection site, the retinal neurons, particularly photoreceptors, generally do not express genes encoding sensors of innate immunity (Cherry et al., 2009; Shekhar et al., 2016; Trimarchi et al., 2007, 2008), and thus would not be expected to react directly to viral transcription. If this model should hold, it would be in keeping with both virus dose and promoter activity being correlated with toxicity. Although we have not measured promoter activity level in our study, the levels of GFP fluorescence that were observed in the RPE were considered to be high from all of the toxic promoters.

### Example 2: Development of AAV Expression Cassettes and Constructs that Reduce the Toxicity Associated with AAV while Maintaining Pharmacological Activity

As described in Example 1 above, it was surprisingly found that intraocular delivery, i.e., subretinal delivery, of some AAVs consistently induced cone OS shortening, reduction of the outer nuclear layer (ONL) where rods and cones reside, and dysmorphic RPE, in mice and that this toxicity was correlated with AAV structure. However, as described in the present example, the critical elements have been identified that reduce this toxicity while maintaining pharmacological activity and, based on the identification of these elements therapeutically effective AAV constructs that reduce the toxicity while maintaining pharmacological activity and/or constructs that exhibit benefits of pharmacological activity that outweigh any toxicity associated with the constructs have been developed.

For example, an AAV vector comprising a CMV promoter operably linked to an SV40 intron which is operably linked to a nucleic acid molecule encoding NRF2 or an AAV2/5 vector comprising a CMV promoter operably linked to an SV40 intron which is operably linked to a nucleic acid molecule encoding NRF2 were found to be non-toxic and to inhibit cone cell death in an animal model of RP.

Specifically, as described in U.S. Patent Publication No.: 2016/0279265 [Harvard Reference No. HU 5100; McCarter Reference No. 117823-05402], the entire contents of which are incorporated herein by reference, it has been found that inclusion of Nrf2 in an AAV2/8 construct comprising a CMV promoter preserved cone outer segments in rd1 retinas at P50, prolonged cone survival in rd1 retinas at P50, and preserved cone function as assessed by optomotor assay, electroretinography, and light-evoked ganglion cell activity in mouse models of retinitis pigmentosa.

### Example 3: Development of AAV Expression Cassettes and Constructs that Reduce the Toxicity Associated with AAV and Maintain Pharmacological Activity

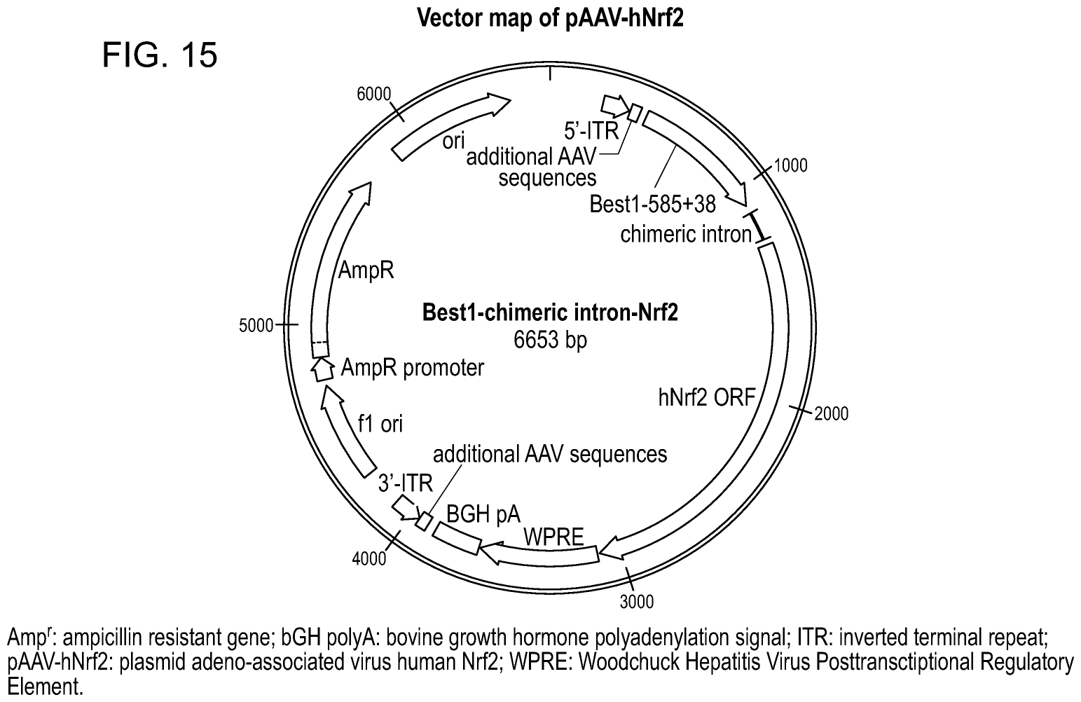

An AAV vector comprising the toxic promoter hBEST1, a chimeric intron and Nrf2 was also prepared and assessed for retinal pharmacology. An exemplary expression cassette of this construct is depicted in FIG. 14 and an exemplary vector map of such a construct (referred to herein as pAAV-hNrf2) is depicted in FIG. 15.

Briefly, the 293T cell line was thawed and expanded in the culture vessels, and then the cells were transfected with the three purified plasmid DNAs (pAAV-hNrf2, pRep2Cap8 and pAAV-Helper). After production culturing, Triton X-100 was added to the 293T cell culture and AAV8-hNrf2 was collected as crude vector extract solution. For rough purification of crude vector extract solution, PEG precipitation was performed. Affinity chromatography with POROS™ CaptureSelect™ AAV8 Affinity Resin (Thermo Fisher Scientific: Product code A30790) was performed, and fractions containing AAV8-hNrf2 were collected. Next, ultracentrifugation with cesium chloride was performed, the solution was fractionated from the top of the tube and fractions containing AAV8-hNrf2 were collected. After two dialysis was performed for buffer exchange, the purified AAV8-hNrf2 as drug substance was produced by sterile filtration. The titer of purified AAV8-hNrf2 was quantified using AAVpro Titration Kit Ver. 2 (Takara Bio Inc.: Product code 6233).

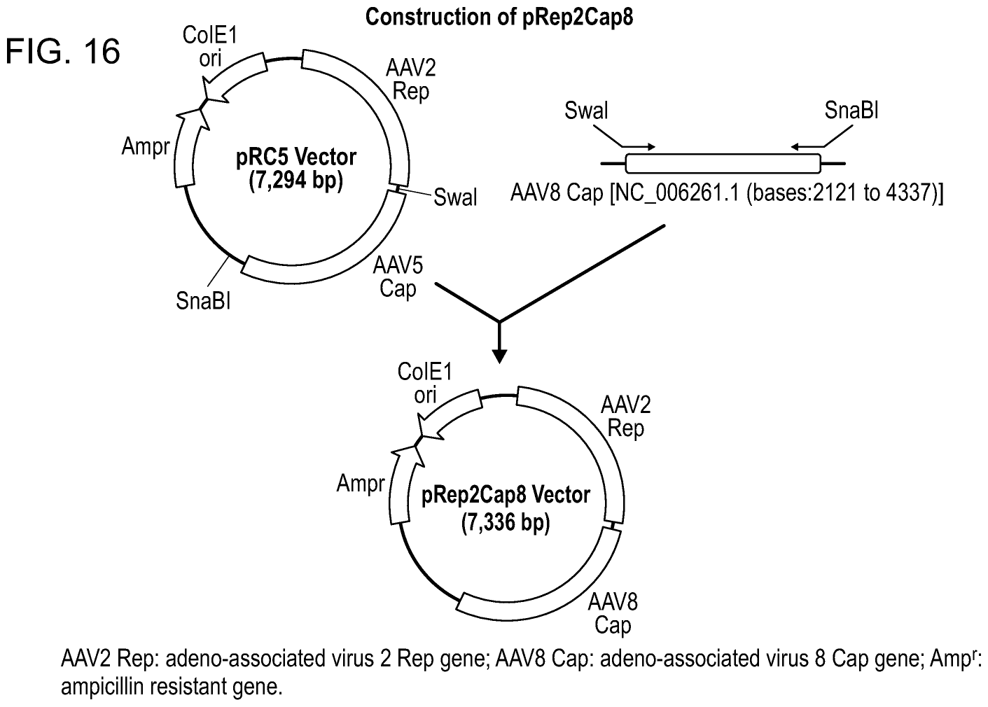

Construction of the the pRep2Cap8 vector loaded with an AAV packaging molecule is a vector plasmid containing a Rep2 gene related to replication and transcription of AAV2 and a Cap8 gene (AAV8 Cap) that codes the coat protein of a virus particle. AAV8 Cap sequence [NCBI Reference Sequence: NC_006261.1 (bases: 2121 to 4337)] was synthesized and replaced from the Cap5 in the pRC5 Vector (Takara Bio Inc.: Product code 6664) to construct pRep2Cap8 (FIG. 16).

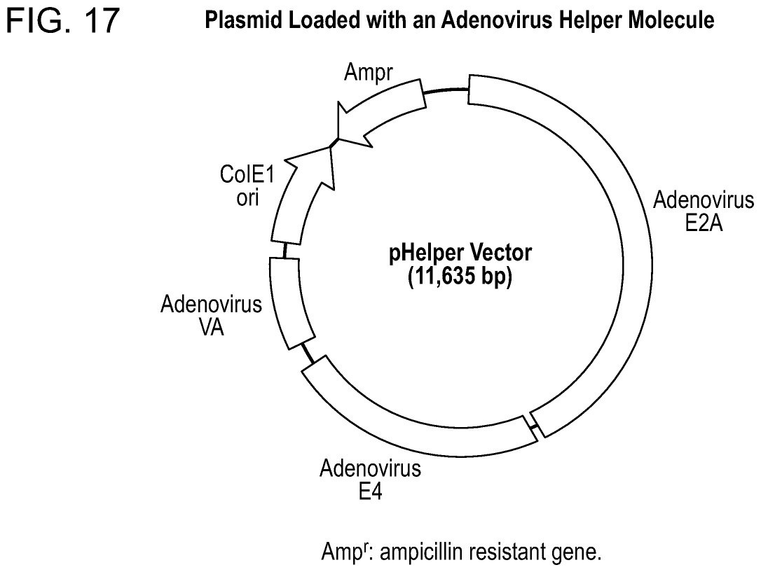

The pAAV-Helper Vector loaded with an adenovirus helper molecule (Takara Bio Inc.: Product code 6230) is a vector plasmid containing E2A (Early 2A), E4 (Early 4) and VA (viral associated) derived from an adenovirus (FIG. 17).

In order to further assess the effect of this AAV construct, B6.CXB1-Pde6brd10/J, rd10 mice, one of the most used models of RP was selected for these analyses. As PDE6b (rod-phosphodiesterase (PDE) 6b) is the gene that is mutated in some RP patients and this mouse model carries a spontaneous mutation of the Pde6b gene and develops progressive photoreceptor degeneration, this model closely mimics the cause and pathogenesis of RP in humans. Although it takes about 7 weeks for Nrf2 transgene's expression by AAV8 to reach a plateau after subretinal injection (Natkunarajah, Trittibach et al. 2008, Gene. Ther. 15, 463-467), this mouse models shows the progressive pathological condition that can be used evaluate the efficacy of the AAV construct and, thus, was selected as a pivotal animal model.

Accordingly, this AAV construct was subretinally injected on post-natal day 1 (PND 1) and optomotor response was measured on PNDs 30, 50, 70, 90, and 108. In addition, photopic ERG was assessed on PND 35 and opsin count, as a cone marker, was assessed on PND 50.

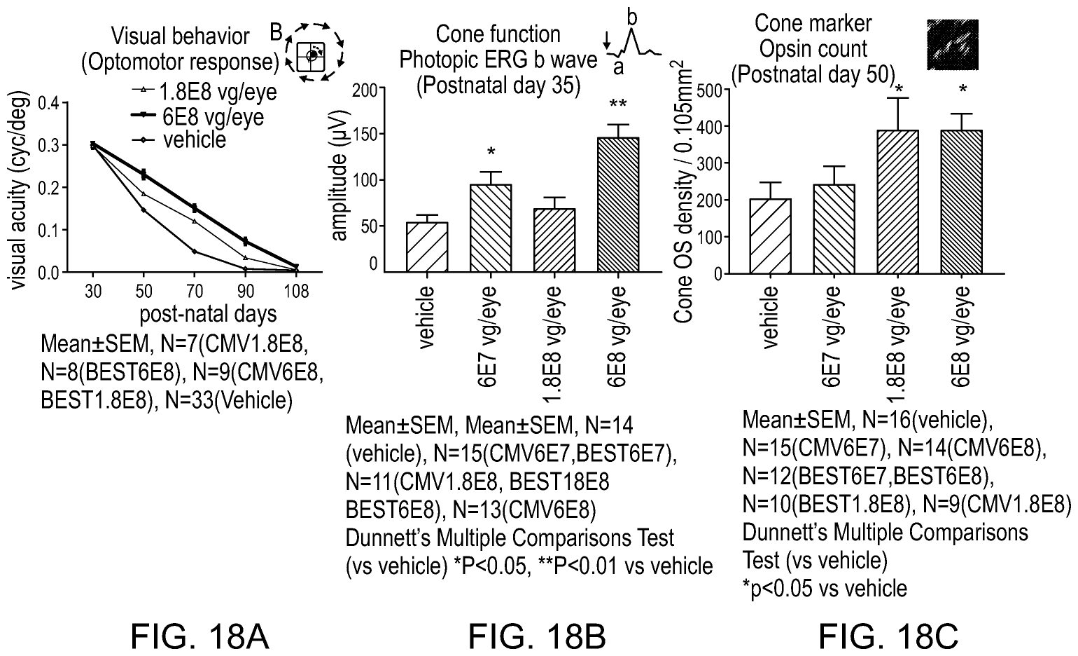

Administration of this AAV construct at a dose of 6E8 viral genomes (vg)/eye delayed progression of loss of functional vision as assessed by optomotor response (FIG. 18A), photopic ERG (FIG. 18B), and cone marker (FIG. 18C) compared with the vehicle group and administration of this AAV construct at a dose of 1.8E8 vg/eye delayed progression of loss of functional vision as assessed by optomotor response and cone marker compared with the vehicle group. In this study, the effective dose in mouse model was 1.8E8-6E8 vg/eye.

### REFERENCES

Acland, G. M., Aguirre, G. D., Ray, J., Zhang, Q., Aleman, T. S., Cideciyan, a V, Pearce-Kelling, S. E., Anand, V., Zeng, Y., Maguire, a M., et al. (2001). Gene therapy restores vision in a canine model of childhood blindness. Nat. Genet. 28, 92-95.
Allocca, M., Mussolino, C., Garcia-Hoyos, M., Sanges, D., lodice, C., Petrillo, M., Vandenberghe, L. H., Wilson, J. M., Marigo, V., Surace, E. M., et al. (2007). Novel adeno-associated virus serotypes efficiently transduce murine photoreceptors. J. Virol. 81, 11372-11380.
Ansari, A. M., Ahmed, A. K., Matsangos, A. E., Lay, F., Born, L. J., Marti, G., Harmon, J. W., and Sun, Z. (2016). Cellular GFP Toxicity and Immunogenicity: Potential Confounders in in Vivo Cell Tracking Experiments. Stem Cell Rev. Reports 12, 553-559.
Ayuso, E., Blouin, V., Lock, M., McGorray, S., Leon, X., Alvira, M. R., Auricchio, A., Bucher, S., Chtarto, A., Clark, K. R., et al. (2014). Manufacturing and Characterization of a Recombinant Adeno-Associated Virus Type 8 Reference Standard Material. Hum. Gene Ther. 25, 977-987.
Bainbridge, J. W. B., Smith, A. J., Barker, S. S., Robbie, S., Henderson, R., Balaggan, K., Viswanathan, A., Holder, G. E., Stockman, A., Tyler, N., et al. (2008). Effect of gene therapy on visual function in Leber's congenital amaurosis. N. Engl. J. Med. 358, 2231-2239.
Bainbridge, J. W. B., Mehat, M. S., Sundaram, V., Robbie, S. J., Barker, S. E., Ripamonti, C., Georgiadis, A., Mowat, F. M., Beattie, S. G., Gardner, P. J., et al. (2015). Long-Term Effect of Gene Therapy on Leber's Congenital Amaurosis. N. Engl. J. Med. 150504083137004.
Beliveau, B. J., Joyce, E. F., Apostolopoulos, N., Yilmaz, F., Fonseka, C. Y., McCole, R. B., Chang, Y., Li, J. B., Senaratne, T. N., Williams, B. R., et al. (2012). Versatile design and synthesis platform for visualizing genomes with Oligopaint FISH probes. Proc. Natl. Acad. Sci. 109, 21301-21306.
Busskamp, V., Duebel, J., Balya, D., Fradot, M., Viney, T. J., Groner, A. C., Cabuy, E., Seeliger, M., Biel, M., Paques, M., et al. (2010). Genetic reactivation of cone photoreceptors restores visual responses in retinitis pigmentosa. Supporting Material.
Chang, B., Dacey, M. S., Hawes, N. L., Hitchcock, P. F., Milam, A. H., Atmaca-Sonmez, P., Nusinowitz, S., and Heckenlively, J. R. (2006). Cone photoreceptor function loss-3, a novel mouse model of achromatopsia due to a mutation in Gnat2. Invest. Ophthalmol. Vis. Sci. 47, 5017-5021.
Cherry, T. J., Trimarchi, J. M., Stadler, M. B., and Cepko, C. L. (2009). Development and diversification of retinal amacrine interneurons at single cell resolution. Proc. Natl. Acad. Sci. 106, 9495-9500.
Constable, I. J., Lai, C. M., Magno, A. L., French, M. A., Barone, S. B., Schwartz, S. D., Blumenkranz, M. S., Degli-Esposti, M. A., and Rakoczy, E. P. (2017). Gene Therapy in Neovascular Age-related Macular Degeneration: Three-Year Follow-up of a Phase 1 Randomized Dose Escalation Trial. Am. J. Ophthalmol./77,150-158.
Dalkara, D., Byrne, L. C., Klimczak, R. R., Visel, M., Yin, L., Merigan, W. H., Flannery, J. G., and Schaffer, D. V. (2013). In Vivo-Directed Evolution of a New Adeno-Associated Virus for Therapeutic Outer Retinal Gene Delivery from the Vitreous. Sci. Transl. Med. 5, 189ra76-189ra76.
Dimopoulos, I. S., Hoang, S. C., Radziwon, A., Binczyk, N. M., Seabra, M. C., MacLaren, R. E., Somani, R., Tennant, M. T. S., and MacDonald, I. M. (2018). Two-Year Results after AAV2-Mediated Gene Therapy for Choroideremia: The Alberta Experience. Am. J. Ophthalmol. 193, 130-142.
Dixon, C. M., Kusek, J. W., Ph, D., Lepor, H., Mcvary, K. T., Nyberg, L. M., Ph, D., Clarke, H. S., Ph, D., Crawford, E. D., et al. (2011). Adenovirus-Associated Virus Vector—Mediated Gene Transfer in Hemophilia B. Engl. J. Med. 365,2357-2365.
Esumi, N., Oshima, Y., Li, Y., Campochiaro, P. A., and Zack, D. J. (2004). Analysis of the VMD2 Promoter and Implication of E-box Binding Factors in Its Regulation. J. Biol. Chem. 279,19064-19073.
Gao, G., Vandenberghe, L. H., Alvira, M. R., Lu, Y., Calcedo, R., Zhou, X., and Wilson, J. M. (2004). Clades of Adeno-Associated Viruses Are Widely Disseminated in Human Tissues. J. Virol. 78, 6381-6388.
Ghazi, N. G., Abboud, E. B., Nowilaty, S. R., Alkuraya, H., Alhommadi, A., Cai, H., Hou, R., Deng, W. T., Boye, S. L., Almaghamsi, A., et al. (2016). Treatment of retinitis pigmentosa due to MERTK mutations by ocular subretinal injection of adeno-associated virus gene vector: results of a phase I trial. Hum. Genet. 135, 327-343.
Gootwine, E., Ofri, R., Banin, E., Obolensky, A., Averbukh, E., Ezra-Elia, R., Ross, M., Honig, H., Rosov, A., Yamin, E., et al. (2017). Safety and Efficacy Evaluation of rAAV2tYF-PR1.7-hCNGA3 Vector Delivered by Subretinal Injection in CNGA3 Mutant Achromatopsia Sheep. Hum. Gene Ther. Clin. Dev. 28, 96-107.
Grieger, J. C., Choi, V. W., and Samulski, R. J. (2006). Production and characterization of adeno-associated viral vectors. Nat. Protoc. 1, 1412-1428.
Hauswirth, W. W., Aleman, T. S., Kaushal, S., Cideciyan, A. V, Schwartz, S. B., Wang, L., Conlon, T. J., Boye, S. L., Flotte, T. R., Byrne, B. J., et al. (2008). Treatment of leber congenital amaurosis due to RPE65 mutations by ocular subretinal injection of adeno-associated virus gene vector: short-term results of a phase I trial. Hum. Gene Ther. 19, 979-990.
Hinderer, C., Katz, N., Buza, E. L., Dyer, C., Goode, T., Bell, P., Richman, L., and Wilson, J. M. (2018). Severe toxicity in nonhuman primates and piglets following high-dose intravenous administration of an AAV vector expressing human SMN. Hum. Gene Ther. hum. 2018.015.
Hordeaux, J., Wang, Q., Katz, N., Buza, E. L., Bell, P., and Wilson, J. M. (2018). The Neurotropic Properties of AAV-PHP.B Are Limited to C57BL/6J Mice. Mol. Ther. 26, 664-668.
Hosel, M., Broxtermann, M., Janicki, H., Esser, K., Arzberger, S., Hartmann, P., Gillen, S., Kleeff, J., Stabenow, D., Odenthal, M., et al. (2012). Toll-like receptor 2-mediated innate immune response in human nonparenchymal liver cells toward adeno-associated viral vectors. Hepatology 55, 287-297.
Jiang, G., Ke, Y., Sun, D., Wang, Y., Kaplan, H. J., and Shao, H. (2009). Regulatory role of TLR ligands on the activation of autoreactive T cells by retinal astrocytes. Investig. Ophthalmol. Vis. Sci. 50, 4769-4776.
Jiang, G., Sun, D., Kaplan, H. J., and Shao, H. (2012). Retinal astrocytes pretreated with NOD2 and TLR2 ligands activate uveitogenic t cells. PLoS One 7,1-9.
Jung, S., Aliberti, J., Graemmel, P., Sunshine, M. J., Kreutzberg, G. W., Sher, A., and Littman, D. R. (2000). Analysis of Fractalkine Receptor CX3CR1 Function by Targeted Deletion and Green Fluorescent Protein Reporter Gene Insertion. Mol. Cell. Biol. 20, 4106-4114.
Kautzman, A. G., Keeley, P. W., Nahmou, M. M., Luna, G., Fisher, S. K., and Reese, B. E. (2018). Sox2 regulates astrocytic and vascular development in the retina. Glia 66, 623-636.
Khani, S. C., Pawlyk, B. S., Bulgakov, 0. V., Kasperek, E., Young, J. E., Adamian, M., Sun, X., Smith, a. J., Ali, R. R., and Li, T. (2007). MV-Mediated Expression Targeting of Rod and Cone Photoreceptors with a Human Rhodopsin Kinase Promoter. Invest. Ophthalmol. Vis. Sci. 48, 3954-3961.
Kumar, A., and Shamsuddin, N. (2012). Retinal muller glia initiate innate response to infectious stimuli via toll-like receptor signaling. PLoS One 7.
Liu, H.-S., Jan, M.-S., Chou, C.-K., Chen, P.-H., and Ke, N.-J. (1999). Is Green Fluorescent Protein Toxic to the Living Cells? Biochem. Biophys. Res. Commun. 260, 712-717.
Lock, M., Alvira, M. R., and Wilson, J. M. (2012). Analysis of Particle Content of Recombinant Adeno-Associated Virus Serotype 8 Vectors by Ion-Exchange Chromatography. Hum. Gene Ther. Methods 23,56-64.
MacLaren, R. E., Groppe, M., Barnard, A. R., Cottriall, C. L., Tolmachova, T., Seymour, L., Reed Clark, K., During, M. J., Cremers, F. P. M., Black, G. C. M., et al. (2014). Retinal gene therapy in patients with choroideremia: Initial findings from a phase 1/2 clinical trial. Lancet 383,1129-1137.
Maguire, A. M., Simonelli, F., Pierce, E. A., Pugh, E. N., Mingozzi, F., Bennicelli, J., Banfi, S., Marshall, K. A., Testa, F., Arruda, V. R., et al. (2008). Safety and Efficacy of Gene Transfer for Leber's Congenital Amaurosis. N. Engl. J. Med.
Maguire, A. M., High, K. A., Auricchio, A., Wright, J. F., Pierce, E. A., Testa, F., Mingozzi, F., Bennicelli, J. L., Ying, G. shuang, Rossi, S., et al. (2009). Age-dependent effects of RPE65 gene therapy for Leber's congenital amaurosis: a phase 1 dose-escalation trial. Lancet 374, 1597-1605.
Matsuda, T., and Cepko, C. L. (2004). Electroporation and RNA interference in the rodent retina in vivo and in vitro. Proc. Natl. Acad. Sci. 101, 16-22.
Mattapallil, M. J., Wawrousek, E. F., Chan, C.-C., Zhao, H., Roychoudhury, J., Ferguson, T. A., and Caspi, R. R. (2012). The Rd8 Mutation of the Crb1 Gene Is Present in Vendor Lines of C57BL/6N Mice and Embryonic Stem Cells, and Confounds Ocular Induced Mutant Phenotypes. Invest. Ophthalmol. Vis. Sci. 53,2921-2927.
Mingozzi, F., and High, K. a (2013). Immune responses to AAV vectors: overcoming barriers to successful gene. Blood 122, 23-36.
Prusky, G. T., Alam, N. M., Beekman, S., and Douglas, R. M. (2004). Rapid quantification of adult and developing mouse spatial vision using a virtual optomotor system. Invest. Ophthalmol. Vis. Sci. 45, 4611-4616.
Rosario, A. M., Cruz, P. E., Ceballos-Diaz, C., Strickland, M. R., Siemienski, Z., Pardo, M., Schob, K. L., Li, A., Aslanidi, G. V., Srivastava, A., et al. (2016). Microglia-specific targeting by novel capsid-modified AAV6 vectors. Mol. Ther.-Methods Clin. Dev. 3,16026.
Shekhar, K., Lapan, S. W., Whitney, I. E., Tran, N. M., Macosko, E. Z., Kowalczyk, M., Adiconis, X., Levin, J. Z., Nemesh, J., Goldman, M., et al. (2016). Comprehensive Classification of Retinal Bipolar Neurons by Single-Cell Transcriptomics. Cell 166, 1308-1323.e30.
Trimarchi, J. M., STADLER, M. B., ROSKA, B., BILLINGS, N., SUN, B., BRANDON BARTCH, and CEPKO, A. C. L. (2007). Molecular Heterogeneity of Developing Retinal Ganglion and Amacrine Cells Revealed through Single Cell Gene Expression Profiling. J Comp Neurol 502, 1047-1065.
Trimarchi, J. M., Stadler, M. B., and Cepko, C. L. (2008). Individual retinal progenitor cells display extensive heterogeneity of gene expression. PLoS One 3.
Trotman, L. C., Mosberger, N., Fornerod, M., Stidwill, R. P., and Greber, U. F. (2001). Import of adenovirus DNA involves the nuclear pore complex receptor CAN/Nup214 and histone H1. Nat. Cell Biol. 3.
Vandenberghe, L. H., Xiao, R., Lock, M., Lin, J., Korn, M., and Wilson, J. M. (2010). Efficient serotypedependent release of functional vector into the culture medium during adeno-associated virus manufacturing. Hum. Gene Ther. 21, 1251-1257.
Vinberg, F., Wang, T., De Maria, A., Zhao, H., Bassnett, S., Chen, J., and Kefalov, V. J. (2017). The Na(+)/Ca(2+), K(+) exchanger NCKX4 is required for efficient cone-mediated vision. Elife 6, e24550.
Wang, S., Sengel, C., Emerson, M. M., and Cepko, C. L. (2014). A gene regulatory network controls the binary fate decision of rod and bipolar cells in the vertebrate retina. Dev. Cell 30, 513-527.
Wang, Y., Macke, J. P., Merbs, S. L., Zack, D. J., Klaunberg, B., Bennett, J., Gearhart, J., and Nathans, J. (1992). A locus control region adjacent to the human red and green visual pigment genes. Neuron 9, 429-440.
Watakabe, A., Ohtsuka, M., Kinoshita, M., Takaji, M., Isa, K., Mizukami, H., Ozawa, K., Isa, T., and Yamamori, T. (2015). Comparative analyses of adeno-associated viral vector serotypes 1, 2, 5, 8 and 9 in marmoset, mouse and macaque cerebral cortex. Neurosci. Res. 93,144-157.
Watanabe, S., Sanuki, R., Ueno, S., Koyasu, T., Hasegawa, T., and Furukawa, T. (2013). Tropisms of AAV for subretinal delivery to the neonatal mouse retina and its application for in vivo rescue of developmental photoreceptor disorders. PLoS One 8, e54146.
Xiong, W., Garfinkel, A. E. M., Li, Y., Benowitz, L. I., and Cepko, C. L. (2015). NRF2 promotes neuronal survival in neurodegeneration and acute nerve damage. J. Clin. Invest. 125, 1433-1445.
Xue, Y., Shen, 5.0., Jui, J., Rupp, A. C., Byrne, L. C., Hattar, S., Flannery, J. G., Corbo, J. C., and Kefalov, V. J. (2015a). CRALBP supports the mammalian retinal visual cycle and cone vision. J. Clin. Invest. 125, 727-738.
Xue, Y., Shen, 5.0., Corbo, J. C., and Kefalov, V. J. (2015b). Circadian and light-driven regulation of rod dark adaptation. Sci. Rep. 5,1-10.
Xue, Y., Sato, S., Razafsky, D., Sahu, B., Shen, S. Q., Potter, C., Sandell, L. L., Corbo, J. C., Palczewski, K., Maeda, A., et al. (2017). The role of retinol dehydrogenase 10 in the cone visual cycle. Sci. Rep. 7,1-13.
Zaiss, A.-K., Liu, Q., Bowen, G. P., Wong, N. C. W., Bartlett, J. S., and Muruve, D. A. (2002).
Differential activation of innate immune responses by adenovirus and adeno-associated virus vectors. J. Virol. 76, 4580-4590.
Zhu, J., Huang, X., and Yang, Y. (2016). The TLR9-MyD88 pathway is critical for adaptive immune responses to adeno-associated virus gene therapy vectors in mice. J. Clin. Invest. 119, 2388-2398.
Zhu X, Ma B, Babu S, Murage J, Knox B E, Craft C M. Mouse cone arrestin gene characterization: Promoter targets expression to cone photoreceptors. FEBS Lett. 2002; 524(1-3):116-22.
Zinn, E., Pacouret, S., Khaychuk, V., Turunen, H. T., Carvalho, L. S., Andres-Mateos, E., Shah, S.,
Shelke, R., Maurer, A. C., Maurer, E., et al. (2015). In silico reconstruction of the viral evolutionary lineage yields a potent gene therapy vector. Cell Rep. 12, 1056-1068.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A composition, comprising an adeno-associated virus (AAV) expression cassette, the expression cassette comprising a human bestrophin 1 (hBest1) promoter, a chimeric intron, and a nucleic acid molecule encoding nuclear factor erythroid 2-like 2 (Nrf2).

2. The composition of claim 1, wherein the hBest1 promoter comprises nucleotides −585 to +38 of the hBest1gene; nucleotides −154 to +38 of the hBest1 gene; or nucleotides −104 to +38 bp of the hBest1 gene, or or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides −585 to +38 of the hBest1gene; nucleotides −154 to +38 of the hBest1 gene; or nucleotides −104 to +38 bp of the hBest1 gene.

3.-7. (canceled)

8. The composition of claim 1, wherein the chimeric intron comprises a 5′-donor site from the first intron of the human β-globin gene and the branch and 3′-acceptor site from the intron that is between the leader and the body of an immunoglobulin gene heavy chain variable region.

9. (canceled)

10. The composition of claim 1, wherein the expression cassette further comprises a post-transcriptional regulatory region.

11. The composition of claim 1, wherein the expression cassette further comprises a Woodchuck hepatitis virus posttranscriptional regulatory element (WPRE).

12. The composition of claim 1, wherein the expression cassette further comprises a post-transcriptional regulatory region comprising nucleotides 3110-3651 of the nucleotide sequence in FIG. 19 (SEQ ID NO:21), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3110-3651 of the nucleotide sequence in FIG. 19 (SEQ ID NO:21).

13. The composition of claim 1, wherein the expression cassette further comprises a post-transcriptional regulatory region comprising the nucleotide sequence of SEQ ID NO: 18, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 18.

14. The composition of claim 1, wherein the expression cassette is present in a vector.

15. (canceled)

16. An AAV vector particle comprising the composition of claim 1.

17. An isolated cell comprising the AAV particle of claim 16.

18. A pharmaceutical composition comprising the AAV composition of claim 1 or the particle of claim 16.

19.-21. (canceled)

22. A method for prolonging the viability of a photoreceptor cell compromised by a degenerative ocular disorder, comprising contacting said cell with the composition of claim 1, the AAV viral particle of claim 16, or the pharmaceutical composition of claim 18, thereby prolonging the viability of the photoreceptor cell compromised by the degenerative ocular disorder.

23. A method for treating or preventing a degenerative ocular disorder in a subject, comprising administering to said subject a therapeutically effective amount of the composition of claim, the AAV viral particle of claim 16, or the pharmaceutical composition of claim 18, thereby treating or preventing said degenerative ocular disorder.

24.-30. (canceled)


SEQUENCE LISTING (26 sequences)

SEQ ID NO: 1 - DNA, 2859 bp, Homo sapiens
aaatcagggaggcgcagctcctacaccaacgcctttccggggctccgggtgtgtttgttccaactgtttaaactgtttcaaagcgtccgaactccagcgaccttcgcaaacaactctttatctcgcgggcgagagcgctgcccttatttgcgggggagggcaaactgaacgccggcaccggggagctaacggagacctcctctaggtcccccgcctgctgggaccccagctggcagtcccttcccgcccccggaccgcgagcttcttgcgtcagccccggcgcgggtgggggattttcggaagctcagcccgcgcggccggcgggggaaggaagggcccggactcttgccccgcccttgtggggcgggaggcggagcggggcaggggcccgccggcgtgtagccgattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 2 - DNA, 2918 bp, Homo sapiens
tcgggggaactgaggtcgccttcgggctgggacccggagccccttcgccgcgccccaagacctccttgagtgcgggctgcgacgcgctcaccccgctgggccgtctgtgggcgcggctttgcgaagtcatccatctctcggatcactctctggcagccttgagctctcttgaaagcccagccccgggacgagggaggagcgccttaagtgcccagcgggctcagaagccccgacgtgtggcggctgagccgggccccgcgcactttctcggccggggaggggttcgggctcgggcacccggagttggcccctcgtaacgccgcgggaaagtgcgggcgagggcagtggactctgaggccggagtcggcggcacccggggcttctagttcggacgcggtgccccctggtggcgctcaccgcgcgcgtggccttggcttccgtgacagcgctcggttggccgtcacagcagccctcggttggccctttcctgctttatagcgtgcaaacctcgccgcgccagggccaagggacaggttggagctgttgatctgttgcgcaattgctattttccccagagcggctttgtctttggatttagcgtttcagaattgcaattccaaaatgtgtaagacgggatattctcttctgtgctgtcaagggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 3 - DNA, 2967 bp, Homo sapiens
ggcccttccggggctgcgcggctcccccgcctcggtgccggcaaaaatgtgcctagtcacggggccgctctcgggggaactgaggtcgccttcgggctgggacccggagccccttcgccgcgccccaagacctccttgagtgcgggctgcgacgcgctcaccccgctgggccgtctgtgggcgcggctttgcgaagtcatccatctctcggatcactctctggcagccttgagctctcttgaaagcccagccccgggacgagggaggagcgccttaagtgcccagcgggctcagaagccccgacgtgtggcggctgagccgggccccgcgcactttctcggccggggaggggttcgggctcgggcacccggagttggcccctcgtaacgccgcgggaaagtgcgggcgagggcagtggactctgaggccggagtcggcggcacccggggcttctagttcggacgcggtgccccctggtggcgctcaccgcgcgcgtggccttggcttccgtgacagcgctcggttggccgtcacagcagccctcggttggccctttcctgctttatagcgtgcaaacctcgccgcgccagggccaagggacaggttggagctgttgatctgttgcgcaattgctattttccccagagcggctttgtctttggatttagcgtttcagaattgcaattccaaaatgtgtaagacgggatattctcttctgtgctgtcaagggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 4 - DNA, 2862 bp, Homo sapiens
ggcccttccggggctgcgcggctcccccgcctcggtgccggcaaaaatgtgcctagtcacggggccgctctcgggggaactgaggtcgccttcgggctgggacccggagccccttcgccgcgccccaagacctccttgagtgcgggctgcgacgcgctcaccccgctgggccgtctgtgggcgcggctttgcgaagtcatccatctctcggatcactctctggcagccttgagctctcttgaaagcccagccccgggacgagggaggagcgccttaagtgcccagcgggctcagaagccccgacgtgtggcggctgagccgggccccgcgcactttctcggccggggaggggttcgggctcgggcacccggagttggcccctcgtaacgccgcgggaaagtgcgggcgagggcagtggactctgaggccggagtcggcggcacccggggcttctagttcggacgcggtgccccctggtggcgctcaccgcgcgcgtggccttggcttccgtgacagcgctcggttggccgtcacagcagccctcggttggccctttcctgctttatagcgtgcaaacctcgccgcgccagggccaagggacaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 5 - DNA, 2954 bp, Homo sapiens
ggcccttccggggctgcgcggctcccccgcctcggtgccggcaaaaatgtgcctagtcacggggccgctctcgggggaactgaggtcgccttcgggctgggacccggagccccttcgccgcgccccaagacctccttgagtgcgggctgcgacgcgctcaccccgctgggccgtctgtgggcgcggctttgcgaagtcatccatctctcggatcactctctggcagccttgagctctcttgaaagcccagccccgggacgagggaggagcgccttaagtgcccagcgggctcagaagccccgacgtgtggcggctgagccgggccccgcgcactttctcggccggggaggggttcgggctcgggcacccggagttggcccctcgtaacgccgcgggaaagtgcgggcgagggcagtggactctgaggccggagtcggcggcacccggggcttctagttcggacgcggtgccccctggtggcgctcaccgcgcgcgtggccttggcttccgtgacagcgctcggttggccgtcacagcagccctcggttggccctttcctgctttatagcgtgcaaacctcgccgcgccagggccaagggacaggttggagctgttgatctgttgcgcaattgctattttccccagagcggctttgtctttggatttagcgtttcagaattgcaattccaaaatgtgacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 6 - DNA, 2769 bp, Homo sapiens
aaatcagggaggcgcagctcctacaccaacgcctttccggggctccgggtgtgtttgttccaactgtttaaactgtttcaaagcgtccgaactccagcgaccttcgcaaacaactctttatctcgcgggcgagagcgctgcccttatttgcgggggagggcaaactgaacgccggcaccggggagctaacggagacctcctctaggtcccccgcctgctgggaccccagctggcagtcccttcccgcccccggaccgcgagcttcttgcgtcagccccggcgcgggtgggggattttcggaagctcagcccgcgcggccggcgggggaaggaagggcccggactcttgccccgcccttgtggggcgggaggcggagcggggcaggggcccgccggcgtgtagccgattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 7 - DNA, 2640 bp, Homo sapiens
aaatcagggaggcgcagctcctacaccaacgcctttccggggctccgggtgtgtttgttccaactgtttaaactgtttcaaagcgtccgaactccagcgaccttcgcaaacaactctttatctcgcgggcgagagcgctgcccttatttgcgggggagggcaaactgaacgccggcaccggggagctaacggagacctcctctaggtcccccgcctgctgggaccccagctggcagtcccttcccgcccccggaccgcgagcttcttgcgtcagccccggcgcgggtgggggattttcggaagctcagcccgcgcggccggcgggggaaggaagggcccggactcttgccccgcccttgtggggcgggaggcggagcggggcaggggcccgccggcgtgtagccgattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 8 - DNA, 2917 bp, Homo sapiens
ggcccttccggggctgcgcggctcccccgcctcggtgccggcaaaaatgtgcctagtcacggggccgctctcgggggaactgaggtcgccttcgggctgggacccggagccccttcgccgcgccccaagacctccttgagtgcgggctgcgacgcgctcaccccgctgggccgtctgtgggcgcggctttgcgaagtcatccatctctcggatcactctctggcagccttgagctctcttgaaagcccagccccgggacgagggaggagcgccttaagtgcccagcgggctcagaagccccgacgtgtggcggctgagccgggccccgcgcactttctcggccggggaggggttcgggctcgggcacccggagttggcccctcgtaacgccgcgggaaagtgcgggcgagggcagtggactctgaggccggagtcggcggcacccggggcttctagttcggacgcggtgccccctggtggcgctcaccgcgcgcgtggccttggcttccgtgacagcgctcggttggccgtcacagcagccctcggttggccctttcctgctttatagcgtgcaaacctcgccgcgccagggccaagggacaggttggagctgttgatctgttgcgcaattgctattttccccagagcggctttgtctttggatttagcgtttcagaattgcaattccaaaatgtgtaagacgggatattctcttctgtgctgtcaagggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 9 - DNA, 21580 bp, Homo sapiens
agagacgggattttgccatgttggccaggctggtcttgaattcctgacctcccgtgattcacccgcctcagcctcccaaagtgctgggattacaggtaggagccaccacacctggcctggcctgtagttttattggctaagtctggtagccctagcatgggtggagaaggctgtagctgtggaagcccagccagtctgcatctgaggcagggaaaggcagctaaggcagcctgccgtcagcccattgacctgacagataggggccaatgtggcttactcctcatctagcctacccttgtgccgtcctctccaacatccatccacctgacagatggatgacccttcctcttccccaggggttgttttgatgagtagatgacatttctaaagagcgcttagtgcactgcctgacacctggtaagtggtcaagatggggagaactgattgtttctactattctttccctctcacagtggccaccgcagcgctaaacacggtcggtcggcattcagtgctcgccgtggaagcgaaccgcaggagcatcctggggcagcggggagttgctgcactcacctagacaggcggcctactgtgacttcaggcaagtcattttccttctctgtgcctcagtttctttgtctgcaaaatgagcagctggactagaaacttaccattctttcacagcacgtcaaactcaacatgcccatttgagctgaagacactgaggctactcactgggtgtgaacctttgacagcatccaggccctgcacagttgtctaggagaagcctgacccatgatgtcctttccgctacccacctgccacgagccccagttctgagctggaggggtgggggtgggtggaaggtggatagtggtcagtgcctcacctggccgactgcagcctggcagagtccatgggggacctcgcagcacagtggatcctcttgccttgtgtctctagttggtccagctctggtctattccatgctattccgttgtcatgggcaggactgggtttgatgaagttggacctaggtgtatcccagcctctcctggtttctgtcgtctttctgtggtgcccagggcttatgtttctatctctttgaatgttgagtttactccgtccttcagggatcagggctgcctgagctcagggtggaggtgctgttgtctgtctggcttccgggccctggactggcagtgtcccccagcacccctgtccagctatgccaagccccctccttttctttcttggcctctgccctatttgttctgtgactggcccaagagaggggtgtgggctggcaggtgtcccacataaagcctggaggcagcatccaggcctggctgacttaccccggggccccctgcctactgcccccagccaagctgggtctccttcttcctgcccagctgaggcagggcctagaagggcaccgaggcaggccctctgtgatgctcctgttgctatggtaattctagatgagaaggccatagcattctgtgtctggcacccccgcgcatctccccagctctgccctgctgccccaagagatggctctctgctggggagctcgagagcttggaatctgctgcagtcaaagtttcttccatgcggccaggcacagtggctcatgcctgcaatcctagcactttgggaggccgaggcgggtgaattacttgaggtcaggagttcaagaccaacctggccaacatggtgaaaccccatctctactaaaaatacaatacttagccggaaatcacttgaacccaggaggcagatgttgcagtgagccaagatcatgccactgcactccagcctgggtaacaaagcgagactccgtctcaaaaaaaaaaaaaaagtatctttcatgcaggccccatcctttggacctcccttcaaccccaagagagaccactaccctagcaggtaagactctacctctcagaaactgacctttactgtagagctgatagtgggaaagggctcaggatccaaaatcaaaagacctagatcctagtctcaagccatttatagctgtgtgaccttggacaaggcaggccttgagcttcagtgtcctcctttgtaaaatgtggataaacatgcgatactgaatcatccttcaggaatagctctatgactttccgtaggttgttcccctgatgtgtaatgctcttccattcttctcagctccatgaactcctactcatgcttcaagacccaaccgaaatgccccattgtctgtaaaaatttccctgacctctgtgcacttggcacagatcttaggccttgccattctgtgttagagttgttatttaagtgtttccaaattttttgactatgaatacgtacaggcaggaatcagattaggctcagttctggaaggagaaatggtttggcgaatattttctttgcggaacggaaagcagaagagggcgaccttgactgggttgggtgccatctctgcaggggtcaatcctgggactcttgttccaggcatggcttgggtcaatcagcactcatgactttgctgtgtgcctgagccgtgcctggctaactggggacattttcatagctgagacccagtaggagagaccatgacttgtgccagggtgtgcctgtctcagctctggggcagggccaggagcagctgctcatgggatgtctctggggacatgctggggcctctgggaggggaagggagactggccatgcactgtctatggtcctggtggtactcctgggatattgccaaggagcctgcggttcagggaacagggaggagggactctagtgtctccaactccctctcatggctctatggctgctggtcggggtcagggagatgaagcagtgccagctccgtggtctgcactgatcctgaccagtgaaccaggcttccccagccagagccagctagtcaagcacgctctctagtggccaccacagggaaggcaagaacttgtctccacaaaggcaggattttcatttattccttcagagagtacagaatgagcatcaatgttccaggcatggtgccaggcatgtgtgttaactttagttaaaacatacttattggccgagtgtggcggctcactcctgtaatcccaccactttgggaggcctaggtgggaggactgcttgaacttagagctcaataccagcctgggtaatgtagtgagaccctatctctataaaaaaaaaaaaaatagctgggtgtggtggtgtgcgtctgtagtcccagcgacttgggaggctgagatgggaggattgcttgagcctgggaagtggaggttgcaatgagccaaaatcacacccattgcactccagcctgggtgaccttgtctcaaaaatatagttatatatttacttatatatttatatgtataaatatatgtgtacatacaaatatatttgtaatattatagacatattaatacatatttacatatgttaatatataaatatatataatatatatttatcatcttctaaatggaaagctcttgagtttcaggatgtgttggttgggaagggaatacaggatagggaaataatccattaagcaatgactcagcacaggccataaaatcagaatcacctggagaacttgaacaatcccaacacctggggtccatccagaccaatcaaatcagaattcgtaggggtcaggcacaggcaccagtggtttggagagttgacggacacatgtcccatccatcattctcattcggctgccaaggtaggcatcgttgtcccattttgcatatgaggagactgaggctgaagtgaacctgcttctttccccatatcagttgaggacagcgatggggcagcggggaggcagtaaatactcctacaaggctgcctctaaagagagtcttataataacaacagctttattgtttgccatgattagaaaagtattcaaggtcattgcgaaatagtgtttttttttctaggcagataattatataaaggcaaaaatggtgtgtgggggtgggggacaaggggagggagagcattaggacaaatacctaatgcacgcaaggcttaaaacctagatgatgggttgacaggtgcagcaaaccaccatggcacacgtatacctatgtaacaaacctgcacatcatgtatacctatgtaacaaacctgtacattctgcacacgtatcccagaacttaaagtgaaaaaaaaagtggtgtgtagaaaaatcacctgcaatctcagcatagttaacgcttagtacatttcagagagagagggtgacaggaaagggaggatgagagtgggtttaagacacaaggtcatattataaaatcagggcttctggaagtttagtcccaaaaccacacatctcataatcccctgcagtgcttgattaaaatgcaacatccctaaggccacagactcagactctggagaaagatccagaaaactgcccgtttaataaacatttgggcgattcttacggcctctaaagaccaagaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggcacagccagccagtgtggctgcagcaaaacacacagggaaagaatggagggggcatcaatcactgcttcagtaaatttttattgagcgccttctacgagaacacaagaggagcttccattctgaggaggaaacaggcaggaaacaggcagatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggagctaacgaacaagatgggctgagaacactgcctagcccagaggacctgagcttagtgtgtagacattgctgctgttactgcctttgtcattgtattatttatttatttatttatttatttttagacagagttttgctcttcttacccaggctggagtgcaatggcgtgatctcagctcactgcaacctccacctcctgggttcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtgtccctctctaccaggacccaagcccacctgctgcagcccactgcctggccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggtaaagctggcagggctgggccggggggcctgggaaggatgtggctggggctgggagctcctgggggcctcccagccagctcaggggccagtgtaccagttcactacaagactaagctgagctcctgaccaggtcctgggcactggagctgaggctggggctgggggctggggagagaaaagaagtcagactgagaggctgctcaagccaggccagcagggttttagccacccttcctccaaccccaggaggacccctggagcccaggctttgtctggccccactcaactggcctgttttactgaatcccacacagactcataggcccacatagtacattaaaaaagagagagagagagagagagagagagagagatggagtctcactgtgttgtccaggctggtctcgaactcctaggctcaagcaatccccctgccttagcctcccaaggggctgggattacaggtgtgagctactgcacttgaccaaccacatggtacttttttttttttttttttttttttgagacagggtttcactccatcacccaggctggagtgcagtgggggcaatcttggctcactgtaacctctgcctcccaggtgcaagcgattctcctgccttagcctcctgagtagctggaattataggcacacaccaccacgcctggctaatttttttttttttctgtatttttagtagagacagggtttcatcatgttggccaggctggtcttgaacccctgacctcaagtgatccacccacctcggcctcccaaagtgctgggattacaggtgtcagccaccatgcacagcccacatggtacattttttaaaattattttttaattaaaatgtttatctaaggccagtagcagtgactcgcgtctgtaatcccagcactttgaggggccaaggtgcggggatcacttgagcctgggagttcagcgtgggcaacatagtgagaccccgtctctaccaaaaatttaaaaaattagctgggagtggtggcatttgcctgtggtcccagctacttgggaagctgaggtgtggggatggctgaagcctgtgaggtcgaggctgcagtgagctatgatcacaccactgcacttcagcctgagtgacaggctatctcaaaagcaaacaaaataatgtttatctaaacaataaaatataatcacagaatatatgatagcattttaaattgaaaaagcattaatgattacatggattgtaaaatatcaaatacatgaaattcttgtgttcttaataatgctagcaacaaggcacatttggtttttactagggcaccaaggtactttaaaaaaagttagggccagccacaggggctcacacctgtaatcccagcactttgggaggccaaggcaggaggatcacttgagcccaggagtttaggacctgagcaacatagggagatcctgatcctgtctctataaaaaattaaaaaattggctaggccctttggcttacacccgtaatcccagcactttgggaggccgaggcgggtggatcatgaggtcaggagttcaagaccagcctggccaacatagtgaacccaatctctactaaaaatacaaaaattagccgagtggggtggcacgcacctgtagttccagctactcaggaggatgaggccggagaatcgcttgagcccgggaggcagaggttgcagtgagccgagaccatgccattgcactccagcctaggtgacagagtgagactccgtctaaaaataaaataaaaatcaaaaaatgatctgggcatggtggcttatgcctgtagtcccacccagctcctcaggaggctgaagcgggaggattgcttgagcccaggaggttgaggctgcagtgagtcatgactgtgccactgcccttgagcctgggtaacagagcaagaccctatctcaaaacaaacaaacaaacaaacaaacaaacaaaaaccaataaaccaaaaacatttatctaaacaataaaataaaggacagatataatcaccgaatatatgatagcattttaaattgaaaaagcactaatgactacaatggattataaaacatcaaatacataaaattcttaagttcctcctaataccaaatacaaagcacattggtctttggtttttacttgggcactaatgcatgctgaaaaagagtcagacttcattttttagagtagttttaggttcacagcaaaattgagcagaaggtagagttctcatgtgtctctttgctcctccccctgcccccagcctccccactatcaacacccccacactacagtggtagatttattacaatccctgaacccacagtgacacatcactatcacccaaagttcatagcgtacagcagggttcactcttgggctgtacattccatgggtttggataaatgtgtaatgatgtctccaccatcacagcatcaggcagagtagtttcactgctctaacaaaatcctctgcctattcacccctctcattaaagccaaacactctgtttctttttttctttttagagacagtgtctcgctctgtcacccaggctgaagtgcaatggcaatcacagcccattgcagcctccaactcctgggctgaagtgatcctcctatctcagcctccagtggctacgactgcaggcatacggcaacggcacccaactaattttttgtagagatagggtcttgctatgttgcccaggctggtcttgaactcttggtcctgccttagcctcccagagctctgggattacaggcgtgaaccaccgtgcccgtcccaaacactctgtttcgacctgcttttaaacaactgacccttggctgcattcaaaggatcagggtgtctgaaactggcctctgcagcaggaccttccttcctacacatctcccagtggccagtgtgaggattctccccacaagaaaccactggagggggcctcctcctgtccgggtttggggctgtacaaggagcatcatggacctggctcaggcctcaggaggggccctgggctggggaaaatgtgggatagcatcgaggcagtcccactcctacccagggccgggctagacctggggacagtctcagccatctcctcgctgcgtccacacaattccacccccacccccacccccaggctggccctcacggaagaacaacagctgatgtttgagaaactgactctgtattgcgacagctacatccagctcatccccatttccttcgtgctgggtgagttcccccttctggctgttccgggtccctgtggccgcccaggctccagacaggccaggggaggatcacgaggagctgcggcaaggggctggggagggggcgggggaacgccagcggcaggtcggcgcctctctgtagggaaaggtgcggactgcagccagtgaaactgaagttagacgttaggtaagacgtcctgccgttagcaatgaaaaccccattttctgagggaagcgctgacatcatggtccctggagcccctgcgcgggaggggagggggtctggcggatttctgggaccagcagggggacccccgggtgacagaacccttggggctctcgcgcctccatgcgaggctctgcctgcctctcgctcccgagcgccttccaggagggctgggggctaggcccgctcgcagcagaaagctggaggagccgaggcatcgccgggcgctgggccctgggctctggccgcagcctggcccctcgcccctcgccccccgcccctcctgcccaggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggtgggcggaccgggagcaacggggaggcaccgggcagagccaggggccgagatgggcgcggcaggaatggaagatgggtggagccaaagtcccccggactcgggggattgggtggagccaggagtggggtgtagtcaagatttgggggtccaattgggcgggacagagtcgggtgtctgaaggtggggcgaggccaggagcccaccctccgagagtaggagtctgaggcagggctaaggacccttgagggataatggaaagaagggtgacggcttgggaactggtgaggtactagggtctacttccctctgcccttgcccctcttgatctccggtttccactctggaggtatgggacattggtctctgacaccccctcagcctggcctgacctggtcctggttaataagacagacccaggctaggcgtggtggctgtcgcctgtaatcccagtgctttaggaggcaaaggtgggaagatcgcttgagcccagctgtttgagacgcccctgagcaacatagcgagacccccatctctacaaaaacattaaaaattagcagggcatggtggcgtgtgcctgcagtctgaggctgagtatcgggaggctgaggcaggaggatcacttgagcccagcagttccaggctgcagtgcgctaagatcgcaccgctgcactccaacctcggtgacagagccagaccctttctctggaaataaataaataccctgcccacatgctcagcccagaacagcacctagtaggtgctcagaaatttttttgttgttgaaagaaagaggatggcaaaggagtgctgaggttcctataggtcagcaggtgccggccatcccttctgcaggttctcccacccaccgccttcttcactccactctgcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgtgagcccactgtacagacagggctgccgcagagtgggaagggctgtggtccacaggaaacaaggtttcctacaaagagaagccttgggcccctgagggtcttccgagagcctgaggtggggttgcagaatcttttccaacagcaatccacagcccgaggtggtcccttctcagaggcccctccctcttctccaagtctgtgaggtcctggttcccttttgatagatgaggaagctgagacacaaagaggtttagtgagcttcccatggccacacagccaggaatggaccataggtaccaggccctggtacctggagaagaggtgggggcgagcccagggtgggggcaggtggtgttcagaaccccatccccctcttctgccccccaggagatgaacaccttgcgtactcagtgtggacacctgtatgcctacgactggattagtatcccactggtgtatacacaggtgaggactaggctggtgaggctgcccttttgggaaactgaggctagaaggaccaaggaagcagctggggtgggaagggctcacctagaggctaagtggctcccctgggagttgggtccacactttgaagttgggtctggactttgaagtgccaagttctaagagtccaggctcctgcctggcccagtccagtagaggcaatgtgattatccccatattaaagagaggttggccgggtgcagtggctcatgcctgtaatcccagcactttgggaagctgaggcaggtggatcacctgaggtcaggagttcgagaccagcctggccaacatggtgaaaccccatctctactgaaaatacagaattagctgtgtggtggtgcacgcctgtaatcccagctacttgggaggctgaggcaggagaatcgcttgaacccgggaggtggaggttgcagtgagctgagatcatgccactgcactccagcctgggcgacacagcaagactctgtctcaaacaaacaaacaaacaaacaaacaaaggggttaacagagcccctaagtcacataagtgtgcaagtcagaacaaggccttggtctcctgtctcagactcccagcccctggagcatcctgatttcagggttcccacctagccctttgctaccacatcctcctcctcctcccaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtgggcctctccagggccctgctgggctggaggcatggccagaggggtcatggccagcagctgcctgagacgaggatgcagtgtcaggaaaggaaggtctcacgggtagaaagcagccaggcgtggtggcgcacacctgtaatcccagctactcgggaggctgaggcaggagaatcgcttgaacccgggaggcggaggttgtggtgagttgagatcgtgccactgcactccagcctgggcaaaagaatgaaactctatctcaaaaacaacaacaacaacaaaacaaagccctaaggttcagaagcccctgccctttagaaggcagatcggcaccacctctccttattcaagatgcctgttgggctgtcttgttcctcacctcaagtggcttgtccaggtattccctcccaccacagccaatactccgaacagatgttctgaatcacacagttttctctccacctctttatctttccttccttctgttgcccacccactctctctcccttcctaccttcctttattttttggtaatgggggtgtaagtctctgtctctgcccttcctgtcactgtgacacacacacacacacacacacacacacacacatacacacacacacacacgcattcctattcctctaaattccccctgcccccccagttatctttggtttctgcagatcaaaacaaatcacacttttatgcttgaaattctccggggtgccccagtggcctgcaagatgtcccctggacccctaaggcagacgcgtgtcacctcttcggggctttgttagggcattttagaggttgctatccaggaatctgcccacctagactgccctttagttcagcccagcttcagtatatatctctgttgcatgaatgaataaaattatgcaactccaggtaagatacatgaggtgagataaaggcagtgactcagccgagtgatacactcagggacagctgtgggtgttcagggaaggactggctcagaagagttagaggggctgtgtccagaagtgtgtgggtgcccacaagtgtggggggctggagccctaaactctgcctttgaagacagtggtcaggcaggaagggcgtcatggggtgtggaaatagcagcagctgaggtttaaagggggaagctggctttgaggagttctgcctgagggtttacagagcctcacctgtccccaaggtggcagagcagctcatcaacccctttggagaggatgatgatgattttgagaccaactggattgtcgacaggaatttgcaggtatggggagagggagagaaaccataccatggaccttccccaaagtggacccaaagagaggaccccactgttctgtagggaggcctcacagtgaatgatcaacccttcccctcctccctcctgcagccagtcattcactcacaggattctcacctcaatctttgaggctgcaggcaggcacccatctccccatttcacaggcagggaaactgaggtccagagagagggagagattcctccaagtcatcaggcacatacaaggtcctgcctgggatgatctttctgtgggacttcttctgtccctggtgaccaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcaggtgtggccagagccagggggctgggtgggaagcccctcctagtgcaggggtctgcctaggaacttagaatagcactagttaatgcatacaggttgcttcagtaagtgtcaggcactgtactatgctctttataaacattaactatttttttcctcccaataattctggtttgttatcccaagttttcagataattaaagtacaggttcagagagagtaagttgtccaaggccacatagctaccaaatggtgcatttgctactcgaaggacagcctgtgatcagtgatgcagtggaacgttaggacctggctcttgtcatccagaactatgttttcttttctttttgagacagtatctcgctctgtcgcccaggttggagcgcagtggcgtgatcttggctcactgcaacctccgcctcctgggttcaagtgattctcctgcttcagcctccccagtagctgggattacaggtgcccacaaccacaactggctaatttttgtacttttagtagagatgaggtttcaccatgttggccaggctggtctccaactcctgaccagtaatctgcccgctttggcctcccaaaatgctggaattataggtgtcaaaactatgttttctgataagctacgatgcttggatgggaagtggaagtggggttccctgggatgggggaggggcagcaaagtcccagcaggcagccaggccatcacaggtacctcctgaattgactttgtcctaccgagtaaagggctcaggccacccacagcagccagacttatccccacatggtcccacttccctgattccatctgaatccctcttgagctgcagtgggctgaagggctatcccagctggtcctttctccccaggacaacagagttgaaagtgccttggagagtgttgggcacatgtcagggttcatactcaagggtttcttccacggtatccagtgctgttctcgcttgttcttttctttttttttttttaaacggagtttcactcttgttgcccagagctggagtgcagtggcataatctcggctcactgcaacctccgcctcccagattcaagcaattctcctgcctcagcctcctgagtagctgggattataggtgccagccaccaagcccggctaatttttgtatttttagtagagacagtttcaccatgttggccaggctggtctcgaactcctgacctcaggtgatccaccctcctcagcctcccaaagtgctgggattacatgtgtgagccactgtgcctggctgcttgttcttttaagaaccaaatatcctactagactgcaatcgagtttaactacagtctatagatagtgtgaggaagggttgggaaggtgatcaaatgaaggctggaggcttgcttaggtcagaaacatttctggaggatgactttgagccctacatggtctgtaccccagcagctgaaggttgttgaggggtggggagggctgaaaacagaacgataaagcatagaccttgtctccaaggaatgcacaatttatggagggagctcaaacccaagcctcaaactctggatacaaggtacaaagtactggatgtccagaaaagggacagaacatggaacacagtcatctttgtctgcctgggaggcggcttccagctgggtctggagctgagccatggaacatgggaagaatctgaacttgggcaagggcaggccatactctctggtagataagctttcccttgcagggtaaaggtctggggctcccgggatgcctgttgctaggaagtcaaatttctctttgtggatgtcactcccagttggaaccacaaattcctggcattgcccagagtcactcatgggcctcatctgaaccactcatgccagggcaccagtgtttctgactgcctggagtgaggggttttacaggggaagtgaatgatgaggaggcctttacacgccaggcggggtggttgcgggggttggatgttaactctggtcaagagggaatcaacaaacagtgaggtgagctgggcctggagggatcaccgggaggtacaggacagatcaggagagaggtgagagctggggcatggtgaggaagacggtgtggccttggcttgggccaactgagagagaggagcgggggtaagggagaagtaaggccaggtgttggtcctttgtccactggctcagccctgcatctcctgtttctttccagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacaggtctgtcctccacctgaaccaggggcactgcattgccctgtgccccaccccagcttcccttgctctgagcctacccttcctccacaatttcctagggttccatcactgccagagcacactggacctacgcccagcactggcttggggtatatacttggccaccttcacagggatcctagggaagtgttcgggaccttttctcacttcaccctggtatcacccggaagacttcttgggaccaggtgaaggaagatgaggttgtgctgaccagaatgctgctggagaactgccccagggctgacaggccaggcttagctgagcagatgttatcactggccccaacttactttgagcaagggtggctgacccaaaaccatgaggtggcagtcagctggatgacagatgaacacttcccccataactatttagggtagtacccaagcactacaggaaagggtggcaggaactgcctcactcctaggaactggtagatggtgaggttgagggtgtccagcgcccttaggtcattttctcactgcctgggaacctcaccaaaatacttcttgcttccttggggtcagcccaaagctgtcacaaaatcagatatttccctttattccagatttcctggacactttcacccaattataaacaccccacttcagccccaatcacgtgggaggaagtgtaacttcccttttctggattctcaagcagttactttcacggtcagaacacgcagctattatgattgaaaacttaaaagggcaacaatttcagtcttgcttctagggctagacaggaacttggcaaacatctgtggcctgttcagcaaaggatgttaatatttaagaatcttgtcttgggctgggtgtggaggcaagtggatcacaggaggtcaggagtttgagaccaacctggccaacatgatgaaaccccatctctaccaaaaaaaatacaaatcagctgggcgtcgtggtgtgcctgtagtcccaacgcaggaggttgaggggagaattgcttgaacccaggaggtggtggttgcagtgagattgagcaactgcaatccagcctgggcgacggagtgagactgtctcaaaaaaaaaaaaaaaaggatcgtctcaacctttgccctcctactgcaacattttggtatttgaaatgaagggaccttccatacttatgctgttaatactttcattctcactagggatgaagcacattcctaacctgcttcctaatggggatgcttcgccagccaggtcctcacctgtgtgtacaccagcaggacactgatccagtcacagccatacagctgtccacactgaagaacatgtcctacaacagcctgaatcaaatggttagcttaatagataaaaatcccagactacttcagcctttaatgccttttattcataaaaactgtgaaagctagactgaaccattggaaacatttaactcagactctggattcagagtcgggaacccttagttctatctgaatccaagacagccacaccttagtatactgcccaaactaatgagtttaataaatacaaatactcgtttctttttgattagtgtgattagaactgaacaacggcacttaaggaatctggaagatagcctggatagatttctgattcatcccaagacctcaaagacaacacctgggtaccaaatttctttatttgaaggaatggtacaaatcaaagaacttaagtggatgttttggtacaacttatagaaaaggtaaaggaaaccccaacatgcatgcactgccttggtgaccagggaagtcaccccacggctatggggaaattagcccgaggcttagctttcattatcactgtctcccagggtgtgcttgtcaaagagatattccgccaagccagattcgggcgctcccatcttgcgcaagttggtcacgtggtcacccaattctttgatggctttcacctgctcattcaggtaatgtgtctcaatgaagtcacacaactgcaaaacaatggggaagacagttagtgggcagctttcccaatccctaaggcaaatgatttcctccatttatttcctggggttccaatactcacatgggggtcatttttgtcagtggccagtttgtgcagttccagtagtgactgattcacatttttttccaaatgtaatgcacactccattgcattcagcccgctctcccagtcatcacagtctggtttctgaatgagaataggttaatgcatctctaccaactaacctagaagtcagcaagcccatcatctctaaccaccacgttttggcaaaagggacattactatttgcctaatttagtttagatggtaagcctaaaaaagcacaaaaggcaaaagcccagtgtatcatcactttataagggcaattgctagtttaagtgatttctgatacccgaacactgccagatgatgaagtatgacacccctaggatcttttgttcaccttgatatcctgaaggaagattcggccacctcgttggttctgcagcttcatcagtttctcagcatgttccctctcctcatgagattggtgaagaaagtatttggcaaagttcttcaaagccacatcatcgcggtcaaagtagtaagactgaaaggggaacactgaatgtgttatactagggatccccagagacaggtagctgtgacgatctacagggaggcaagatggtctcgtcagcctaggcttcctttctcaaagcacagcatgaaggaatcagtgcctaaggagactggggggcagatgagctactggattgaacccaagagtaatgtttctattctaaaatagaaaactgaaacttgatccttgaaacttaacttcaaggaacctttaggatattatgggcattgcctggggcaatgtcttaaggatctgtattactgcacagaaaacttacagccagtgtcagaattcattgtctgatgagagggcgctggagtactgaccctgcacttagtccttttctcctccctgctcctgtattaaccatgttaaagcatggttaaaactgagctcaggagtctgccagctgtaccacatatttatttataaaattaaaaacccctgctgggtgcggtggctcagcctgtaatcccagcactttgggaggccgaggcaggcggatcacgaggtcagcagatcgagaccatcctggtcaacatggtgaaaccccgtctctactaaaaataaaaaaaaatagctgggcgtggtggcgtgcgcctgtaattccagctactcaggagcctgaggtaagagaatcacttgaacccaggaggcggaggctgcagtgagccgagatcgcgccactgcactccagcctgggcgacagagcgagactctgtctctcacacacacacacaaattaaaaacccccatgcaaggtggcaaatgtggacagtatgttgatgtaaacatacttttataatagttatatcacaaaaatttgattttcagttttagacatttgatgtagtcaagtgtttgtgaaaggcagtgacagtcacagaaatacaggcctgttttcaaatccatttg

SEQ ID NO: 10 - DNA, 1818 bp, Homo sapiens
atgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactag

SEQ ID NO: 11 - DNA, 1770 bp, Homo sapiens
atggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactag

SEQ ID NO: 12 - DNA, 1749 bp, Homo sapiens
atggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactag

SEQ ID NO: 13 - DNA, 1770 bp, Homo sapiens; identical to SEQ ID NO: 11

SEQ ID NO: 14 - DNA, 1770 bp, Homo sapiens; identical to SEQ ID NO: 11

SEQ ID NO: 15 - DNA, 1728 bp, Homo sapiens
atgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactag

SEQ ID NO: 16 - DNA, 1599 bp, Homo sapiens
atgatggacttggagctgccgccgccgggactcccgtcccagcaggacatggatttgattgacatactttggaggcaagatatagatcttggagttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactag

SEQ ID NO: 17 - DNA, 1518 bp, Homo sapiens
atgaagagacaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactag

SEQ ID NO: 18 - DNA, 592 bp, Artificial Sequence
  Description of Artificial Sequence Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 19 - DNA, 141 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 20 - DNA, 141 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 6653 bp, Artificial Sequence
  Description of Artificial Sequence Synthetic polynucleotide
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacccagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcaattcggtaccatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagaccggtggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 22 - protein, 605 aa, Artificial Sequence
  Description of Artificial Sequence Synthetic polypeptide
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 23 - protein, 131 aa, Artificial Sequence
  Description of Artificial Sequence Synthetic polypeptide
IEGRRDVAEGRPAQNPGGNTGEQPWKGRQLPRQHHGIVSAQQPSPCPAAGKAGGDEFRRGNREGESESPGKELTGGGNAPTSGGCVSKHSAHHATLPDNGPQLLIKRQQPGFIQGGENESHTGSNSMIQRH

SEQ ID NO: 24 - protein, 286 aa, Artificial Sequence
  Description of Artificial Sequence Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVVMVTTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 25 - DNA, 20 bp, Artificial Sequence
  Description of Artificial Sequence Synthetic primer
actctttccgtcgctgactg

SEQ ID NO: 26 - DNA, 24 bp, Artificial Sequence
  Description of Artificial Sequence Synthetic primer
tgcagcaaacaagagatggcaatg